(19) United States
(12) Patent Application Publication
(10) Pub. No.: US 2003/0166866 A1
(43) Pub. Date: Sep. 4, 2003

(54) Method of processing a proteinaceous material to recover K-casein macropeptide and polymers of a-lactalbumin and B-lactoglobulin

(75) Inventor: Ernest P. Brody, Minneapolis, MN (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Land O' Lakes, Inc., Arden Hills, MN (US)

(21) Appl. No.: 10/058,907

(22) Filed: Jan. 28, 2002

(51) Int. Cl. C07K 14/47

(52) U.S. Cl. 530/361

(57) ABSTRACT

A method of processing a proteinaceous material that includes κ-casein macropeptide, the method entailing polymerizing protein present in the proteinaceous material to yield a proteinaceous intermediate, where the proteinaceous intermediate includes polymerized protein, and separating the proteinaceous intermediate to yield a first portion and a second portion, where the first portion includes a majority of the κ-casein macropeptide from the proteinaceous material and the second portion includes a majority of the polymerized protein from the proteinaceous intermediate.

### CROSS-REFERENCE TO RELATED APPLICATION(S)

None

### BACKGROUND OF THE INVENTION

The present invention generally relates to a method of processing a proteinaceous material, such as a dairy material, where the proteinaceous material includes at least κ-casein macropeptide, α-lactalbumin, or β-lactoglobulin, and more preferably includes κ-casein macropeptide and at least α-lactalbumin or β-lactoglobulin. More specifically, the present invention relates to a method of recovering κ-casein macropeptide from the proteinaceous material and to a method of recovering derivative(s) of α-lactalbumin originally present in the proteinaceous material and/or derivatives of β-lactoglobulin originally present in the proteinaceous material, while at least substantially maintaining the solubility of β-lactalbumin derivative(s) and/or β-lactoglobulin derivative(s) that are recovered.

More than 100 million pounds of fluid whey is produced worldwide annually. Fluid whey is an opaque, greenish-yellow fluid that typically contains about 5 to about 7 weight percent total solids, with the balance of the fluid whey being water. The solids of fluid whey primarily include water-soluble proteins, water-insoluble proteins, fats, carbohydrates, and ash.

Fluid whey has a very high biological oxygen demand (BOD). Because of the high BOD, disposal of fluid whey by application to land or in water courses, such as creeks and rivers, is typically illegal in most developed countries. Furthermore, treatment of fluid whey in waste water treatment plants to reduce the BOD level of the fluid whey is relatively expensive. The inherent difficulties that fluid whey disposal create have spurred development of processing technologies that render fluid whey, or components of fluid whey, useful in preparing food products for human and animal consumption.

Cheese manufacture is the source of most fluid whey. Cheese is made from the milk of various mammals, such as cattle, sheep, goats, reindeer, and buffalo. Cheeses produced from the milk of different animals often have differences in texture and taste, largely due to the composition of milk being different between different types of animals. There are two major categories of proteins contained in milk. The first type of milk protein exists as a suspension (colloid) in milk and is known as casein, while the second type of milk protein is soluble in the milk and is commonly referred to as whey protein. Beyond these two major protein categories, other proteinaceous components of milk include peptones, non-proteinaceous nitrogenous compounds, and various enzymes.

Cheese manufacture is initiated by separation of the casein protein components of milk from the whey protein components of milk. In the cheese industry, two types of precipitation techniques are most commonly used to separate the overall milk protein fraction into caseins and whey proteins. These two techniques are rennet precipitation and acid precipitation. The by-product fraction produced during cheese manufacture that includes the whey proteins is commonly referred to as fluid whey. Fluid whey is further defined with reference to the type of coagulation that is employed to separate the casein fraction and the whey protein fractions.

Fluid wheys that result from rennet precipitation are commonly referred to as sweet wheys, whereas fluid wheys that result from acid precipitation of caseins are commonly referred to as acid wheys. Besides sweet whey and acid whey, the cheese industry also produces mixtures of sweet whey(s) and acid whey(s). When this condition exists, the whey that results is named, either as sweet whey or acid whey, in terms of the particular coagulation process (rennet precipitation or acid precipitation) that is considered to prevail over the other coagulation(s) employed in the particular cheese manufacturing process.

The rennet precipitation process that produces sweet whey is characterized by the addition of the rennet enzyme (chymosin) to milk to cause precipitation of the caseins that are present in the milk. In rennet enzyme precipitation, the rennet enzyme is added to warm milk (30° C.-35° C.). The added rennet enzyme causes caseins to precipitate from the milk and leaves water-soluble whey proteins in solution following removal of precipitated caseins. The rennet enzyme acts on a particular casein component, κ-casein, of the milk to cleave the peptide bond between two particular amino acids, namely phenylalanine #105 and methionine #106, within the κ-casein. Thereby, the rennet enzyme effectively fractionates the κ-casein into para-casein and κ-casein macropeptide. The para-casein precipitates along with other caseins (α-, β-, γ-caseins) of the milk and the precipitated caseins are further processed to form cheese.

κ-casein macropeptide is commonly referred to as caseinomacropeptide, casein macropeptide, or simply as CMP. κ-casein macropeptide may be glycosylated and therefore may be present as glycosylated CMP and/or as non-glycosylated CMP. Additionally, both glycosylated CMP and non-glycosylated CMP may be phosphorylated and therefore may be present as (1) phosphorylated, glycosylated CMP; (2) non-phosphorylated, glycosylated CMP; (3) phosphorylated, non-glycosylated CMP; and/or (4) non-phosphorylated, non-glycosylated CMP. As used herein, the term “κ-casein macropeptide” (as well as the terms “casein macropeptide,” “caseinomacropeptide,” and “CMP”) refer collectively to all forms of κ-casein macropeptide ranging from (1) phosphorylated, glycosylated CMP; to (2) non-phosphorylated, glycosylated CMP; to (3) phosphorylated, non-glycosylated CMP; and to (4) non-phosphorylated, non-glycosylated CMP. On the other hand, as used herein, the term “κ-casein glycomacropeptide” (as well as the terms “casein glycomacropeptide,” “glycomacropeptide,” and “GMP”) each refer collectively to (1) phosphorylated, glycosylated CMP and to (2) non-phosphorylated, glycosylated CMP.

As opposed to processes that employ rennet enzyme and produce sweet cheese whey, acid whey results from the addition of acid, at the iso-electric point of casein (i.e. at pH˜4.7), to precipitate casein and leave water-soluble whey proteins in solution. The acid precipitation technique does not typically cause fractionation of κ-casein, but instead causes precipitation of all of the caseins in the milk. Consequently, processes that rely entirely upon acid addition to the milk, without any rennet addition, typically leaves a solution of water-soluble whey proteins that contains no, or essentially no, κ-casein macropeptide (CMP), and consequently no, or essentially no, glycomacropeptide (GMP).

Nonetheless, as indicated above, some cheese manufacturing processes do exist that primarily rely on acid precipitation while also including a minor amount of rennet enzyme precipitation. These types of cheese manufacturing processes do produce fluid whey that includes a significant amount of κ-casein macropeptide (CMP), and consequently a significant amount of glycomacropeptide (GMP), though these types of combination processes do not produce fluid whey with κ-casein macropeptide (CMP) and glycomacropeptide (GMP) contents that are as large as those resulting from pure sweet whey (rennet enzyme addition) production processes.

The choice between the methods (rennet precipitation or acid precipitation) used to precipitate casein depends upon the particular cheese product that is desired. Some examples of cheeses that are made by processes are at least in part based upon rennet enzyme addition and have sweet whey as a byproduct include Gruyere, Cantal, Swiss, provolone and cheddar. One notable example of cheese that has acid whey as a byproduct is cottage cheese.

Besides any κ-casein macropeptide (CMP) and any consequent glycomacropeptide (GMP), whey produced during cheese manufacture also includes various other water-soluble proteins such as β-lactoglobulin and α-lactalbumin; some water-insoluble proteins; carbohydrates that are primarily in the form of milk sugars, such as lactose; water-soluble minerals and vitamins; various enzymes; ash; and water. The various protein compounds that may be present in fluid whey have received wide attention for their potential utilization in various foods, feeds, and other products.

For example, glycomacropeptide (GMP) has a number of potential therapeutic uses and functional properties that make it very useful as an ingredient in food and feed compositions. GMP may be used as a nutritional component in diets of persons suffering from phenylketonuria. Phenylketonurics lack phenylalanine hydroxylase in their metabolic system. Therefore, phenylketonurics are unable to utilize phenylalanine present in foods. Consequently, if phenylketonurics consume food that contains phenylalanine, the phenylalanine may accumulate in the phenylketonurics and cause irreversible mental retardation. An important feature of GMP is that GMP does not contain any aromatic amino acids, such as phenylalanine. Consequently, use of GMP in the diets of phenylketonurics may help prevent accumulation of phenylalanine in people who suffer from phenylketonuria.

Besides this important therapeutic use for phenylketonurics, GMP also possesses a wide range of biological activities (bioactivity). The particular bioactivity exhibited by GMP is believed to depend upon a number of different variables. For example, the type of glycosylation, the position(s) of glycosylation along the peptide chain, and the character of the peptide chain itself may dictate the particular bioactivity of a particular GMP form. Thus, a variety of glycosylation variables, such as glycosylation factors and peptide chain factors may combine to affect the particular bioactivity that is exhibited by a particular GMP form.

Against this background of variables that may affect GMP bioactivity, some forms of GMP bind to cholera enterotoxins and to E. coli enterotoxins and thereby help render these substances inactive. Also, some forms of GMP bind to bacteria and thereby help inhibit binding of the bacteria to some surfaces and consequent colonization of the bacteria. For example, some forms of GMP bind to cariogenic bacteria and help prevent the cariogenic bacteria from binding to tooth surfaces. Also, some forms of GMP inhibit binding of some bacteria and some viruses to the intestine or other mucosal surfaces and consequently help block and inhibit colonization of these particular bacteria and viruses.

Additionally, investigation of some forms of GMP is underway to determine of these GMP forms may help inhibit or depress digestion of milk-derived immunoproteins in the stomach and thereby allow these proteins to pass intact through the stomach and provide a protective effect in the intestine. If such a protective effect by GMP is identified, the subject GMP forms may help improve feeding of infants and may help improve the health of immunocompromised patients. Various other potential uses of particular GMP forms in the diets of humans and animals, such as the potential for particular GMP forms to favorably modulate immune system responses, have been identified or are under investigation.

Beyond the variety of different variables that may affect GMP bioactivity, there are a number of related variables that may affect the bioactivity of other κ-casein macropeptides. The diversity of molecular species present in mixtures of different κ-casein macropeptides helps enhance the range of protections and bioactivities exhibited by the mixture. Some of the biological activity is related to the primary and secondary structure of the peptide chains in the different κ-casein macropeptides as well as the nature of any phosphorylation on the peptides of the different κ-casein macropeptides. Other portions of the bioactivity are determined by the nature of any glycosylations on the peptides of the different κ-casein macropeptides, such as glycosylations that include a non-sialic acid containing monosaccharide, glycosylations that include a non-sialic acid containing disaccharide, glycosylations that include two positionally-different sialic acid containing trisaccharides, and glycosylations that include a disialic acid containing tetrasaccharide. In addition, there are several possible threonines where the saccharides may attach during glycosylation of different non-glycosylated κ-casein macropeptides. Further, multiple glycosylations in different κ-casein macropeptides may exist.

There are indications that both the amino acid chain structure of the peptides in different κ-casein macropeptides and the identity (character) and positioning of the glycosylation(s) in different κ-casein macropeptides help determine the protection(s) and bioactivity (bioactivities) exhibited by different κ-casein macropeptides and consequently by mixtures of different κ-casein macropeptides. Thus, a variety of glycosylation variables, such as glycosylation factors and peptide chain factors, may combine to affect the particular bioactivity that is exhibited by particular κ-casein macropeptides. Consequently, it is beneficial to obtain mixtures of different κ-casein macropeptides that maximize representation of different variables, such as glycosylation variables, to enhance the overall variety of protections and biological activities exhibited by the mixtures.

While κ-casein macropeptides, such as GMP, are an important water-soluble protein component of some fluid wheys, α-lactalbumin and β-lactoglobulin are the predominant proteins in fluid whey. Also, α-lactalbumin and β-lactoglobulin are generally present both in acid wheys and sweet wheys. Both α-lactalbumin and β-lactoglobulin are proteins that are frequently the subject of recovery from fluid whey because of beneficial functional properties and utilities of these two particular proteins.

Processes exist for producing a whey protein concentrate (WPC) that has a higher concentration of α-lactalbumin and β-lactoglobulin as compared to the concentrations of these two proteins in the fluid whey used to produce the whey protein concentrate. However, whey protein concentrates typically have some undesirable properties, such as a tendency for a rather low foamability and poor foam stability, due to the tendency for whey protein concentrates to have a relatively high content of both lactose and lipids. As an alternative to whey protein concentrates, whey protein isolates (WPI) have been produced by absorption of water-soluble proteins, such as κ-lactalbumin and β-lactoglobulin, directly from fluid whey onto ion exchange beads, followed by washing and elution of the absorbed proteins. However, the rate of whey protein isolate production using ion exchange beads is relatively slow and tends to be rather expensive, based upon the cost per pound of recovering high value whey proteins, such as α-lactalbumin and β-lactoglobulin.

Water-soluble proteins, such as κ-casein macropeptide, β-lactoglobulin, and α-lactalbumin, that maybe present in fluid whey have a relatively high value and a number of beneficial uses as products or additives in the food and feed industries, depending upon the particular protein or group of proteins. While protein separation technologies developed to date have increased the overall knowledge base with respect to particular whey proteins, these protein separation techniques remain relatively expensive, based upon the cost per pound to recover particular proteins. Furthermore, many of these processes require a high amount of operator input when attempting to maximize protein recovery while minimizing the time required to recover particular proteins. A need therefore exists for a process that is capable of efficient and cost effective separation of valuable whey proteins that allows full realization of the benefits of whey proteins, such as κ-casein macropeptide, β-lactoglobulin, α-lactalbumin, and derivatives of α-lactalbumin and β-lactoglobulin. The method of the present invention satisfies this need for a cost effective and efficient whey protein separation technique.

### BRIEF SUMMARY OF THE INVENTION

The present invention includes a method of processing a proteinaceous material that includes κ-casein macropeptide. The method entails polymerizing protein present in the proteinaceous material to yield a proteinaceous intermediate, where the proteinaceous intermediate includes polymerized protein. The method also entails separating the proteinaceous intermediate to yield a first portion and a second portion, where the first portion includes a majority of the κ-casein macropeptide from the proteinaceous material and the second portion includes a majority of the polymerized protein from the proteinaceous intermediate. The present invention further includes a polymerized protein material, a κ-casein macropeptide-enriched powder and a method of fractionating a first proteinaceous material or a second proteinaceous material.

### BRIEF DESCRIPTION OF THE DRAWINGS

FIG. 1 is a block flow diagram of a process of the present invention for processing and separating whey proteins.

FIG. 2 is a block flow diagram depicting batch-wise filtration in accordance with the present invention.

FIG. 3 is a block flow diagram depicting continuous filtration in accordance with the present invention.

FIG. 4 is a high performance liquid chromatography (HPLC) plot at a detection wavelength of 280 nanometers that demonstrates an effect of heat treatment on alkaline proteinaceous streams in accordance with the present invention.

FIG. 5 is an HPLC plot at a detection wavelength of 214 nanometers that demonstrates an effect of heat treatment on alkaline proteinaceous streams in accordance with the present invention.

FIG. 6 is an HPLC plot at a detection wavelength of 280 nanometers for a microfiltration permeate and a microfiltration retentate that are derived from the alkaline heat-treated proteinaceous stream depicted in FIG. 4.

FIG. 7 is an HPLC plot at a detection wavelength of 214 nanometers for a microfiltration permeate and a microfiltration retentate that are derived from the alkaline heat-treated proteinaceous stream depicted in FIG. 5.

FIG. 8 is an HPLC plot at a detection wavelength of 280 nanometers for an ultrafiltration retentate and for an ultrafiltration permeate that are derived from the microfiltration permeate of FIG. 6.

FIG. 9 is an HPLC plot at a detection wavelength of 214 nanometers for an ultrafiltration retentate and for an ultrafiltration permeate that are derived from the microfiltration permeate of FIG. 7.

FIG. 10 is an HPLC plot at a detection wavelength of 280 nanometers of another proteinaceous stream and a heated form of the proteinaceous stream that demonstrates heat treatment effects, in accordance with the present invention, on the proteinaceous stream.

FIG. 11 is an HPLC plot at a detection wavelength of 280 nanometers for a microfiltration permeate and a microfiltration retentate that are derived from the heat-treated proteinaceous stream depicted in FIG. 10.

FIG. 12 is an HPLC plot at a detection wavelength of 280 nanometers for an ultrafiltration retentate derived from the microfiltration permeate of FIG. 11.

FIG. 13 is an HPLC plot at a detection wavelength of 280 nanometers for a spray dried powder derived from the ultrafiltration retentate of FIG. 12.

FIG. 14 is an HPLC plot at a detection wavelength of 214 nanometers for the spray dried powder of FIG. 13 that was derived from the ultrafiltration retentate of FIG. 12.

FIG. 15 is a reversed phase HPLC plot at a detection wavelength of 214 nanometers for an aqueous solution of the spray dried powder of FIG. 13 that was derived from the ultrafiltration retentate of FIG. 12 and for a whey protein concentrate precursor of the proteinaceous stream of FIG. 10.

FIG. 16 is a reversed phase HPLC plot at a detection wavelength of 214 nanometers for the aqueous solution of the spray dried powder of FIG. 13 that was derived from the ultrafiltration retentate of FIG. 12 and for a solution of the Sigma volumetric glycomacropeptide standard employed in Protein Analysis Procedure No. 2.

FIG. 17 is an HPLC plot at a detection wavelength of 280 nanometers of another proteinaceous stream and a heated form of the proteinaceous stream that demonstrates heat treatment effects, in accordance with the present invention, on the proteinaceous stream.

FIG. 18 is an HPLC plot at a detection wavelength of 280 nanometers for a microfiltration permeate and a microfiltration retentate that are derived from the heat-treated proteinaceous stream depicted in FIG. 17.

FIG. 19 is an HPLC plot at a detection wavelength of 280 nanometers for an ultrafiltration retentate derived from the microfiltration permeate of FIG. 18.

FIG. 20 is an HPLC plot at a detection wavelength of 280 nanometers for a spray dried powder derived from the ultrafiltration retentate of FIG. 19.

FIG. 21 is an HPLC plot at a detection wavelength of 214 nanometers for the spray dried powder of FIG. 20 that was derived from the ultrafiltration retentate of FIG. 19.

FIG. 22 is a reversed phase HPLC plot at a detection wavelength of 214 nanometers for an aqueous solution of the spray dried powder of FIG. 20 that was derived from the ultrafiltration retentate of FIG. 18 and for a whey protein concentrate similar to a whey protein concentrate precursor of the proteinaceous stream of FIG. 17.

FIG. 23 is a reversed phase HPLC plot at a detection wavelength of 214 nanometers for the aqueous solution of the spray dried powder of FIG. 20 that was derived from the ultrafiltration retentate of FIG. 18 and for a solution of the Sigma volumetric glycomacropeptide standard employed in Protein Analysis Procedure No. 2.

### DETAILED DESCRIPTION

The present invention generally relates to a method of processing a proteinaceous material, such as a dairy material, where the proteinaceous material includes at least κ-casein macropeptide, α-lactalbumin, or β-lactoglobulin, and more preferably includes κ-casein macropeptide and at least either α-lactalbumin or β-lactoglobulin. More specifically, the present invention relates to a method of recovering κ-casein macropeptide from the proteinaceous material and to a method of recovering derivative(s) of α-lactalbumin originally present in the proteinaceous material and/or derivatives of β-lactoglobulin originally present in the proteinaceous material, while at least substantially maintaining the solubility of α-lactalbumin derivative(s) and/or β-lactoglobulin derivative(s) that are recovered.

The method of the present invention generally entails treatment of a fluid proteinaceous material that contains κ-casein macropeptide and one or more heat-labile proteins, such as α-lactalbumin and/or β-lactoglobulin. Preferably, the fluid proteinaceous material includes κ-casein macropeptide and at least either α-lactalbumin or β-lactoglobulin. Still more preferably, the fluid proteinaceous material includes κ-casein macropeptide, α-lactalbumin, and β-lactoglobulin. Nonetheless, it is permissible for the fluid proteinaceous material that is treated in accordance with the present invention to include α-lactalbumin and/or β-lactoglobulin, without any κ-casein macropeptide, though benefits of the present invention are more fully realized when (1) κ-casein macropeptide and (2) α-lactalbumin and/or β-lactoglobulin are present, with both α-lactalbumin and β-lactoglobulin preferably being present along with κ-casein macropeptide.

Briefly, according to the method of the present invention, the fluid proteinaceous material, at an alkaline pH greater than about 7, is heated to a temperature of greater than about 175° F. and is thereafter cooled following a holding period, to a temperature of less than about 140° F. The heating of the alkaline fluid proteinaceous material causes polymerization of any α-lactalbumin and any β-lactoglobulin that is present in the material to yield polymers of α-lactalbumin and β-lactoglobulin, which consists of an aggregated protein, namely, polymerized protein. Surprisingly, it has been found that the polymerized protein retains a substantial amount of the solubility in water and some functionality that was originally possessed by the protein, prior to polymerization, despite the higher molecular weight of the polymerized protein, versus the lower molecular weight originally possessed by the protein, prior to polymerization.

As used herein, the term “heat-labile protein” means a protein that degrades when exposed to heat. A-lactalbumin and β-lactoglobulin are two preferred examples of heat-labile proteins. Degradation of α-lactalbumin and β-lactoglobulin creates residuals of the α-lactalbumin and β-lactoglobulin that polymerize to form polymers of α-lactalbumin and β-lactoglobulin in accordance with the present invention.

Because the polymerized protein (i.e. polymers of α-lactalbumin and β-lactoglobulin) retains a substantial amount of solubility in water, the cooled proteinaceous material, after formation of the polymerized protein, is microfiltered to separate the polymerized protein from any κ-casein macropeptide that is present in the cooled proteinaceous material. Beneficially, the κ-casein macropeptide, including glycomacropeptide, does not polymerize under the alkaline heat treatment regimen that is applied to the fluid proteinaceous material. Instead, the κ-casein macropeptide that has undergone the alkaline heat treatment remains soluble in water and is not polymerized. Indeed, the κ-casein macropeptide, after the alkaline heat treatment step, exists as oligomers that may be beneficially separated from the polymerized protein by microfiltration.

Following microfiltration, polymerized protein predominantly, if not fully, remains in the microfiltration retentate and may be dried to produce polymerized protein powder. On the other hand, κ-casein macropeptide, including glycomacropeptide, passes through the microfiltration membrane, and the resulting microfiltration permeate preferably contains most (at least about 70 weight percent), more preferably primarily all (at least about 80 weight percent), still more preferably predominantly all (at least about 90 weight percent), and most preferably all, of the κ-casein macropeptide originally present in the fluid proteinaceous material. Likewise, the resulting microfiltration permeate preferably contains most (at least about 70 weight percent), more preferably primarily all (at least about 80 weight percent), still more preferably predominantly all (at least about 90 weight percent), and most preferably all, of the glycomacropeptide originally present in the fluid proteinaceous material. Acid may then be added to the microfiltration retentate to give the microfiltration retentate a pH of about 5.5 to about 6.5, and preferably a pH of about 6 to about 6.5.

The acidified microfiltration retentate may then be concentrated by ultrafiltration to produce an ultrafiltration retentate and an ultrafiltration permeate. The ultrafiltration permeate contains water along with most, if not all, of the minerals, lactose, and ash originally present in the fluid proteinaceous material. The ultrafiltration retentate, on the other hand, is rich in κ-casein macropeptide, and consequently may be dried to produce a κ-casein macropeptide-enriched powder (also referred to herein as “κ-casein macropeptide powder” and as “CMP powder”).

The ultrafiltration retentate preferably contains most (at least about 70 weight percent), more preferably primarily all (at least about 80 weight percent), still more preferably predominantly all (at least about 90 weight percent), and most preferably all, of the κ-casein macropeptide and glycomacropeptide originally present in the fluid proteinaceous material. Furthermore, the concentrations of both κ-casein macropeptide and glycomacropeptide in the ultrafiltration retentate, based upon the total weight of protein in the ultrafiltration retentate, will typically be at least about two times, preferably at least about three times, and more preferably at least about four times, greater than the concentrations of κ-casein macropeptide and glycomacropeptide, respectively, originally present in the fluid proteinaceous material, based upon the total weight of protein in the fluid proteinaceous material.

Additionally, the ultrafiltration retentate, based on the total protein weight of the ultrafiltration retentate, will typically contain at least about 35 weight percent glycomacropeptide, preferably contains at least about 45 weight percent glycomacropeptide, and more preferably contains at least about 48 weight percent glycomacropeptide. Furthermore, the ultrafiltration retentate, based on the total protein weight of the ultrafiltration retentate, will typically contain at least about 40 weight percent κ-casein macropeptide, preferably contains at least about 45 weight percent κ-casein macropeptide, and more preferably contains at least about 50 weight percent κ-casein macropeptide.

Continuing, the κ-casein macropeptide-enriched powder preferably contains most (at least about 70 weight percent), more preferably primarily all (at least about 80 weight percent), still more preferably predominantly all (at least about 90 weight percent), and most preferably all, of the κ-casein macropeptide and glycomacropeptide originally present in the fluid proteinaceous material. Furthermore, the concentrations of κ-casein macropeptide and glycomacropeptide in the κ-casein macropeptide-enriched powder, based upon the total weight of protein in the κ-casein macropeptide-enriched powder, will typically be at least about two times greater, preferably at least about three times greater, and more preferably at least about four times greater, than the concentrations of κ-casein macropeptide and glycomacropeptide, respectively, originally present in the fluid proteinaceous material, based upon the total weight of protein in the fluid proteinaceous material.

Additionally, the κ-casein macropeptide-enriched powder, based on the total protein weight of the κ-casein macropeptide-enriched powder, will typically contain at least about 40 weight percent κ-casein macropeptide, preferably contains at least about 45 weight percent κ-casein macropeptide, and more preferably contains at least about 50 weight percent κ-casein macropeptide. Also, the κ-casein macropeptide-enriched powder, based on the total protein weight of the κ-casein macropeptide-enriched powder, will typically contain at least about 35 weight percent glycomacropeptide, preferably contains at least about 45 weight percent glycomacropeptide, and more preferably contains at least about 48 weight percent glycomacropeptide. Throughout this document, the terms “protein” and “proteins” are to be understood as meaning proteinaceous constituents that are made up of amino acids. As used herein, the terms “κ-casein macropeptide,” “casein macropeptide,” “caseinomacropeptide,” and “CMP” each refer collectively to all forms of κ-casein macropeptide ranging from (1) phosphorylated, glycosylated CMP; to (2) non-phosphorylated, glycosylated CMP; to (3) phosphorylated, non-glycosylated CMP; and to (4) non-phosphorylated, non-glycosylated CMP. On the other hand, the terms “x-casein glycomacropeptide,” “casein glycomacropeptide,” “glycomacropeptide,” and “GMP” each refer collectively to (1) phosphorylated, glycosylated CMP and to (2) non-phosphorylated, glycosylated CMP. Additionally, as used herein, the term “non-CMP protein,” when used with respect to a proteinaceous substance (i.e.: a substance that contains protein), refers to all protein constituents or components of the proteinaceous substance, other than any “CMP” component(s) of the proteinaceous substance. Also, as used herein, the term “non-GMP protein,” when used with respect to a proteinaceous substance, refers to all protein constituents or components of the proteinaceous substance, other than any “GMP” component(s) of the proteinaceous substance.

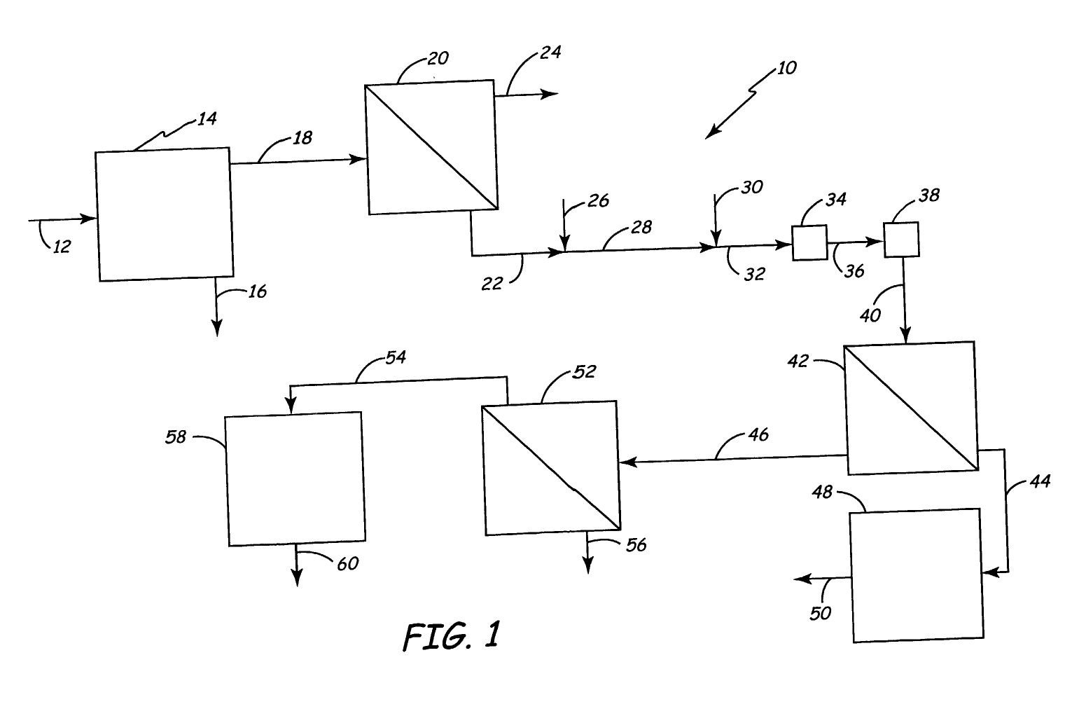

By way of example, the method of the present invention may be practiced using a process 10 that is depicted in FIG. 1. In the process 10, a proteinaceous feed 12, such as the previously-mentioned fluid proteinaceous material, may be, and preferably is, introduced into a separator 14 to remove fat 16 from the proteinaceous feed 12 and yield a reduced-fat proteinaceous feed 18. Operation of the separator 14 is preferably conducted to maximize removal of fat 16 from the proteinaceous feed 12, while maximizing retention of dissolved solids (esp. water-soluble protein) in the reduced-fat proteinaceous feed 18.

In one preferred form of the present invention, the proteinaceous feed 12 may be 34% whey protein concentrate formed by removing water in a first ultrafilter (not shown) from single strength whey to concentrate the dissolved solids (esp. water-soluble protein) of the single strength whey. The 34% whey protein concentrate contains about 34 weight percent total solids, based upon the total weight of the 34% whey protein concentrate. Thereafter, the 34% whey protein concentrate, as the proteinaceous feed 12, may be processed in the separator 14, which maybe a microfilter, for example, to remove fat 16 and yield the reduced-fat proteinaceous feed 18. Of course, as discussed below, a variety of materials other than 34% whey protein concentrate maybe employed as the proteinaceous feed 12.

The reduced-fat proteinaceous feed 18 that is discharged from the separator 14 maybe introduced into an ultrafiltration unit 20 (also referred to herein as “UF 1” and as the “ultrafilter 20”) that separates the reduced-fat proteinaceous feed 18 into an ultrafiltration retentate 22 (also referred to herein as “UF 1 retentate”) and an ultrafiltration permeate 24 (also referred to herein as “UF 1 permeate”). The UF 1 retentate may optionally be diafiltered in the ultrafilter 20 to yield the ultrafiltration retentate 22 as diafiltration retentate (not shown) and to yield the ultrafiltration permeate 24 as diafiltration permeate (not shown).

The step of ultrafiltering the reduced-fat proteinaceous feed 18 in the ultrafilter 20 is useful for separating lactose, ash, and water into the UF 1 permeate (or in the diafiltered form of the UF 1 permeate), while simultaneously concentrating desirable dissolved proteins (such as κ-casein macropeptide, α-lactalbumin, and/or β-lactoglobulin) in the UF 1 retentate (or in the diafiltered form of the UF 1 retentate). As an alternative, the proteinaceous feed 12 may bypass the separator 14 and be substituted in place of the reduced-fat proteinaceous feed 18 as the feed to the ultrafilter 20. However, the reduced-fat proteinaceous feed 18 is preferably the feed to the ultrafilter 20, since the preliminary step of removing fat from the proteinaceous feed 12 in the separator 14 generally enhances the rate at which the ultrafiltration permeate 24 is produced in the ultrafilter 20.

As noted above, one preferred form of the present invention employs 34% whey protein concentrate as the proteinaceous feed 12. Microfiltration of the 34% whey protein concentrate in the separator 14 (here, a microfilter) yields the reduced-fat proteinaceous feed 18. This reduced-fat proteinaceous feed 18 (derived by microfiltering 34% whey protein concentrate) may be filtered in the ultrafilter 20 to yield the UF 1 retentate (or in the diafiltered form of the UF 1 retentate) as whey protein isolate.

For all applications of ultrafiltration that are described herein, the term “diafiltration” is used as shorthand terminology for the conventional practice of adding additional water, preferably water with a low amount of total solids such as reverse osmosis water, to the ultrafiltration retentate during the ultrafiltration process. This addition of water to the ultrafiltration retentate further assists with passage of material through the ultrafiltration membrane into the ultrafiltration permeate and consequently helps minimize the concentration of solids, that are capable of passing through the ultrafiltration filtration membrane, in the resulting ultrafiltration retentate. Consequently, as used herein (including, but not limited to, the claims), the terms “ultrafiltration retentate” and “ultrafiltration permeate” are to be understood as optionally also referring to diafiltration retentate and diafiltration permeate, respectively, that result from addition of diafiltration water to the ultrafiltration retentate during ultrafiltration.

Similarly, for all applications of microfiltration that are described herein, the term “diafiltration” is used as shorthand terminology for the conventional practice of adding additional water, preferably water with a low amount of total solids such as reverse osmosis water, to the microfiltration retentate during the microfiltration process. This addition of water to the microfiltration retentate further assists with passage of material through the microfiltration membrane into the microfiltration permeate and consequently helps minimize the concentration of solids, that are capable of passing through the microfiltration filtration membrane, in the resulting microfiltration retentate. Consequently, as used herein (including, but not limited to, the claims), the terms “microfiltration retentate” and “microfiltration permeate” are to be understood as optionally also referring to diafiltration retentate and diafiltration permeate, respectively, that result from addition of diafiltration water to the microfiltration retentate during micro filtration.

The proteinaceous feed 12, as indicated by reference to the proteinaceous material that may serve as the proteinaceous feed 12, may contain any or all of κ-casein macropeptide, α-lactalbumin, and/or β-lactoglobulin, but more preferably includes κ-casein macropeptide, and at least α-lactalbumin or β-lactoglobulin, and still more preferably includes κ-casein macropeptide, α-lactalbumin, and β-lactoglobulin. The κ-casein macropeptide preferably includes glycomacropeptide, but may permissibly, and typically will, include other κ-casein macropeptides in addition to glycomacropeptide. One suitable example of the proteinaceous feed 12 is fluid whey from the production of cheese. Preferably, the fluid whey that may be used as the proteinaceous material 12 is sweet cheese whey, since sweet cheese whey is known to contain a significant amount of κ-casein macropeptide, such as glycomacropeptide, whereas acid whey may contain little, if any, κ-casein macropeptide.

In addition to, or as an alternative to, fluid whey, other non-exhaustive examples of the proteinaceous feed 12, or of components of the proteinaceous feed 12, include whey protein concentrate (at any concentration, such as 34% whey protein concentrate or 80% whey protein concentrate, for example), whey protein isolate (at any concentration, such as 34% whey protein isolate or 80% whey protein isolate, for example), or any of these in any combination. Any whey-based material(s) included in, or as, the proteinaceous feed 12, may have (1) an “as-produced” content of fat, water, lactose, minerals, and/or ash or (2) a reduced content of fat, water, lactose, minerals, and/or ash. Furthermore, any whey-based material(s) included in, or as, the proteinaceous feed 12 may be powdered or dried whey materials that are reconstituted when incorporated in the proteinaceous feed 12.

Any dairy material, such as full fat milk, reduced-fat milk, skim milk, reconstituted powdered or dried milk, or any of these in any combination may be incorporated in place of or in any combination with any of the aforementioned whey-based material(s) in the proteinaceous feed 12. However, the proteinaceous feed 12 preferably includes only sweet cheese whey or whey-based materials that are derived from sweet cheese whey to maximize the κ-casein macropeptide content and the glycomacropeptide content of the proteinaceous feed 12.

Additionally, any whey or whey-based material that is included in the proteinaceous feed 12 will typically be derived from milk that is produced by ruminants, and any milk that is included in the proteinaceous feed 12 will typically be produced by ruminants. As used herein, the term “ruminant” means an even-toed, hoofed animal that has a complex 3- or 4-chamber stomach, where the animal typically rechews material that it has previously swallowed. Some non-exhaustive examples of ruminants include cattle, sheep, goats, buffalo, oxen, musk ox, llamas, alpacas, guanicas, deer, reindeer, bison, antelopes, camels, and giraffes.

In the process 10, when the proteinaceous feed 12 is based upon sweet cheese whey, the ultrafiltration retentate 22 will typically contain about 25 weight percent protein (as total protein), based upon the total weight of the ultrafiltration retentate 22; will typically have a pH in the order of about 6; and will contain κ-casein macropeptide (including glycomacropeptide), α-lactalbumin, β-lactoglobulin, and some fat, though the fat concentration will often be relatively low. After production, the ultrafiltration retentate 22 may optionally be diluted with added water 26 to produce a diluted intermediate 28 with a protein content of less than about eight weight percent, based upon the total weight of the diluted intermediate 28. More preferably, sufficient water 26 is added to give the diluted intermediate 28 a protein concentration of about four weight percent, based upon the total weight of the diluted intermediate 28. Addition of water 26 to produce the diluted intermediate 28 is desirable to enhance separation of protein molecules during subsequent heating that occurs in the process 10 and help prevent protein coagulation as an intractable gel.

The pH of the diluted intermediate 28 is then adjusted, if necessary, by adding an alkaline agent 30 to the diluted intermediate 28 (or to the ultrafiltration retentate 22 if the protein concentration in the ultrafiltration retentate 22 is sufficiently dilute) to form an alkaline intermediate 32 with a pH greater than about 7, preferably about 8 or more, and more preferably about 8. Of course, no alkaline agent 30 need be added if the pH of the diluted intermediate 28 (or of the ultrafiltration retentate 22 if the protein concentration in the ultrafiltration retentate 22 is sufficiently dilute) is already greater than about 7, preferably about 8 or more, and more preferably about 8. The alkaline agent 30 may be any non-toxic, food grade, alkaline material that is capable of yielding the alkaline intermediate 32 with the requisite pH.

Some non-exhaustive examples of suitable alkaline agents 30 include magnesium oxide, calcium oxide, sodium hydroxide, potassium hydroxide, calcium hydroxide, and any of these in any combination. Sodium hydroxide is a preferred form of the alkaline agent 30, because sodium hydroxide is relatively inexpensive and is readily available. The alkaline agent 30 may be introduced in either solid form or in aqueous solution. However, the alkaline agent 30 is preferably introduced as an aqueous solution, because aqueous solutions are easier to meter and will tend to mix more quickly and uniformly with the diluted intermediate 28 (or with the ultrafiltration retentate 22 if the protein concentration in the ultrafiltration retentate 22 is sufficiently dilute).

After any pH adjustment to the diluted intermediate 28, the alkaline intermediate 32 may be heated, by adding culinary grade steam 34, for example, to form a heated intermediate 36. The heated intermediate 36 should attain an elevated temperature of at least about 175° F., more preferably at least about 180° F., and still more preferably at least about 190° F. to support the desired polymerization of any α-lactalbumin and any β-lactoglobulin originally present in the heated intermediate 36. After being heated, the heated intermediate 36 maybe held for the holding period, prior to being cooled, to form a cooled protein solution 40.

The holding period for the heated intermediate 36 may range from as little as about one minute up to about ten minutes to about fifteen minutes, or more, to support the desired polymerization of any α-lactalbumin and any β-lactoglobulin originally present in the heated intermediate 36. In one preferred form of the present invention, the holding period is at least about two minutes. In another preferred form of the present invention, the holding period is at least about five minutes, or more. In yet another preferred form of the present invention, the holding period is about eight to about twelve minutes, and more preferably about ten minutes.

Polymerization of α-lactalbumin and/or β-lactoglobulin is discussed throughout this document. Polymerization of α-lactalbumin yields polymers of α-lactalbumin. Likewise, polymerization of β-lactoglobulin yields polymers of β-lactoglobulin. However, solutions that are subject to polymerization in accordance with the present invention will often contain, and preferably do contain, both α-lactalbumin and β-lactoglobulin.

On the other hand, polymerization of α-lactalbumin and β-lactoglobulin, in accordance with the present invention, will typically yield a mixture of polymers that includes (1) polymers with both α-lactalbumin groups and β-lactoglobulin groups along with (2) some polymers of α-lactalbumin and (3) some polymers of β-lactoglobulin. However, depending upon a variety of conditions, including but not limited to, the concentrations of α-lactalbumin and β-lactoglobulin, the temperature of the solution during the polymerization reaction, the duration of the polymerization reaction, polymerization of α-lactalbumin and β-lactoglobulin, in accordance with the present invention, may, at least theoretically, yield only one, any two, or any three of the following: (1) polymers with both α-lactalbumin groups and β-lactoglobulin groups, (2) polymers of α-lactalbumin, and/or (3) polymers of β-lactoglobulin. Therefore, as used herein, the term “polymers of α-lactalbumin and β-lactoglobulin” (and “polymers of β-lactoglobulin and α-lactalbumin”), unless stated otherwise herein, is to be understood as meaning “(1) polymers that include both α-lactalbumin groups and β-lactoglobulin groups, (2) polymers of α-lactalbumin, (3) polymers of β-lactoglobulin, or any combination of any of these polymers.”

Continuing, the heated intermediate 36 may be held at about the elevated temperature during the holding period. However, as another permissible alternative, the heated intermediate 36 may be held, without additional heating during the holding period, so that the temperature of the heated intermediate 36 slowly falls from the elevated temperature, prior to cooling of the heated intermediate 36 to form the cooled protein solution 40. However, even absent additional heating to hold the heated intermediate 36 at the elevated temperature during the holding period, the heated intermediate 36 is preferably held in an insulated vessel (not shown) during the holding period to slow the drop in temperature of the heated intermediate 36 from the elevated temperature during the holding period.

As an alternative to the culinary steam 34, the alkaline intermediate 32 may alternatively be heated by passing the alkaline intermediate 32 through a conventional heat exchanger (not shown), such as a tube-in-shell heat exchanger or a plate heat exchanger. As another example, the alkaline intermediate may be passed through a conventional high temperature, short time (HTST) plate heat exchanger.

The choice of the elevated temperature that the heated intermediate 36 attains and the duration selected for the holding period will typically effect the recovery (preservation) of κ-casein macropeptide, such as glycomacropeptide, in streams derived from the cooled protein solution 40, conversion of β-lactoglobulin and/or α-lactalbumin to polymers of β-lactoglobulin and α-lactalbumin, and recovery of polymerized proteins in streams derived from the cooled protein solution 40. In general, increasing the elevated temperature and/or increasing the holding period duration will tend to reduce recovery (preservation) of κ-casein macropeptide, such as glycomacropeptide, in streams derived from the cooled protein solution 40, increase conversion of β-lactoglobulin and/or α-lactalbumin to polymers of β-lactoglobulin and α-lactalbumin, and increase recovery of polymerized proteins in streams derived from the cooled protein solution 40. On the other hand, decreasing the elevated temperature and/or decreasing the holding period duration will tend to increase recovery (preservation) of κ-casein macropeptide, such as glycomacropeptide, in streams derived from the cooled protein solution 40, decrease conversion of β-lactoglobulin and/or α-lactalbumin to polymers of β-lactoglobulin and α-lactalbumin, and decrease recovery of polymerized proteins in streams derived from the cooled protein solution 40.

After the holding period, the heated intermediate 36 may be cooled, preferably within about 30 seconds or less, to a temperature of less than about 140° F. and preferably to a temperature of about 120° F., or less, by adding culinary cooling water 38, for example, to form the cooled protein solution 40. In one preferred form of the present invention, the heated intermediate 36 is cooled, after the holding period, to a temperature of about 70° F. As an alternative to adding culinary cooling water 38, the heated intermediate 36 may be passed through a conventional heat exchanger (not shown) to cool the heated intermediate 36 and form the cooled protein solution 40.

Alternatively, though not depicted in FIG. 1, instead of adding the alkaline agent 30 to the diluted intermediate 28, the diluted intermediate 28 may be heated followed by adding the alkaline agent 30 to the heated form of the diluted intermediate 28 to form the heated intermediate 36. As another alternative, the diluted intermediate 28 may be heated and the alkaline agent 30 may be added during heating of the diluted intermediate 28 to form the heated intermediate 36. Each of these approaches are satisfactory alternatives to the alkaline agent 30 addition and heating approach depicted in FIG. 1, since pH has been found to have little, if any, influence on the tendency of κ-casein macropeptide, such as glycomacropeptide, to become denatured under the heating conditions (temperature and duration) described above for the heated intermediate 36.

Also, denaturization (polymerization) of α-lactalbumin and β-lactoglobulin is a stronger function of heat as opposed to pH, though higher alkaline pHs (esp. pHs of about 8 or more) tend to enhance the denaturing effects of heat on α-lactalbumin and β-lactoglobulin. Preferably, however, the alkaline agent 30 is added to the diluted intermediate 28 and the alkaline intermediate 32 is thereafter heated, since pH adjustment before heating is less complicated, allows for a more uniform pH in the solution before heating, and would be expected to reduce the overall heating time required to produce a desired degree of α-lactalbumin and β-lactoglobulin denaturization, and consequently a desired degree conversion of α-lactalbumin and/or β-lactoglobulin to polymers of α-lactalbumin and β-lactoglobulin. Much of any α-lactalbumin and/or any β-lactoglobulin originally present in the heated intermediate 36 is polymerized to yield the cooled protein solution 40 that contains polymerized protein (i.e.: polymers of α-lactalbumin and β-lactoglobulin). Preferably at least about 70 weight percent, more preferably at least about 80 weight percent, and still more preferably at least about 90 weight percent, of any α-lactalbumin and any β-lactoglobulin that is originally present in the heated intermediate 36 is transformed into polymerized protein. Additionally, the cooled protein solution 40 contains κ-casein macropeptide, such as glycomacropeptide, (if originally present in the proteinaceous feed 12 and consequently present in the ultrafiltration retentate 22) that has not been polymerized, since it is known that the pH conditions prescribed for the heated intermediate 36 and the heating temperature and duration for the heated intermediate 36 tend to support only minor, if any, denaturization or polymerization of κ-casein macropeptide, such as glycomacropeptide. Furthermore, the cooled protein solution 40 includes any fat present in the ultrafiltration retentate 22 and water, including any water added as a result of addition of the dilution water 26, the alkaline agent 30, the culinary grade steam 34, and the culinary cooling water 38.

The primary proteinaceous components of the cooled protein solution 40 are the κ-casein macropeptide, such as glycomacropeptide, if present, and the polymerized protein that includes polymers of α-lactalbumin and β-lactoglobulin, along with polymerized forms of any other polymerizable proteins originally present in the heated intermediate 36. Thus, κ-casein macropeptide, the polymerized protein, and fat will typically constitute all, or predominantly all, of the solids present in the cooled protein solution 40, if κ-casein macropeptide, other soluble proteins, such as α-lactalbumin and β-lactoglobulin, and fat constitute all, or predominantly all, of the solids present in the ultrafiltration retentate 22. The molecules of the polymerized protein, though substantially, or even fully, soluble in water, are relatively large molecules. On the other hand, any κ-casein macropeptide, such as glycomacropeptide, present in the cooled protein solution 40 will be in oligomeric form as relatively small molecules, at least as compared to the polymerized protein.

The cooled protein solution 40 may be microfiltered (and optionally diafiltered) in a microfilter 42 to separate κ-casein macropeptide, such as glycomacropeptide, from the polymerized protein. The cooled protein solution 40, still at a pH of greater than about 7, preferably about 8 or more, and more preferably about 8, may be processed in the microfilter 42 to form a microfiltration retentate 44 and a microfiltration permeate 46. The temperature of the cooled protein solution 40 during microfiltration in the microfilter 42 may generally range from about 40° F. to less than about 140° F., and is preferably about 70° F.

Microfiltration of the cooled protein solution 40 preferably causes essentially all (at least about 95 weight percent), and more preferably all of the polymerized protein to exit the microfilter 42 as part of the microfiltration retentate 44. On the other hand, microfiltration of the cooled protein solution 40 preferably causes most (at least about 70 weight percent), more preferably primarily all (at least about 80 weight percent), still more preferably predominantly all (at least about 90 weight percent), and most preferably all, of the κ-casein macropeptide and glycomacropeptide originally present in the cooled protein solution 40 to exit the microfilter 42 as part of the microfiltration permeate 46. Preferably, diafiltration is employed in the microfilter 42 to maximize recovery of κ-casein macropeptide and glycomacropeptide in the microfiltration permeate 46, while enhancing recovery of polymerized protein in the microfiltration retentate 44. In addition to the polymerized protein, most, if not all, of the small amount of fat present in the cooled protein solution 40 will typically remain with the polymerized protein in the microfiltration retentate 44.

The microfiltration retentate 44 may then be transferred to a dryer, such as a spray dryer 48, where the microfiltration retentate 44 may be spray dried to form a polymerized protein powder 50. The polymerized protein powder 50 primarily consists of the polymerized protein, such as polymers of α-lactalbumin and β-lactoglobulin, from the cooled protein solution 40. Where the polymerized protein powder 50 is derived from sweet cheese whey (as the proteinaceous feed 12), the polymerized protein content of the polymerized protein powder that includes polymers of α-lactalbumin and β-lactoglobulin has surprisingly been found to have a substantial amount, such as at least about 50 percent, of the solubility in water, at similar weight-based concentrations and solution temperatures, that is possessed by the α-lactalbumin and β-lactoglobulin of the proteinaceous feed 12 that have not been polymerized or subjected to the alkaline heat treatment step of the present invention.

By way of comparison, the pH of the cooled protein solution 40 may optionally be adjusted to an acidic pH, such as to a pH of about 6, prior to microfiltration of the cooled protein solution 40. Nonetheless, when the proteinaceous feed 12 is sweet cheese whey, it has been surprisingly found that the polymerized protein powder 50 has a substantially greater solubility in water, at similar weight percent concentrations and solution temperatures, when the pH of the cooled protein solution 40 remains alkaline, as compared to the solubility in water of polymerized protein powder 50 produced following microfiltration of the cooled protein solution 40 having an acidic pH.

Additionally, solutions of the powdered protein powder 50, at similar weight-based concentrations and solution temperatures, are significantly more viscous in water when the cooled protein solution 40 that is processed in the microfilter 42 has an alkaline pH, as compared to when the cooled protein solution 40 that is processed in the microfilter 42 has a pH of about 7 or less, such as a pH of about 6. For example, when the cooled protein solution 42 is given a pH of about 6.0, such as a pH of 6.3, prior to being processed in the microfilter 42, the resulting polymerized protein powder 50, when dissolved in water to form a 10 weight percent aqueous solution of the polymerized protein powder 50, based upon the total weight of the aqueous solution, generally will have a Brookfield viscosity at about room temperature (about 72° F.) of at least about 6,000 centipoise, and preferably at least about 8,000 centipoise.

On the other hand, when the cooled protein solution 42 has an alkaline pH, such as a pH of about 8, prior to being processed in the microfilter 42, the resulting polymerized protein powder 50, when dissolved in water to form a 10 weight percent aqueous solution of the polymerized protein powder 50, based upon the total weight of the aqueous solution, generally will have a Brookfield viscosity at about room temperature (about 72° F.) of at least about 10,000 centipoise, and preferably at least about 11,000 centipoise. Nonetheless, even when the cooled protein solution 40 that is processed in the microfilter 42 has a pH of about 7 or less, such as an acidic pH of about 6, solutions of the polymerized protein powder 50 are many times more viscous, such as thousands of times more viscous, than solutions of powdered whey protein concentrate or powdered whey protein isolate that have not been processed in accordance with the present invention.

Thus, at similar weight-based concentrations and solution temperatures, the polymerized protein powder 50 forms much more viscous solutions in water than either powdered whey protein concentrate or powdered whey protein isolate that each contain only non-polymerized forms of α-lactalbumin and β-lactoglobulin. This statement holds true no matter if the cooled protein solution 40 that is processed in the microfilter 42 has a neutral pH of about 7; an acidic pH, such as a pH of about 6.3, for example; or an alkaline pH, such as a pH of about 8, for example.

Additionally, beyond creating more viscous solutions, the polymerized protein powder 50, when combined with water, forms clear weak gels and has been found to have a significant amount of water binding capability. For example, when analyzed according to the LCE gelation test referenced in Example 3 below, an aqueous solution with a concentration of 10 weight percent of the polymerized protein powder 50 in reverse osmosis water, based upon the total weight of the aqueous solution, preferably has a water holding capacity of at least about 75 percent without addition of salt, and a water holding capacity of about 100 percent with the addition of salt. More preferably, when analyzed according to the LCE gelation test, an aqueous solution with a concentration of 10 weight percent of the polymerized protein powder 50 in reverse osmosis water, based upon the total weight of the aqueous solution, has a water holding capacity, both with and without any salt addition, has a water holding capacity of about 100 percent,

The microfiltration permeate 46 that exits the microfilter 42 preferably contains most, more preferably predominantly all, still more preferably essentially all, and most preferably all of the κ-casein macropeptide, such as glycomacropeptide, originally present in the cooled protein solution 40. However, the microfiltration permeate 46 also includes a relatively large amount of water, since the microfiltration permeate 46 contains most of the water originally present in the cooled protein solution 40. Therefore, the microfiltration permeate 46 may be dewatered, preferably using an ultrafilter 52, to remove a substantial amount of the water present in the microfiltration permeate 46 and thereby minimize the amount of water subsequently needing to be removed by drying, such as by spray drying.

Both an ultrafiltration retentate 54 and an ultrafiltration permeate 56 are produced by ultrafiltering the microfiltration permeate 46 in the ultrafilter 52. The ultrafiltration retentate 54 preferably contains most (at least about 70 weight percent), more preferably primarily all (at least about 80 weight percent), still more preferably predominantly all (at least about 90 weight percent), and most preferably all, of the κ-casein macropeptide, such as glycomacropeptide, originally present in the microfiltration permeate 46, whereas the ultrafiltration permeate 56 contains most, if not essentially all, of the water originally present in the microfiltration permeate 46.

After concentration of the κ-casein macropeptide, such as glycomacropeptide, in the ultrafiltration retentate 54, the ultrafiltration retentate 54 may be directed to a spray dryer 58 that removes any water originally present in the ultrafiltration retentate 54 and forms a κ-casein macropeptide-enriched powder 60, such as a glycomacropeptide-enriched powder. The κ-casein macropeptide that forms the majority of the κ-casein macropeptide-enriched powder 60 retains the water solubility characteristics of the κ-casein macropeptide originally present in the proteinaceous feed 12, despite the heat treatment undergone by the intermediate 36, no matter the pH of the intermediate 36 during heat treatment of the intermediate 36.

κ-casein macropeptide, such as glycomacropeptide, forms the majority of the κ-casein macropeptide-enriched powder 60. This κ-casein macropeptide of the powder 60 has a high degree of solubility in water. Therefore, the κ-casein macropeptide-enriched powder 60 may be employed as a rich source of water-soluble κ-casein macropeptide, such as water-soluble glycomacropeptide, in foods destined for human consumption and in feeds destined for animal consumption. This isolation of a high purity κ-casein macropeptide source with excellent solubility characteristics consequently provides a flexible and easily incorporated source of κ-casein macropeptide, such as glycomacropeptide, in food and feeds, where the beneficial biological activities of the κ-casein macropeptide may be harnessed for the good of both humans and animals alike.

The weight ratio of κ-casein macropeptide, such as the weight ratio of glycomacropeptide, to the collective weight of any β-lactoglobulin and any α-lactalbumin in the κ-casein macropeptide-enriched powder 60 of the present invention may be varied in a wide range, depending upon whether the elevated temperature and the holding period are selected to increase recovery (preservation) of κ-casein macropeptide in streams derived from the cooled protein solution 40 or are instead selected to recovery of polymerized proteins in streams derived from the cooled protein solution 40. When the elevated temperature and the holding period are selected to increase recovery (preservation) of κ-casein macropeptide, such as glycomacropeptide, in streams derived from the cooled protein solution 40 this will typically have the effect of also reducing the conversion of β-lactoglobulin and α-lactalbumin to polymerized protein.

Consequently, when the elevated temperature and the holding period are selected to increase recovery (preservation) of κ-casein macropeptide, the weight ratio of κ-casein macropeptide to the collective weight of any β-lactoglobulin and any α-lactalbumin in the κ-casein macropeptide-enriched powder 60 (referred to herein as the “κ-casein macropeptide enrichment ratio”) will also increase. When κ-casein macropeptide enrichment is desired over polymerized protein production and recovery, the microfilter 42 and the ultrafilter 52 may be configured and operated to provide κ-casein macropeptide-enriched powder 60 that exhibits a desired κ-casein macropeptide enrichment ratio, such as at least about 0.55, preferably at least about 0.7, more preferably at least about 1, still more preferably at least about 2, and even more preferably at least about 3, to increase the value of the κ-casein macropeptide-enriched powder 60 in terms of κ-casein macropeptide.

Correspondingly, when the elevated temperature and the holding period are selected to increase recovery (preservation) of glycomacropeptide, the weight ratio of to the collective weight of any β-lactoglobulin and any α-lactalbumin in the glycomacropeptide-enriched powder (referred to herein as the “glycomacropeptide enrichment ratio”) will also increase. When glycomacropeptide enrichment is desired over polymerized protein production and recovery, the microfilter 42 and the ultrafilter 52 may be configured and operated to provide glycomacropeptide-enriched powder 60 that exhibits a desired glycomacropeptide enrichment ratio, such as at least about 0.55, preferably at least about 0.7, more preferably at least about 1, still more preferably at least about 2, and even more preferably at least about 3, to increase the value of the glycomacropeptide-enriched powder in terms of glycomacropeptide.

When the elevated temperature and the holding period are selected to increase the conversion of β-lactoglobulin and α-lactalbumin to polymerized protein, this will typically have the effect of also reducing recovery (preservation) of i<-casein macropeptide in streams derived from the cooled protein solution 40. Consequently, when the elevated temperature and the holding period are selected to increase the conversion of β-lactoglobulin and α-lactalbumin to polymerized protein, the weight ratio of κ-casein macropeptide to the collective weight of any β-lactoglobulin and any α-lactalbumin in the κ-casein macropeptide-enriched powder 60 (i.e.: the “κ-casein macropeptide enrichment ratio”) will typically be expected to decrease. When the microfilter 42 and the ultrafilter 52 are configured and operated to favor polymerized protein production and recovery enhancement over κ-casein macropeptide enrichment, the κ-casein macropeptide-enriched powder 60 may exhibit a κ-casein macropeptide enrichment ratio of less than about 0.7, preferably less than about 0.55, more preferably less than about 0.4, and still more preferably less than about 0.2.

When the elevated temperature and the holding period are selected to increase the conversion of β-lactoglobulin and α-lactalbumin to polymerized protein, this will typically have the effect of also reducing recovery (preservation) of glycomacropeptide in streams derived from the cooled protein solution 40. Consequently, when the elevated temperature and the holding period are selected to increase the conversion of β-lactoglobulin and α-lactalbumin to polymerized protein, the weight ratio of glycomacropeptide to the collective weight of any β-lactoglobulin and any α-lactalbumin in the glycomacropeptide-enriched powder (i.e.: the “glycomacropeptide enrichment ratio”) will typically be expected to decrease. When the microfilter 42 and the ultrafilter 52 are configured and operated to favor polymerized protein production and recovery enhancement over glycomacropeptide enrichment, the glycomacropeptide-enriched powder may exhibit a κ-casein macropeptide enrichment ratio of less than about 0.7, preferably less than about 0.55, more preferably less than about 0.4, and still more preferably less than about 0.2 When increased recovery (preservation) of κ-casein macropeptide is desired, the κ-casein macropeptide-enriched powder 60 preferably has a total protein concentration of at least about 75 weight percent, and more preferably at least about 80 weight percent, based upon the total weight of the κ-casein macropeptide-enriched powder 60. Additionally, the κ-casein macropeptide-enriched powder 60, based on the total protein weight of the κ-casein macropeptide-enriched powder 60, will typically contain at least about 40 weight percent κ-casein macropeptide, but preferably contains at least about 45 weight percent κ-casein macropeptide and more preferably contains at least about 50 weight percent κ-casein macropeptide to increase the value of the κ-casein macropeptide-enriched powder 60 in terms of κ-casein macropeptide.

Correspondingly, when increased recovery (preservation) of glycomacropeptide is desired, the glycomacropeptide-enriched powder preferably has a total protein concentration of at least about 75 weight percent, and more preferably at least about 80 weight percent, based upon the total weight of the glycomacropeptide-enriched powder. Additionally, the glycomacropeptide-enriched powder, based on the total protein weight of the glycomacropeptide-enriched powder, will typically contain at least about 35 weight percent glycomacropeptide, but preferably contains at least about 45 weight percent glycomacropeptide and more preferably contains at least about 48 weight percent glycomacropeptide to increase the value of the glycomacropeptide-enriched powder 60 in terms of glycomacropeptide.

In the process 10, alkaline agent addition and/or acid agent addition are described for purposes of adjusting the pH of a particular stream, as desired, at particular points without the process 10. Other than for the alkaline agent and/or acid agent addition, as noted above, and water added during, or for purposes of, heating (as steam), cooling, diluting, or diafiltering a particular stream within the process 10, no other chemical agents, no precipitation agents or aids, and no filtration aids need be added to any stream within the process 10. Preferably, other than for the noted alkaline agent and/or acid agent addition and the addition of water for purposes of heating, cooling, diluting, or diafiltering a particular stream within the process 10, no other chemical agents, no precipitation agents or aids, and no filtration aids are added to the proteinaceous feed 12, the reduced-fat proteinaceous feed 18, the UF 1 retentate 22, the diluted intermediate 28, the alkaline intermediate 32, the heated intermediate 36, the cooled protein solution 40, or any stream that is processed in the microfilter 42 or in either of the ultrafilters 20, 52.

Also, no salt content adjustment is typically needed for any stream within the process 10, though any water added during, or for purposes of, heating, cooling, diluting, or diafiltering a particular stream within the process 10 is preferably water with a low salt content, such as reverse osmosis water. Preferably, no salt content adjustment is conducted on the proteinaceous feed 12, the reduced-fat proteinaceous feed 18, the UF 1 retentate 22, the diluted intermediate 28, the alkaline intermediate 32, the heated intermediate 36, the cooled protein solution 40, or any stream that is processed in the microfilter 42 or in either of the ultrafilters 20, 52, with the exception that any water added during or for purposes of heating, cooling, diluting, or diafiltering a particular stream within the process 10 is preferably water with a low salt content, such as reverse osmosis water.

The ultrafilters 20 and 52 may each employ an ultrafiltration membrane with a molecular weight cut-off (also referred to as “MWCO”) of approximately 3000 Daltons, since water, lactose, minerals, and ash typically have molecular weights on the order of about 1000 Daltons or less, whereas native and soluble proteins, such as κ-casein macropeptide (such as glycomacropeptide), α-lactalbumin, and β-lactoglobulin typically have molecular weights greater than about 3000 Daltons. One suitable ultrafiltration membrane with a MWCO of approximately 3000 Daltons is available from Koch Membrane Systems of Wilmington, Mass. as an ABCOR® ultrafiltration membrane. Other suitable ultrafiltration membranes with MWCOs of approximately 3000 Daltons are available from PTI Advanced Filtration, Inc. of San Diego, Calif.; from Synder Filtration of Vacaville, Calif.; and from Osmonics, Inc. of Minnetonka, Minn. Suitable ceramic ultrafiltration membranes are available from Ceraver of France and from U.S. Filter Corporation of Rockford, Ill. Additionally, suitable zirconium-coated ultrafiltration membranes are available from Rhone-Poulenc of France.

The performance of an ultrafiltration membrane, as measured by the flow rate of the ultrafiltration permeate, is primarily dictated by the surface area of the ultrafiltration membrane, by the characteristics of the feed to the ultrafiltration membrane, and by the feed side pressure maintained on the ultrafiltration membrane. The flow rate of ultrafiltration permeate from the ultrafilters 20 and 52 is thus controlled by the performance of the ultrafiltration membrane that is used in the ultrafilters 20 and 52, respectively. Similar comments apply when diafiltration is practiced using the ultrafilters 20 and 52.

The back pressure may be controlled on the retentate from the ultrafilters 20 and 52 to maintain a particular degree of ultrafiltration retentate concentration relative to the ultrafiltration feed. Alternatively, the flow rate of the retentate from the ultrafilters 20 and 52 may be controlled at a particular ratio with respect to the flow rate of the permeate from the ultrafilters 20 and 52 by controlling the back pressure on the ultrafiltration retentate.

For the ultrafilters 20 and 52, the inlet pressure on the feed to the ultrafilter and the discharge pressure on the ultrafiltration retentate may be maintained within a broad range of pressures to attain acceptable ultrafiltration results, so long as the selected pressures do not cause degradation of the particular ultrafiltration membrane being used. For example, the inlet pressure on the feed to the ultrafilters 20 and 52 may be maintained from about 40 pounds per square inch (“psig”) to about 120 psig, and the discharge pressure on the ultrafiltration retentate from the ultrafilters 20 and 52 may generally be maintained from about 0 psig to about 60 psig, so long as the difference between the inlet pressure on the feed to the ultrafilters 20 and 52 and the discharge pressure on the ultrafiltration retentate is greater than 0 psig and preferably ranges from about 40 psig to about 80 psig. For example, it would be acceptable in the course of practicing the present invention to maintain an inlet pressure of about 80 psig on the feed to the ultrafilters 20 and 52, while maintaining a discharge pressure of about 20 psig on the ultrafiltration retentate coming from the ultrafilters 20 and 52. Similar comments apply when diafiltration is practiced using the ultrafilters 20 and 52.

The temperature of the feed to, and in, the ultrafilters 20 and 52 may generally be maintained in the range from about 40° F. to about 140° F., so long as the selected temperature does not cause degradation of the particular ultrafiltration membrane being used. For example, it would typically be acceptable to maintain the temperature of the feed to, and in, the ultrafilters 20 and 52 in the range from about 55° F. to about 120° F. during ultrafiltration. Similar comments apply when diafiltration is practiced using the ultrafilters 20 and 52.

One major purpose of the microfilter 42 is to separate polymerized protein present in the cooled protein solution 40 from any κ-casein macropeptide that is present in the cooled protein solution 40. Preferably, any polymerized protein present in the cooled protein solution 40 predominantly, if not fully, exits the microfilter 42 as part of the microfiltration retentate 44, and any κ-casein macropeptide present in the cooled protein solution 40 predominantly, if not fully, exits the microfilter 42 as part of the microfiltration permeate 46.

Increasing the number of membrane stages in the microfilter 42 will enhance the tendency for membrane transfer of κ-casein macropeptide, such as glycomacropeptide, to the microfiltration permeate 46 and will tend to enhance polymerized protein recovery in the microfiltration retentate 44. In this regard, the terms “microfilter” and “microfiltration” are to be broadly construed as referring to any separation mechanism, such as a filtration mechanism, that is capable of preferably permitting all or at least essentially all (at least about 95 weight percent), of the polymerized protein that is present in the feed to the processing unit, such as the microfilter 42, to be transferred into a first stream, such as the microfiltration retentate 44, and to preferably allow most (at least about 70 weight percent), more preferably primarily all (at least about 80 weight percent), still more preferably predominantly all (at least about 90 weight percent), and most preferably all, of the κ-casein macropeptide and glycomacropeptide present in the feed to the processing unit, such as the microfilter 42, to be transferred into a second stream, such as the microfiltration permeate 46.

The microfiltration membrane used in the microfilter 42 may be made of any suitable material that is capable of extended operating periods of at least about seven days and preferably at least about 30 days. Additionally, the material of the microfiltration membrane should be capable of substantially maintaining the pore size distribution and pore size range of the microfiltration membrane during operating periods of at least about seven days and preferably during operating periods of at least about 30 days. Though the terms “microfilter” and “microfiltration” are defined herein in terms of separation capability with respect to the polymerized protein and the κ-casein macropeptide, such as glycomacropeptide, that are present in the feed to the microfilter 42, the terms “microfilter” and “microfiltration” are also discussed herein in terms of the molecular weight cut-off (MWCO) of particular membranes.

Membrane MWCOs are characterized based on filtration of a clear aqueous solution that contains molecules of a particular size. For example, when a 1,000,000 Dalton MWCO membrane is used to filter a clear aqueous solution containing only molecules with a molecular weight of 1,000,000 Daltons, by definition, 95 weight percent of the molecules will be retained by the membrane while 5 weight percent of the molecules will pass through the membrane. Because the solution that is used to characterize the MWCO of the membrane contains only molecules with a molecular weight of approximately 1,000,000 Dalton, secondary membrane effects do not occur to a significant degree and therefore do not significantly affect the amount of the 1,000,000 Dalton molecules that are retained on the membrane.

Some examples of membranes that may serve as microfiltration membranes for the microfilter 42 in accordance with the present invention include those membranes having a MWCO ranging from approximately 500,000 Daltons to approximately 1,000,000 Daltons. Preferably, the membrane of the microfilter 42 has a MWCO greater than approximately 500,000 Daltons. Some examples of suitable microfiltration membrane materials for the microfilter 42 include polysulfone, polyvinyl difluoride (PVDF) and ceramic. Of these, PVDF and ceramic are preferred over polysulfone, and PVDF is preferred over ceramic.

PVDF is preferred over ceramic because ceramic membranes and seals associated with ceramic membranes have been found to require special care during operations. For example, ceramic membranes are relatively susceptible to cracking if temperature changes in the feed material to the membrane are made too quickly, due to differential expansion rates within the ceramic material. Additionally, the seals associated with ceramic membranes require delicate torquing during installation to prevent damage to the seals that may permit leakage through the seals.

Ceramic and PVDF are preferred over polysulfone because ceramic and PVDF each have a more uniform pore size distribution than polysulfone membranes. Filtration membranes typically have a distinctive range of pore sizes that may be affected by the membrane material and the technique used to form the pores in the membrane. Exemplary microfiltration structures that may be used in practicing the method of the present invention are the AF series of membrane structures with MWCOs ranging from approximately 500,000 Daltons to approximately 1,000,000 Daltons and a nominal pore diameter in the range of about 0.02 microns to less than about 0.2 microns, and preferably a nominal pore diameter of about 0.05 microns, that are available from PTI Advanced Filtration, Inc. of San Diego, Calif. The AF-1000 microfiltration membrane structure, which has an MWCO of about 1,000,000 Daltons, is an suitable example of an AF series membrane structure available from PTI Advanced Filtration. Other suitable ultrafiltration membranes with MWCOs ranging from approximately 500,000 Daltons to approximately 1,000,000 Daltons and a nominal pore diameter in the range of about 0.02 microns to less than about 0.2 microns, and preferably a nominal pore diameter of about 0.05 microns are available from Synder Filtration of Vacaville, Calif.

The pore size distribution and the range of pore sizes directly affects the separation characteristics of a particular filtration membrane. The pore size range and distribution of a particular membrane substantially determines the range and distribution of particle size(s) that pass through the membrane. A particular membrane is better able to differentiate between particle sizes and make a sharper particle size cut when the membrane has a more uniform pore size distribution.

Polysulfone membranes have a less uniform pore size distribution than PVDF membranes. On the other hand, ceramic membranes have a more uniform pore size than PVDF membranes. When practicing the microfiltration portion of the present invention, it is desirable to maintain a sharper cut-off point between particles and molecules that permeate through the membrane versus particles and molecules that are retained on the membrane. For this reason, ceramic and PVDF membranes are preferred over polysulfone membranes. However, PVDF membranes are preferred over ceramic membranes due to the previously noted operational problems that may arise when ceramic membranes are used.

As noted, the present invention may generally incorporate microfiltration membranes with MWCOs ranging from approximately 500,000 Daltons to approximately 1,000,000 Daltons. For example, the microfilter 42 may incorporate a membrane with a MWCO of about 500,000 Daltons. However, the microfilter 42 preferably incorporates a membrane with a MWCO greater than approximately 500,000 Daltons. Membranes with a MWCO greater than approximately 500,000 Daltons are preferred over about 500,000 Dalton MWCO membranes because about 500,000 Dalton MWCO membranes tend to permit transfer of a significantly smaller amount of κ-casein macropeptide, such as glycomacropeptide, into the microfiltration permeate 46, as compared to the amount of κ-casein macropeptide, such as glycomacropeptide, allowed into the microfiltration permeate 46 by membranes with an MWCO greater than about 500,000 Daltons.

As stated above, when the approximately 1,000,000 Dalton MWCO membrane is used to filter a clear aqueous solution containing only molecules with a molecular weight of approximately 1,000,000 Daltons, 95 weight percent of the molecules will be retained by the membrane, while 5 weight percent of the molecules will pass through the membrane. Though the approximate 1,000,000 Dalton MWCO membrane may also be classified in terms of a nominal pore size, this nominal pore size designation is not necessarily an accurate determinant of the range of size or particles that will pass through the membrane. This inaccuracy arises because the nominal pore size designation does not take into account the range and distribution of membrane pore sizes. For this reason, membranes that each have a particular nominal pore size will not necessarily have the same molecular weight cut-off or the same filtration characteristics for a particular feed material.

Since membranes made of ceramic, PVDF, and polysulfone will each have different pore size ranges and distributions, the range and distribution of particle sizes that permeate through approximately 1,000,000 Dalton MWCO membranes that each have a nominal pore diameter in the range of about 0.02 microns to less than about 0.2 microns will typically differ, depending upon the material the membrane is made of. Therefore, when seeking particular permeate characteristics, such as particles having a particular range and distribution of sizes, it is important to specify not only the MWCO of the membrane, but also the material the membrane is made of. Thus, as noted above, the preferred microfiltration membrane for the microfilter 42 has a MWCO greater than approximately 500,000 Daltons and is made of PVDF.

The temperature of the feed to and in the microfilter 42 may generally range from about 40° F. to less than about 140° F., with a temperature of about 70° F. being preferred. The particular temperature selected depends on a number of different competing variables, such as fat retention, polymerized protein retention, and glycomacropeptide passage along with the amount of bacterial growth inhibition. Similar comments apply when diafiltration is practiced using the microfilter 42.

The filtration characteristics of the microfiltration membrane employed in the microfilter 42 are also pressure dependent. Any pressure may be maintained on the feed (cooled protein solution 40) to the microfilter 42 proximate the microfiltration membrane, and any pressure may be maintained on the microfiltration retentate 44 (or optionally the diafiltered form of the retentate 44) downstream of the microfiltration membrane, so long as sufficient trans-membrane pressure drop exists to accomplish filtration. For example, the pressure on the feed to the microfiltration membrane may generally range from about 1 psig up to about 30 psig, while the pressure maintained on the microfiltration retentate 44 (or optionally the diafiltered form of the retentate 44) from the microfiltration membrane may range from slightly under the feed pressure down to about 0 psig. Lower pressures on the feed to the microfiltration membrane on the order of about 20 psig or less are preferred to minimize the cost of building and maintaining the system that incorporates the microfiltration membrane.

Furthermore, it has been determined that lower trans-membrane pressure drops are preferred. For example, when the pressure on the microfiltration retentate 44 downstream of the microfiltration membrane is maintained at about 4.5 psig and the pressure on the microfiltration permeate 46 from the microfilter 42 is maintained at about 3 psig, the pressure on the feed (cooled protein solution 40) proximate the microfiltration membrane is preferably about 18 psig or less. As used herein, trans-membrane pressure drop is equal to:

((P_{Feed}+P_{Retentate})/2) P_{Penneate}

where P_{Feed} is the pressure on the feed proximate the microfiltration membrane, P_{Retentate} is the pressure on the microfiltration retentate 44 downstream of the microfiltration membrane, and P_{Permeate} is the pressure on the microfiltration permeate 46 from the microfilter 42. Lower trans-membrane pressure drops are preferred because it has been found that transmission of κ-casein macropeptide, such as glycomacropeptide, through the microfiltration membrane, along with retention of the polymerized protein on the microfiltration membrane, are generally enhanced at lower trans-membrane pressure drops of about 15 psig. It is believed that increases in the trans-membrane pressure drop above about 15 psig facilitate the formation of a secondary membrane proximate the microfiltration membrane.

Secondary membrane effects arise when aggregated particles of retained molecules form what is commonly referred to as a “secondary membrane” proximate the surface of the microfiltration membrane. This “secondary membrane” tends to act as a secondary control, beyond the primary control of the membrane pore size distribution, on the size of the particles that can pass through the microfiltration membrane. The secondary membrane effect is most pronounced when the flow of solution to be filtered is directed approximately perpendicular to the surface of the microfiltration membrane. Secondary membrane effects are substantially diminished when the flow of the solution to be filtered is directed tangentially along the filtration membrane surface so that any tendency for particles to build up proximate the filtration membrane is predominantly negated by a scrubbing or sweeping action of the tangentially-directed solution. Any secondary membrane that forms tends to limit the percentage of native and soluble protein that permeates through the microfiltration membrane by preventing the native and soluble protein from reaching the membrane.

The membrane structure of the microfiltration membrane for the microfilter 42 may have a variety of configurations and designs. For example, the microfiltration membrane of the microfilter 42 maybe planar in structure or spiral-wound in structure. Preferably, the microfiltration membrane is spiral-wound in structure, versus being planar in structure, because the spiral-wound structure permits placement of a larger amount of microfiltration membrane surface in a given volumetric space than is possible with the planar construction.

Spiral-wound membranes may include any number of spiral layers ranging from two or three up to seventeen or eighteen or more. When the microfiltration membrane is spiral-wound in structure, the membrane may be encased in a plastic mesh tube for convenience of handling and to keep the spiral-wound layers in place. The spiral-wound membrane and the encasing plastic mesh tube are inserted into a cylindrical metal cannister having an inlet for the feed and outlets for the permeate and retentate, prior to use. In relation to placement of the spiral-wound membrane in the cannister, all necessary precautions should be taken to minimize, and preferably eliminate, the possibility that any feed may bypass the spiral-wound membrane and thereby flow directly to either the permeate outlet or the retentate outlet of the microfilter without being acted upon by the spiral-wound microfiltration membrane.

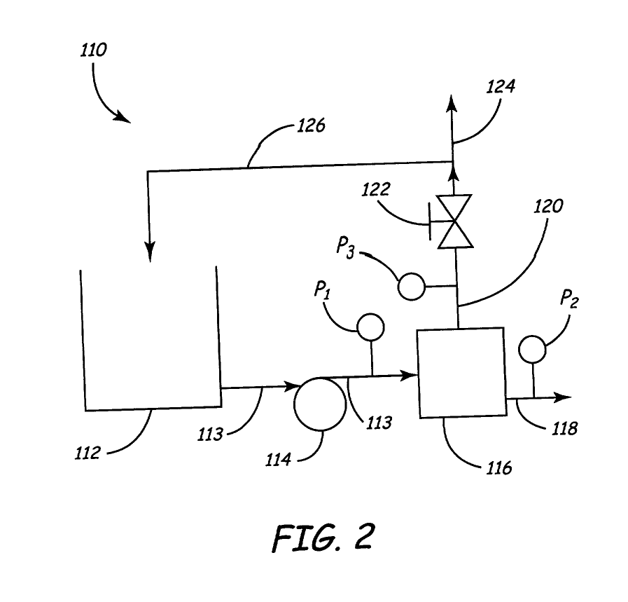

The microfilter 42 and the ultrafilters 20 and 52 may each be operated in either batch-wise fashion or in continuous mode. An exemplary batch-wise filtration unit that is suitable for use as any of the microfilter 42, the ultrafilter 20, and/or the ultrafilter 52 is generally depicted at 110 in FIG. 2. The filtration unit 110 may represent any one of the microfilter 42, the ultrafilter 20, and/or the ultrafilter 52. In the filtration unit 110, a tank 112 holds feed 113 that is transferred by a pump 114 at a pressure P₁ to a filtration membrane 116. The filtration membrane 116 may be a microfiltration membrane or an ultrafiltration membrane depending upon whether the filtration unit 110 is acting as the microfilter 42, the ultrafilter 20, or the ultrafilter 52. The filtration membrane 116 filters the feed 113 to create a permeate 118 and a retentate 120.

The permeate 118 may flow freely from the filtration membrane 116 or may be throttled by a valve (not shown) at a pressure P₂. Similarly, the retentate 120 may flow freely from the filtration membrane 116 or may be throttled by a valve 122 at a pressure P₃. Beyond the valve 122, the retentate 120 may be discharged as retentate 124 from the filtration unit 110 for further processing in accordance with the present invention or may be recycled as retentate 126 to the tank 112 for further concentration at the filtration membrane 116. Also, the retentate 120 may be blended with water for diafiltration using the filtration membrane 116.

Any pressures P₁, P₂, and P₃ may be maintained, so long as sufficient trans-membrane pressure drop exists across the filtration membrane 116 to accomplish filtration. For example, when the filtration membrane 116 is a microfiltration membrane, P₁ may range from about 1 psig up to about 30 psig, while P₂ and P₃ may range from slightly under P₁ down to about 0 psig. Lower P₁ pressures on the order of about 20 psig or less are preferred to minimize the cost of building and maintaining the filtration unit 110 that incorporates a microfiltration membrane as the filtration membrane 116. As another example, when the filtration membrane 116 is an ultrafiltration membrane, P¹ and P² maybe maintained within a broad range of pressures to attain acceptable ultrafiltration results, so long as the selected pressures do not cause degradation of the particular ultrafiltration membrane being used. For example, P₁ may be maintained at a pressure ranging from about 40 psig to about 120 psig, and P₃ may be maintained at a pressure ranging from about 0 psig to about 60 psig, so long as P₁P₂ is greater than 0 psig and preferably ranges from about 40 psig to about 80 psig.

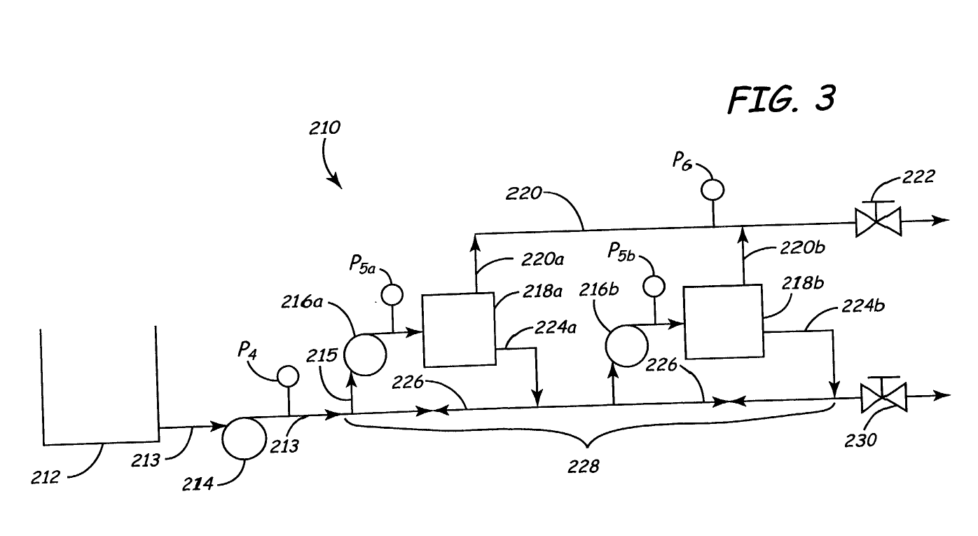

An exemplary continuous filtration unit that is suitable for use as any of the microfilter 42, the ultrafilter 20, and/or the ultrafilter 52 is generally depicted at 210 in FIG. 3. The filtration unit 210 may represent any one of the microfilter 42, the ultrafilter 20, and/or the ultrafilter 52. In the filtration unit 210, a tank 212 holds feed 213 that is transferred by a pump 214 at a pressure P₄ to a pump 216a that transfers the feed 213, as part of a feed mix 215, to a filtration membrane 218a. The filtration membrane 218a may be a microfiltration membrane or an ultrafiltration membrane depending upon whether the filtration unit 210 is acting as the microfilter 42, the ultrafilter 20, or the ultrafilter 52. The filtration membrane 218a filters the feed mix 215 to create a permeate 220a and a retentate 224a.

The permeate 220a may flow freely from the filtration membrane 218a or may be throttled by a valve 222 at a pressure 6. The retentate 224a flows into a retentate manifold 228 to form part of a retentate blend 226. When the filtration membrane 218a is the only filtration membrane in the filtration unit 210, the retentate 224a is the only retentate component of the retentate blend 226. The retentate 224a in the manifold 228 is throttled by a valve 230. The valve 230 may be set to permit discharge of none, some, or all of the retentate blend 226 from the manifold 228 and from the filtration unit 210 for further processing in accordance with the present invention. Any of the retentate blend 226 not exiting the filtration unit 210 through the valve 230 may be combined with the feed 213, prior to the pump 216a, to form the feed mix 215 that is filtered in the filtration membrane 218a.

The microfilter 42 may, in the continuous mode, employ a single filtration membrane or, alternatively, though not depicted in FIG. 1, may employ two or more individual filtration membranes arranged either in parallel or in series. Likewise, in the continuous mode, the ultrafilters 20 and/or 52 may each individually employ a single filtration unit or, alternatively, though not depicted in FIG. 1, may each individually employ two or more filtration membranes arranged either in parallel or in series. For example, the ultrafilter 20 may employ one, two, three, or even four or more separate ultrafiltration membranes arranged either in series or parallel.

As an example, the filtration unit 210 may, in addition to employing the filtration membrane 218a, employ a pump 216b that transfers retentate blend 226, pressurized to pressure P₄ by the pump 214, to a filtration membrane 218b. The filtration membrane 218b filters part of the retentate blend 226 to create a permeate 220b and a retentate 224b. The permeate 220b joins with the permeate 220a to form a permeate blend 220. As part of the permeate blend 220, the permeate 220b may flow freely from the filtration membrane 218b or may be throttled by the valve 222 at the pressure P₆.

The retentate 224b flows into the retentate manifold 228 to form part of the retentate blend 226 along with the retentate 224a. The retentate blend 226 in the manifold 228 is throttled by the valve 230. The valve 230 may be set to permit discharge of none, some, or all of the retentate blend 226 from the manifold 228 and from the filtration unit 210 for further processing in accordance with the present invention. Any of the retentate blend 226 not exiting the filtration unit 210 through the valve 230 may be transferred to the filtration membrane 218a by the pump 216a or may be transferred to the filtration membrane 218b by the pump 216b for further filtration.

Though the pump 216a is depicted as being located generally between the pump 214 and the pump 216b in the filtration unit 210, the pump 214 may alternatively be positioned between the pump 216a and the pump 216b to enhance distribution of the feed 213 between the filtration membrane 218a and the filtration membrane 218b as part of the retentate blend 226. Also, the permeate blend 220 (or the permeate 220a or the permeate 220b, individually) and/or the retentate blend 226 (or the retentate 224a or the retentate 224b, individually) may be blended with water for individual diafiltration in the filtration membrane 218a, the filtration membrane 218b, or in the filtration unit 210 that includes both the filtration membrane 218a and the filtration membrane 218b.

Any pressures P₄, P₅ₐ, P_{5b}, and P₆ may be maintained, so long as sufficient trans-membrane pressure drop exists across the filtration membranes 218a, 218b to accomplish filtration. For example, when the filtration membranes 218a, 218b are microfiltration membranes, P₅ₐ and P_{5b} may range from about 1 psig up to about 30 psig, while P₄ and P₆ may range from slightly under P₅ₐ and P_{5b} down to about 0 psig. Lower P₅ₐ and P_{5b} pressures on the order of about 20 psig or less are preferred to minimize the cost of building and maintaining the filtration unit 210 that incorporates microfiltration membranes as the filtration membranes 218a, 218b.

As another example, when the filtration membranes 218a, 218b are ultrafiltration membranes, P₁ and P₂ may be maintained within a broad range of pressures to attain acceptable ultrafiltration results, so long as the selected pressures do not cause degradation of the particular ultrafiltration membrane being used. For example, P₄ and P₆ may range from slightly under P₅ₐ and P_{5b} down to about 0 psig. Preferably, P₅ₐ and P_{5b} range from about 40 psig to about 120 psig, and P₆ is ranges from about 0 psig to about 60 psig, so long as P₅ₐ-P₆ and P_{5b}-P₆ are each greater than 0 psig and preferably each range from about 40 psig to about 80 psig.

Various analytical techniques and calculation techniques are employed herein. An explanation of these techniques and calculations follows. All determinations are on a wet basis, without drying the sample, unless otherwise specified below.

### Property Determination and Characterization Techniques

### Total Solids Determinations

To determine the weight percent total solids, wet basis, in a sample, the actual weight of total solids may be determined by analyzing the sample in accordance with Method #925.23 (33.2.09) of Official Methods of Analysis, Association of Official Analytical Chemists (AOAC) (16th Ed., 1995). The weight percent total solids, wet basis, may then be calculated by dividing the actual weight of total solids by the actual weight of the sample.

### Total Protein Determinations

To determine the percent of total protein, wet basis, in a sample, the actual weight of total protein may be determined in accordance with Method #991.20 (33.2.11) of Official Methods of Analysis, Association of Official Analytical Chemists (AOAC) (16th Ed., 1995). This method yields the actual weight of total protein in the sample. The weight percent total protein, wet basis, is calculated by dividing the actual weight of total protein by the actual weight of the sample. To determine the weight percent of total protein, dry basis, in the sample, the wet basis weight percent of total solids in the sample is determined in accordance with the previously described total solids procedure and the weight percent of total protein, wet basis, is divided by the weight percent of total solids to yield the weight percent of total protein, dry basis, in the sample.

### Whey Protein Nitrogen Determinations

The weight percent whey protein nitrogen (WPN), wet basis, in a particular sample, maybe determined in accordance with the Standards for Grades of Dry Milk, American Dry Milk Industry (1971 ed.). To determine the weight percent of whey protein nitrogen, dry basis, in the sample, the weight percent of total solids is determined in accordance with the previously described total solids procedure and the weight percent of whey protein nitrogen, wet basis, is divided by the weight percent of total solids to yield the weight percent of whey protein nitrogen, dry basis, in the sample.

### Protein Analysis Procedure No. 1

The wet basis concentrations (weight per unit volume) of glycomacropeptide, β-lactoglobulin, and α-lactalbumin that have not been denatured may be determined in a particular sample using high performance liquid chromatography (also referred to herein as “HPLC”) in accordance with this Protein Analysis Procedure No. 1. A Waters HPLC system employing a Waters M-6000A high pressure pump, a Waters 710B WISP automatic sample injection system, and a Waters 490E programmable multiwavelength detector may be used. The Waters HPLC system employing the specified components may be obtained from Waters Corporation of Milford, Mass.

The Waters HPLC system maybe used to detect β-lactoglobulin, α-lactalbumin, and glycomacropeptide that have not been denatured. For example, the Waters 490E programmable multiwavelength detector of the Waters HPLC system may be set at 280 nanometers to detect β-lactoglobulin and α-lactalbumin that have not been denatured.

As another example, the Waters 490E programmable multiwavelength detector of the Waters HPLC system may be set at 214 nanometers to detect glycomacropeptide that has not been denatured. However, prior to any glycomacropeptide analysis at the 214 nanometer detection wavelength, the sample to be analyzed should first be treated to assure that β-lactoglobulin is not present in the sample.

β-lactoglobulin must be removed from the sample prior to glycomacropeptide analysis at the 214 nanometer detection wavelength, (1) because β-lactoglobulin includes aromatic amino acid groups and is therefore, like glycomacropeptide, detected at the 214 nanometer detection wavelength and (2) because β-lactoglobulin is detected within a retention time range that overlaps the retention time range of glycomacropeptide. Therefore, absent removing β-lactoglobulin from the sample, an HPLC determination at the 214 nanometer detection wavelength for non-denatured glycomacropeptide content in the sample would likely also include non-denatured β-lactoglobulin that is present in the sample. Therefore, to make the HPLC determination at the 214 nanometer detection wavelength specific for non-denatured glycomacropeptide content, any β-lactoglobulin that would potentially interfere with the non-denatured glycomacropeptide analysis must be removed.

Removal of β-lactoglobulin from a sample to be analyzed for non-denatured glycomacropeptide content, without deleteriously affecting any non-denatured glycomacropeptide present in the sample, may be accomplished by treating the sample with trichloroacetic acid (“TCA”) to precipitate any β-lactoglobulin (and any α-lactalbumin) that are present in the sample. First, trichloroacetic acid is added to the sample in an amount that is sufficient to make the concentration of trichloroacetic acid in the sample about 8 weight percent, based upon the combined total weight of the sample and the added trichloroacetic acid, which is sufficient to precipitate any β-lactoglobulin (and any α-lactalbumin) present in the sample. The treated sample may then be centrifuged to remove precipitated β-lactoglobulin (and precipitated α-lactalbumin).

After removal of precipitated β-lactoglobulin (and precipitated α-lactalbumin), the sample may thereafter be analyzed in the Waters HPLC system with the Waters 490E programmable multiwavelength detector set at 214 nanometers to detect glycomacropeptide that has not been denatured. This glycomacropeptide analysis procedure that incorporates the initial TCA treatment step is similar to the glycomacropeptide analysis procedure that is described in B. Lieske and G. Conrad, “A New Method to Estimate Caseinomacropeptide and Glycomacropeptide From Trichloroacetic Acid Filtrates,” Milchwissenschaft, Vol. 51, pp 431-435, (1996), that is incorporated by reference herein, in its entirety.

In the Waters HPLC system, as an optional approach, the Waters 490E programmable multiwavelength detector maybe used to simultaneously (1) detect any non-denatured (native) β-lactoglobulin and any non-denatured (native) α-lactalbumin in the sample at a detection wavelength of 280 nanometers, and (2) detect any non-denatured (native) glycomacropeptide in the same sample at a detection wavelength of 214 nanometers. This simultaneous detection ability on the same sample at detection wavelengths of 214 nanometers and 280 nanometers is useful when measuring native glycomacropeptide content in the sample, since it allows native glycomacropeptide content measured, and documented at a particular retention time range, using the 214 nanometer detection wavelength, while simultaneously confirming that no native β-lactoglobulin peaks appear within the retention time range (where native glycomacropeptide peaks appear at the 214 nanometer detection wavelength), when using the 280 nanometer detection wavelength that is employed for the native β-lactoglobulin.

This ability to confirm that β-lactoglobulin is not present in the sample being analyzed for non-denatured glycomacropeptide is important, since, as explained above, any β-lactoglobulin present in the sample being analyzed for non-denatured glycomacropeptide at the 280 nanometer detection wavelength would potentially interfere with the non-denatured glycomacropeptide. Therefore, by running the same sample simultaneously at the glycomacropeptide determination wavelength (280 nanometers) and at another wavelength (214 nanometers) where β-lactoglobulin is detected, one may confirm that β-lactoglobulin, which could interfere with the non-denatured glycomacropeptide determination at the 280 nanometer detection wavelength, is not present in the sample and consequently will not interfere with the non-denatured glycomacropeptide determination for the sample under consideration.

In the Waters High Performance Liquid Chromatography system, during analysis for glycomacropeptide, β-lactoglobulin, and/or α-lactalbumin that have not been denatured, the stationary phase of the chromatographic system may be a 300 mm×7.8 mm Bio-Sil SEC 125 size exclusion column that is obtained from Bio-Rad Corp. of Hercules, Calif. The mobile phase of the chromatographic system may be a solution of 0.1M sodium sulfate and 0.1M sodium phosphate with a pH of 6.0. Volumetric standards for glycomacropeptide, β-lactoglobulin and α-lactalbumin that have not been denatured may be obtained from Sigma Chemical Company of St. Louis, Mo. The sample flow rate in the system may be set at 1.0 ml/minute.

Peak area data may be collected using the EZ Chrom ChromatographyData System that is available from Scientific Software, Inc. of San Ramon, Calif. and maybe analyzed using the SuperCompare feature of the EZ Chrom ChromatographyData System. Using the peak area data for the sample and the volumetric standards for glycomacropeptide, β-lactoglobulin, and α-lactalbumin that have not been denatured, the EZ Chrom Chromatography Data System will calculate the volumetric concentrations of non-denatured glycomacropeptide, non-denatured β-lactoglobulin, and non-denatured α-lactalbumin in the sample.

### Protein Analysis Procedure No. 2

The wet basis concentrations (weight per unit volume) of κ-casein macropeptide, α-lactalbumin, and β-lactoglobulin that have not been denatured may be determined in a particular sample using Reversed Phase HPLC in accordance with this Protein Analysis Procedure No. 2.

In this Protein Analysis Procedure No. 2, a Waters LC Module 1 HPLC system employing a Waters M-6000A high pressure pump, a Waters 710B WISP automatic sample injection system, and a Waters 431 variable wavelength detector may be used. The Waters LC Module 1 HPLC system employing the specified components may be obtained from Waters Corporation of Milford, Mass.

The Waters 431 variable wavelength detector of the Waters LC Module 1 HPLC system may be set at 214 nanometers to detect κ-casein macropeptide that has not been denatured. This κ-casein macropeptide determination procedure is described in more detail in D. F. Elgar, C. S. Norris, J. S. Ayers, M. Pritchard, D. E. Otter, and K. P. Palmano, Journal of Chromatography A, Vol. 878, pp 183-196, (2000) that is incorporated by reference herein, in its entirety.

In this Protein Analysis Procedure No. 2, the Waters LC Module 1 HPLC system may employ a 3-ml Resource RPC column as the stationary phase. The 3-ml Resource RPC column may be obtained from Amersham Pharmacia Biotech of Piscataway, N.J. In this Protein Analysts Procedure No. 2, the Waters LC Module 1 HPLC system may employ a pair of mobile phase solvents, namely Solvent A and Solvent B. Solvent A may be a solution of trifluoroacetic acid in purified water with a concentration of 0.1 volume percent trifluoroacetic acid, based upon the total volume of Solvent A. Solvent B may be a solution of trifluoroacetic acid in 90% acetonitrile in purified water with a concentration of 0.9 volume percent trifluoroacetic acid, based upon the total volume of Solvent B, and with a concentration of 90 volume percent acetonitrile, based upon the total volume of Solvent. The purified water used in Solvent A and in Solvent B may be prepared using a Barnstead NANOpure Infinity water system that may be obtained from Barnstead/Thermolyne Corporation of Dubuque, Iowa. The purified water used in Solvent A and in Solvent B had a resistance of about 18 Mega-ohms (about 0.06 μS/cm), unless otherwise stated herein.

The 3-ml Resource RPC column that is used as the stationary phase may be calibrated with a mobile phase mixture containing 80 weight percent Solvent A and 20 weight percent Solvent B, based upon the total weight of the mobile phase mixture. The solvent gradient using this mobile phase mixture is shown in Table 1 below:

TABLE 1

| TIME | FLOW | WEIGHT % | WEIGHT % | |
|---|---|---|---|---|
| (seconds | (ml/min) | SOLVENT A | SOLVENT B | CURVE |
| INITIAL | 1.00 | 80 | 20 | |
| 3 | 1.00 | 80 | 20 | 6 |
| 21 | 1.00 | 60 | 40 | 6 |
| 51 | 1.00 | 55 | 45 | 6 |
| 90 | 1.00 | 50 | 50 | 6 |
| 93 | 1.00 | 50 | 50 | 6 |
| 102 | 1.00 | 30 | 70 | 6 |
| 105 | 1.00 | 0 | 100 | 6 |
| 108 | 1.00 | 0 | 100 | 6 |
| 114 | 1.00 | 80 | 20 | 6 |
| 135 | 1.00 | 80 | 20 | 6 |
| 140 | 0 | 80 | 20 | 6 |

The sample flow rate in the Waters LC Module 1 HPLC system may be set at 1.0 ml/minute. Peak area data may be collected using the EZ Chrom Chromatography Data System that is available from Scientific Software, Inc. of San Ramon, Calif. and may be analyzed using the SuperCompare feature of the EZ Chrom Chromatography Data System.

As noted above, this Protein Analysis Procedure No. 2 employs reversed phase HPLC, as opposed to the more traditional HPLC of Protein Analysis Procedure No. 1. Different macropeptides, such as different κ-casein macropeptides, exhibit a range of different polarities. Some macropeptides, including some types of κ-casein macropeptides, such as glycosylated macropeptides, are quite polar. Other macropeptides, such as non-glycosylated macropeptides, are less polar than glycosylated macropeptides. Furthermore, there are other macropeptides, including some types of κ-casein macropeptides, that are less polar than non-glycosylated macropeptide.

For a sample containing non-glycosylated macropeptide and macropeptides less polar than non-glycosylated macropeptide, the more traditional HPLC of Protein Analysis Procedure No. 1 fails to detect, or detects only a very small amount of, the non-glycosylated macropeptide and macropeptides less polar than non-glycosylated macropeptide. This failure is due to use of TCA in Protein Analysis Procedure No. 1, which causes much, if not all, of the non-glycosylated macropeptide and macropeptides less polar than non-glycosylated macropeptide to be precipitated out of the sample, prior to the HPLC analysis.

On the other hand, the reversed-phase HPLC technique of Protein Analysis Procedure No. 2 does not employ TCA or any type of TCA precipitation. In the reversed-phase HPLC technique of Protein Analysis Procedure No. 2, the retention time for β-lactoglobulin is different from the retention time for κ-casein macropeptides, since the elution system for the reversed-phase HPLC technique of Protein Analysis Procedure No. 2 depends more on polarity than on molecular size. Therefore, in the reversed-phase HPLC technique of Protein Analysis Procedure No. 2, β-lactoglobulin does not mask or interfere with accurate macropeptide determinations and TCA is not needed. Since TCA is not employed in the reversed-phase HPLC technique of Protein Analysis Procedure No. 2, the full complement of macropeptides, including the full complement of κ-casein macropeptides, remains in the sample for detection.

When a sample containing macropeptides, such as κ-casein macropeptides, is analyzed using the reversed-phase HPLC technique of Protein Analysis Procedure No. 2, the most polar macropeptides, such as any macropeptides that are glycosylated and/or phosphorylated, are detected (taken off the chromatographic column) first. Then, less polar macropeptides, such as macropeptides that are not glycosylated or phosphorylated, are detected, and the least polar macropeptides, are detected last. Consequently, in a detection scan from the reversed-phase HPLC technique of Protein Analysis Procedure No. 2, the more polar macropeptides with the longest retention time, such as glycomacropeptides, appear toward the left of the scan, and less polar macropeptides with shorter retention times, moving from left to right in the scan by decreasing polarity, follow the more polar macropeptides.

Ultimately, Protein Analysis Procedure No. 2 differs from Protein Analysis Procedure No. 1 above in that Protein Analysis Procedure No. 2 detects all of the non-denatured κ-casein macropeptide present in the sample, whereas Protein Analysis Procedure No. 1 only detects non-denatured, glycosylated forms of κ-casein macropeptide (i.e.: glycomacropeptide). Additionally, Protein Analysis Procedure No. 2 differs from Protein Analysis Procedure No. 1 above in that Protein Analysis Procedure No. 2, at a detection wavelength of 214 nanometers, also detects any α-lactalbumin and any β-lactoglobulin (actually two different forms of β-lactoglobulin: β-Lg A and β-Lg B) present in the sample, whereas Protein Analysis Procedure No. 1, at a detection wavelength of 214 nanometers, does not detect any α-lactalbumin or any β-lactoglobulin that is present in the sample, since the TCA used in Protein Analysis Procedure No. 1 precipitates all α-lactalbumin and/or β-lactoglobulin from the sample prior to HPLC analysis of the sample.

### Lactose Determinations

To determine the weight percent lactose, wet basis, in a sample, the actual weight of lactose in the sample may be determined using analysis kit number 176-303, that is available from Boehringer-Mannheim of Indianapolis, Ind. in accordance with the procedural instructions included with analysis kit number 176-303. The weight percent lactose, wet basis, may then be calculated by dividing the actual weight of lactose in the sample by the actual weight of the sample. To determine the weight percent of lactose, dry basis, in the sample, the weight percent of lactose in the sample is determined in accordance with the previously described total solids procedure and the weight percent of lactose, wet basis, is divided by the weight percent of total solids to yield the weight percent of lactose, dry basis, in the sample.

### Fat Determinations

To determine the weight percent fat, wet basis, in a sample, the actual weight of fat in the sample may be determined in accordance with Method #974.09 (33.7.18) of Official Methods of Analysis, Association of Official Analytical Chemists (AOAC) (16th Ed., 1995). The weight percent fat, wet basis, may then be calculated by dividing the actual weight of fat in the sample by the actual weight of the sample. To determine the weight percent of fat, dry basis, in the sample, the weight percent of fat in the sample is determined in accordance with the previously described total solids procedure and the weight percent of fat, wet basis, is divided by the weight percent of total solids to yield the weight percent of fat, dry basis, in the sample.

### Viscosity Determinations

Unless otherwise indicated, all viscosities recited herein maybe determined in accordance with the following procedure that uses a Brookfield Model DV-II+ Digital Viscometer. Viscosity determinations may be made using the Brookfield Model DV-II+ Digital viscometer in accordance with Operating Instructions Manual M/92-1616-C-892 supplied by Brookfield Engineering Laboratories.

The Brookfield viscosity of a particular sample may generally be determined by placing an appropriate amount of the sample in a beaker that is positioned within the viscosity measurement cell of the viscometer. The beaker should have a diameter that is sufficient to prevent development of any wall effects that could affect, at least by more than a de minimis degree, the viscosities determined by this procedure. The temperature of the sample may be taken immediately after placement of the sample in the beaker and subsequently recorded. The viscosity determination should be made quickly to minimize, and preferably eliminate, or essentially eliminate, any change in the temperature of the sample following placement of the sample in the beaker. A variety of different disks and T-Bars may be used as the spindle during viscosity determination.

T-Bars are sometimes employed when determining the viscosity of non-flowing or slow-flowing sample materials, such as pastes, crams, and gels present problems. Conventional disks, when employed as the rotating spindle during viscosity determination, tend to push the sample material aside, which results in a continuously decreasing viscosity reading. A solution to that problem is a special stand which slowly raises and lowers the viscometer while a special T-bar spindle rotates in the sample material. The crossbar of the spindle thus continuously cuts into fresh material and describing a helical path through the sample as the spindle rotates.

The spindle is positioned in the sample within the beaker immediately after the sample has been placed in the beaker. The spindle, identified by a spindle designation, is selected so the measured Brookfield viscosity is within the measurement range of the spindle. As noted, a variety of different disks and T-Bars may be used as the spindle during viscosity determinations. Viscosity determinations that are described in this document variously used an “A” T-bar, a “B” T-bar, “Disk 1”, “Disk 3,” “Disk 6,” and “Disk 7” as the spindle and as described elsewhere in this document. The “A” T-bar, The “B” T-bar, “Disk 1”, “Disk 3,”. “Disk 6,” and “Disk 7” that were used as spindles are each available from Brookfield Engineering Laboratories. During viscosity determinations made herein in accordance with this viscosity determination procedure, the selected spindle was rotated at a rate of about twenty (20) revolutions per minute during viscosity determinations, unless stated otherwise.

### EXAMPLES

The present invention is more particularly described in the following examples which are intended as illustrations only since numerous modifications and variations within the scope of the present invention will be apparent to those skilled in the art.

### Example 1

This Example demonstrates processing of a starting dairy material that contains glycomacropeptide, α-lactalbumin, and β-lactoglobulin to produce a polymerized protein powder that is rich in polymers of α-lactalbumin and the β-lactoglobulin and a GMP powder that is rich in glycomacropeptide. In this Example, sweet cheese whey derived from the manufacture of a cheddar cheese was processed to form a whey protein isolate containing approximately 27 weight percent total protein, based upon the total weight of the whey protein isolate. The whey protein isolate was immediately cooled to about 40° F. after production and was placed in 240 gallon portable tanks. The portable tanks were held at about 40° F. prior to further processing of the whey protein isolate. The pH of the whey protein isolate was in the range from about 6.2 to about 6.4 standard pH units, which indicates that good handling procedures were observed.

Subsequent aspects of this Example were conducted in accordance with the schematic drawing of FIG. 1, where the whey protein isolate is substituted in place of the ultrafiltration retentate 22. Fourteen gallons of the whey protein isolate (which served as the ultrafiltration retentate 22 in this Example) were diluted with 86 gallons of reverse osmosis water (dilution water 26) to produce the diluted intermediate 28 with a concentration of about 3.8 weight percent total protein, based upon the total weight of the diluted intermediate 28. The total volume of the diluted intermediate 28 was about 100 gallons.

Ten weight percent sodium hydroxide, as the alkaline agent 30, was added to the diluted intermediate 28 to yield the alkaline intermediate 32 with a pH of about 8.0. While stirring the alkaline intermediate 32 gently in a vat, the intermediate 32 was heated to a temperature of about 190° F. by injecting culinary grade steam 34 into the intermediate 32. This heating of the intermediate 32 transformed the alkaline intermediate 32 into the heated intermediate 36 within the vat. The intermediate 36 included about 15 gallons of additional water that were not present in the intermediate 32, due to condensation of the culinary grade steam 34 that was injected into the intermediate 32. The intermediate 36 was held at 190° F. for about 10 minutes and was thereafter cooled to a temperature of about 120° F. to form the cooled protein solution 40. The cooling of the intermediate 36 was accomplished using a cooling water jacket that surrounded the vat containing the intermediate 36.

The cooled protein solution 40 was then microfiltered in the microfilter 42, without adjusting the pH of the solution 40. The microfilter 42 consisted of an AF-series microfiltration membrane structure obtained from PTI Advanced Filtration, Inc. of San Diego, Calif. that was positioned in a microfiltration canister. The membrane of the microfiltration membrane structure was manufactured of polyvinyl difluoride (PVDF) with a molecular weight cut-off (MWCO) of about 500,000 Daltons and a nominal pore diameter in the range of about 0.02 microns to less than about 0.2 microns. The AF-series membrane structure included about 17-18 spirals of the membrane sheet. The distance between facing spirals of the membrane sheet was about 65 mils.

The cooled protein solution 40 was concentrated to minimum volume via batch filtration, flowed by batch diafiltration, in the microfilter 42. As a result of the batch filtration in the microfilter 42, the volume of the microfiltration retentate 44 in this Example was concentrated about 5 times versus the volume of the cooled protein solution 40 that was used as feed to the microfilter 42. After processing of the cooled protein solution 40, the microfiltration retentate 44 was then batch diafiltered in the microfilter 42 using reverse osmosis water with a volume approximately double that of the microfiltration retentate 44.

The microfilter 42, during both the initial filtration and the subsequent diafiltration, was operated to maintain less than about 18 psig inlet pressure to the membrane structure and the back pressure on the microfiltration retentate 44 was about 3 psig. The temperature of the cooled protein solution 40 during the microfiltration and the temperature of the feed during the subsequent diafiltration were each maintained at about 120° F. The diafiltered microfiltration retentate 44 was collected and saved for use in subsequent portions of this Example. The microfiltration permeate 46 that was obtained during the initial microfiltration of the cooled protein solution 40 was combined with the microfiltration permeate 46 obtained during diafiltration of the microfiltration retentate 44. This combined microfiltration permeate 46 stream was also saved for future use in this Example.

The diafiltered microfiltration retentate 44 was divided into two equal portions that are subsequently identified herein as a first retentate 44 portion and a second retentate 44 portion. The pH of the first retentate portion 44 was adjusted to about 6.3 by addition of 10 weight percent phosphoric acid with stirring. The phosphoric acid was added very slowly and a precipitate formed at the site of the addition. However, this precipitate readily redissolved upon additional stirring of the now acidified first retentate 44 portion. Both the acidified first retentate 44 portion and the second retentate 44 portion were spray dried to form a first spray dried powder and a second spray dried powder, respectively.

Details about the volumes, component concentrations, and component weights for the whey protein isolate (which served as the ultrafiltration retentate 22 in this Example); the intermediates 28, 32, and 36; the acidified first retentate 44 portion and the second retentate 44 portion are provided in Table 2 below:

TABLE 2

| | | Total | Total | Total | Total |
|---|---|---|---|---|---|
| | Volume | Solids | Protein | Solids | Protein |
| Sample Description | (gal) | (wt %) | (wt %) | (lbs.) | (lbs.) |
| Whey Protein Isolate | 100 | 3.84 | 3.41 | 33.0 | 29.3 |
| (as UF 1 retentate 22) | | | | | |
| Alkaline (pH ˜8) | 100 | 3.86 | 3.38 | 33.2 | 29.1 |
| Intermediate 32 | | | | | |
| Heated Intermediate 36 | 115 | 3.42 | 3.00 | 33.8 | 29.7 |
| Second Retentate 44 | 10 | 6.29 | 5.95 | 5.4 | 5.1 |
| Portion (pH ˜8) | | | | | |
| Acidified First Retentate | 10 | 6.28 | 5.93 | 5.4 | 5.1 |
| 44 Portion (@ pH ˜6.3) | | | | | |

From Table 2, it is apparent that about one third of the total protein originally present in the feed material (whey protein isolate) remained with the microfiltration retentate 44 (no matter whether considering the acidified first retentate 44 portion or the second retentate 44 portion), whereas about two thirds of the total protein content of the feed material (whey protein isolate) passed into the microfiltration permeate 46.

Also, component concentration details and mineral weight details for the first spray dried powder and for the second spray dried powder are provided below in Table 3:

TABLE 3

| | First Spray | Second Spray |
|---|---|---|
| | Dried Powder | Dried Powder |
| | (@ pH ˜6.3)* | (@ pH ˜8)** |
| Moisture (wt %) | 5.12 | 6.41 |
| Protein (wt %) | 91.53 | 90.74 |
| Fat (wt %) | 0.19 | 0.18 |
| Ash (wt %) | 2.36 | 2.28 |
| CHO by diff (wt %) | 0.80 | 0.39 |
| Sodium (mg) | 283 | 225 |
| Potassium (mg) | 274 | 268 |
| Calcium (mg) | 531 | 400 |
| Phosphorus (mg) | 494 | 31 |
| Chloride (wt %) | 0.09 | 0.09 |

*Spray Dried Form of Acidified First Retentate 44 Portion (acidified to a pH of ˜6.3)
**Spray Dried Form of Second Retentate 44 Portion that had a pH of ˜8

The results of Table 3 demonstrate that both the first spray dried powder and the second spray dried powder have protein concentrations on the order of about 96 to 97 weight percent, on a dry basis. This high protein content indicates that carbohydrates associated with glycomacropeptide are removed as part of the microfiltration permeate 46 and are predominantly non-existent in the two spray dried powders.

Twenty grams of the first spray dried powder were dissolved in 180 grams of reverse osmosis water that had a temperature of about 72° F. to make a 10 weight percent solution. Also, twenty grams of the second spray dried powder were dissolved in 180 grams of reverse osmosis water to make a 10 weight percent solution. Both solutions were stirred with the aid of a magnetic stirrer to help disperse the powder in the water. Both the first spray dried powder and the second spray dried powder dissolved in the water with considerable foaming. It was observed that none of the polymerized protein powder precipitated in either of the 10 weight percent solutions within a period of about six hours after preparation of the 10 weight percent solutions.

Next, a solution of the whey protein isolate (which served as the ultrafiltration retentate 22 in this Example) was dissolved in reverse osmosis water to provide a solution containing 10 weight percent whey protein isolate, based upon the total weight of the whey protein isolate solution. Then, comparisons of the time required for the same volumes of (1) water, (2) the whey protein isolate solution, (3) the solution containing the first spray dried powder, and (4) the solution containing the second spray dried powder to flow from a 25 milliliter pipet were conducted. The results of this test are provided in Table 4 below:

TABLE 4

| | Empyting |
|---|---|
| Sample Description | time (sec) |
| Water | 34 |
| 10 wt. % Production WPI in water | 45 |
| 10 wt. % First Spray Dried Powder (@ pH 6.3)* in water | 90 |
| 10 wt. % Second Spray Dried Powder (@ pH 8)** in water | 150 |

*Spray Dried Form of Acidified First Retentate 44 Portion (acidified to a pH of ˜6.3)
**Spray Dried Form of Second Retentate 44 Portion that had a pH of ˜8

These results of Table 4 demonstrate that the viscosity of the solution of the second spray dried powder cause the solution of the solution of the second spray dried powder to drain significantly more slowly from the pipette than the solution of the first spray dried powder. This demonstrates that the solution of the second spray dried powder had a significantly higher viscosity than the solution of the first spray dried powder. Likewise, the solution of the first spray dried powder took significantly longer to drain from the pipet than the solution of the whey protein isolate. This demonstrates that the solution of the first spray dried powder had a significantly higher viscosity than the solution of the whey protein isolate.

For purposes of quantifying these viscosity results, viscosity determinations were then quantified using the Viscosity Determination procedure set forth in the Property Determination and Characterization Techniques section of this document. Viscosity determinations were made on both the solution of the first spray dried powder and on the solution of the second spray dried powder that are described above. A viscosity determination was also made on the 10% solution of the whey protein isolate that is described above.

All samples were at room temperature (about 72° F.) during the viscosity determinations made with the Brookfield viscometer. Viscosity determinations for solution of the first spray dried powder and for the solution of the second spray dried powder using both a disk and then a T-Bar as the spindle, while the viscosity determination for the solution of the whey protein isolate was made only with a disk as the spindle. During all viscosity determinations of this Example that employed the Brookfield viscometer, the selected spindle was rotated at a rate of about twenty (20) revolutions per minute during each viscosity determination. The results of the viscosity determinations using the Brookfield viscometer, with the Brookfield viscosities shown in centipoise (cp), are provided in Table 5 below:

TABLE 5

| Sample Description | Spindle | Viscosity (cp) |
|---|---|---|
| 10 wt. % Production WPI in water | Disk 1 | ≈0.5 |
| 10 wt. % First Spray Dried Powder | Disk 6 | 8,300 |
| (@ pH 6.3)* in water | | |
| 10 wt. % First Spray Dried Powder | T-Bar A | 8,000 |
| (@ pH 6.3)* in water | | |
| 10 wt. % Second Spray Dried Powder | Disk 7 | 12,000 |
| (@ pH 8)** in water | | |
| 10 wt. % Second Spray Dried Powder | T-Bar B | 11,000 |
| (@ pH 8)** in water | | |

*Spray Dried Form of Acidified First Retentate 44 Portion (acidified to a pH of ˜6.3)
**Spray Dried Form of Second Retentate 44 Portion that had a pH of ˜8

These results of Table 5 again demonstrate that the solution of the second spray dried powder had a significantly higher viscosity than the solution of the first spray dried powder. Likewise, these results demonstrate that both the solution of the first spray dried powder and the solution of the second spray dried powder had a significantly higher viscosity than the solution of the whey protein isolate.

Thus, the heat treatment conducted on the intermediate 36 resulted in polymerization of protein that allowed the resulting first spray dried powder and second spray dried powder to form solutions with enhanced viscosity, as compared to the viscosity of a comparable solution of the whey protein isolate of this Example that had not been heat treated in accordance with the present invention. Furthermore, leaving the second retentate 44 portion of this Example with its original alkaline pH caused the second spray dried powder to form the solution with significantly more viscosity than the solution formed by the first spray dried powder that was based upon the acidified first retentate 44 portion. Thus, depending upon the desired viscosity characteristics, the microfiltration retentate 44 that contains polymerized protein may be either left with an alkaline pH or with an acidic pH, depending upon the particular water absorbency and viscosity properties required for the polymerized protein.

The solutions of both the first spray dried powder and the second spray dried powder were separately boiled and placed into containers that were refrigerated overnight. Both of the refrigerated mixtures formed clear gels that could be dug out of the containers and handled manually in gel form. The gels that were formed based upon the solutions of the first spray dried powder and the second spray dried powder were both quite porous and contained entrained bubbles that interfered with determination of gel strength using either the axial method or the torsional method. The microscopic observation of both of the produced gels showed that each of the gels had a granular structure.

Solutions containing different concentrations of the first spray dried powder and the second spray dried powder were examined using the least concentration end point (“LCE”) gelation test. A minimum protein concentration is necessary for gelation. Low protein concentrations disfavor intramolecular protein interactions that form a gel structure. The minimum protein concentration that is required to form a gel is the basis of the LCE test procedure that has been employed by scientists to provide a useful comparison on the gelling properties of different protein solutions as a function of pH, ion content, and other compositional and processing variables. The LCE test may be conducted in accordance with the procedure set forth in Brandenberg, A. H., Morr, C.V., and Weller, C. L., “Gelation of Commercial Whey Protein Concentrates: Effect of Removal of Low-Molecular Weight Components,” Journal of Food Science, Vol. 57, pp 427-432 (1992).

The LCE gelation test showed that the gel strength of gelable solution that were based on the first spray dried powder were consistently stronger than the gel strengths of gelable solutions that were based upon the second spray dried protein powder. Also, both with and without salt addition, the first spray dried powder had a water holding capacity of 100 percent, whereas the second spray dried powder had a water holding capacity of 75 percent without addition of salt, but a water holding capacity of 100 percent with the addition of salt.

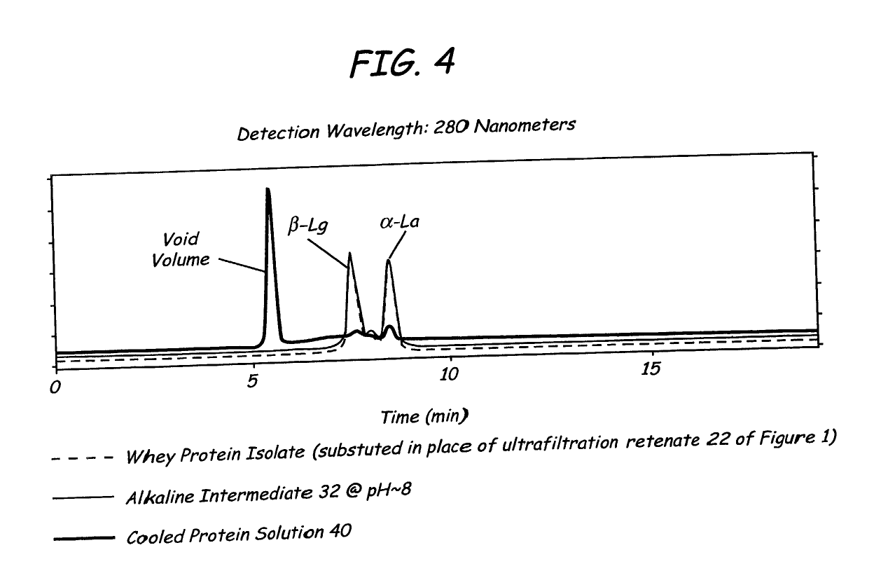

Samples of various streams described above in this Example were subjected to HPLC analysis based upon size-exclusion in accordance with Protein Analysis Procedure No. 1 that is provided above in the Property Determination And Characterization Techniques section of this document. In FIG. 4, the concentrations of both α-lactalbumin and β-lactoglobulin are essentially the same, if not the same, in both the feed material (whey protein isolate) used in this Example and in the alkaline intermediate 32. On the other hand, after heating, FIG. 4 demonstrates that very little α-lactalbumin or β-lactoglobulin remains in the cooled protein solution 40.

In FIG. 4, the void volume peak is very large for the cooled protein solution 40, while the α-lactalbumin and the β-lactoglobulin peaks are small for the cooled protein solution 40. In size exclusion HPLC, polymers that are insoluble in the solution (here, an aqueous solution) being analyzed are excluded from the column by filtration frits. Also, in size exclusion HPLC, larger molecules that are soluble in the solution being analyzed and therefore enter the column are less likely to enter the pores of the absorbent gel and are therefore eluted first in the column. The large void volume peak of the cooled protein solution 40 sample represents relatively large molecules, namely polymers of α-lactalbumin and β-lactoglobulin that are formed by sulfur bridging between cysteine residues of the original α-lactalbumin and β-lactoglobulin. The fact that the polymers of α-lactalbumin and β-lactoglobulin represented by the large void volume peak in FIG. 4 are observed in the scan at all indicates that these polymers of α-lactalbumin and β-lactoglobulin, by virtue of entering the HPLC column, are soluble in the aqueous solution being analyzed by the HPLC procedure.

The position of the large void volume peak in the FIG. 4 trace reflects the size of the polymers of α-lactalbumin and β-lactoglobulin in the cooled protein solution 40. If the position of the large void volume peak were at the very front (leftmost position) of the trace, this would indicate that the polymers of α-lactalbumin and β-lactoglobulin in the cooled protein solution 40 have a molecular weight greater than about 300,000 Daltons.

Instead, the position of the large void volume peak in FIG. 4 appears somewhat to the right of the front of the trace. Indeed, the position of the large void volume peak, relative to the positions of the peaks for α-lactalbumin and β-lactoglobulin, respectively, indicates that the polymers of α-lactalbumin and β-lactoglobulin in the cooled protein solution 40 have a molecular weight on the order of about 200,000 Daltons. Thus, FIG. 4 demonstrates that, after heating of the alkaline intermediate in accordance with the heating details of this Example, polymers of α-lactalbumin and β-lactoglobulin are present in the cooled protein solution 40, these polymers of α-lactalbumin and β-lactoglobulin are soluble in the cooled protein solution 40 (as evidenced by the presence of the large void volume peak), and, consequently, very little α-lactalbumin or β-lactoglobulin remains in the cooled protein solution 40.

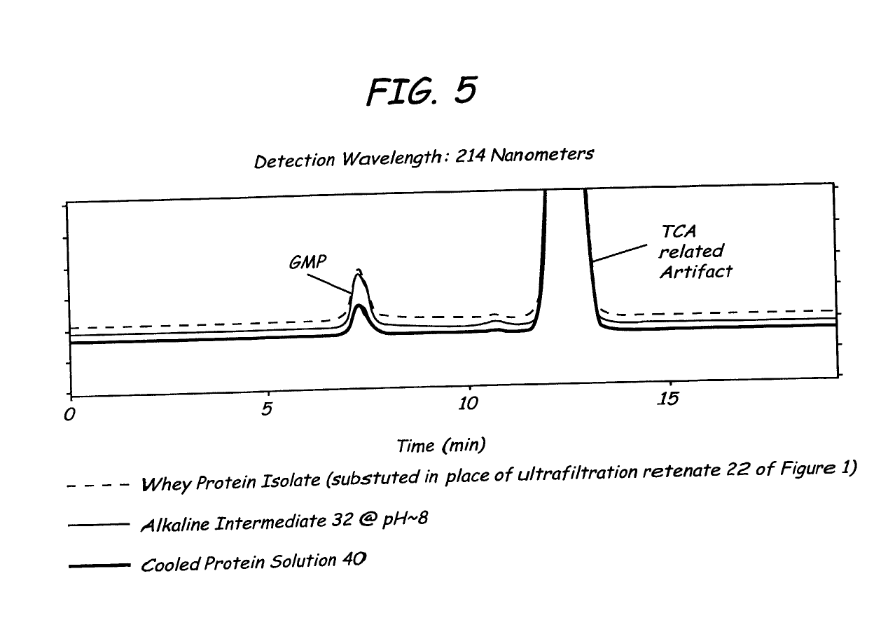

FIG. 5 demonstrates that the glycomacropeptide concentration remained relatively steady both in the feed (whey protein isolate), the alkaline intermediate 32, and the cooled protein solution 40. The slightly diminished peak for the glycomacropeptide concentration in the cooled protein solution 40 is believed due to the addition of dilution water via the culinary steam that was used to heat the intermediate 36. Nonetheless, it is clear that the alkaline conditions and the heat treatment applied to the intermediate 36 causes little if any decrease in the glycomacropeptide component. The large peak to the right of the GMP peak in each sample trace of FIG. 5 is a TCA artifact related to residuals of the TCA used to precipitate any β-lactoglobulin (and any α-lactalbumin) present in the sample, prior to analysis for glycomacropeptide at the 214 nanometer detection wavelength.

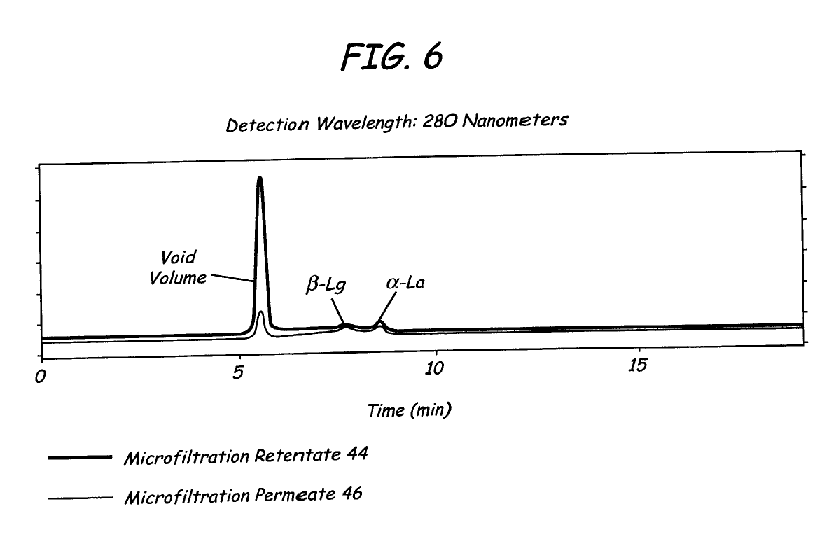

In FIG. 6, details about the microfiltration retentate 44 and the microfiltration permeate 46 are shown. These details of FIG. 6 were obtained after about 60 gallons of the cooled protein solution 40 had been microfiltered. These details of FIG. 6 demonstrate that very little α-lactalbumin or β-lactoglobulin is present in either the microfiltration retentate 44 or the microfiltration permeate 46, which indicates that most of the α-lactalbumin and β-lactoglobulin has been polymerized.

Also, in FIG. 6, the void volume peak for the microfiltration retentate 44 is significantly larger than the void volume peak of the microfiltration permeate 46. The large void volume peak for the microfiltration retentate 44 sample demonstrates that (1) a substantial concentration of polymers of α-lactalbumin and β-lactoglobulin is present in the microfiltration retentate 44 and (2) these polymers of α-lactalbumin and β-lactoglobulin are soluble in the cooled protein solution 40 (as evidenced by the presence of the large void volume peak); consequently, as evidenced by the small α-lactalbumin or β-lactoglobulin peaks in FIG. 6, very little α-lactalbumin or β-lactoglobulin remains in the microfiltration retentate 44. Correspondingly, the small void volume peak for the microfiltration permeate 46 sample demonstrates that (1) only a small concentration of polymers of α-lactalbumin and β-lactoglobulin is present in the microfiltration permeate 46 and (2) these few polymers of α-lactalbumin and β-lactoglobulin are soluble in the cooled protein solution 40 (as evidenced by the presence of the small void volume peak); furthermore, as evidenced by the small α-lactalbumin or β-lactoglobulin peaks in FIG. 6, very little α-lactalbumin or β-lactoglobulin remains in the microfiltration permeate 46.

Thus, FIG. 6 illustrates that the microfiltration excluded the polymerized proteins (polymers of α-lactalbumin and β-lactoglobulin), since the polymerized protein concentration of the microfiltration retentate 44 is much larger than the polymerized protein concentration of the microfiltration permeate 46, as evidenced by the much larger void volume peak of the microfiltration retentate 44 sample as compared to the small void volume peak of the microfiltration permeate 46 sample. Furthermore, FIG. 6 illustrates that very little non-polymerized α-lactalbumin and very little non-polymerized β-lactoglobulin is present in either the microfiltration retentate 44 or the microfiltration permeate 46.

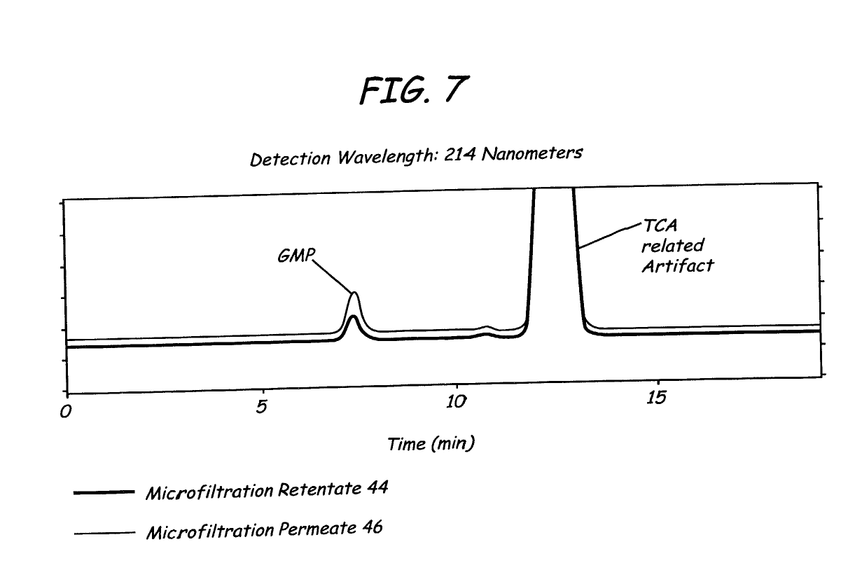

Finally, FIG. 7, which is based upon streams that had been microfiltered, but prior to any diafiltration, demonstrates that the concentration of glycomacropeptide is nevertheless significantly larger in the microfiltration permeate 46, as compared to the concentration of glycomacropeptide in the microfiltration retentate 44. The large peak to the right of the GMP peak in each sample trace of FIG. 7 is a TCA artifact related to residuals of the TCA used to precipitate any β-lactoglobulin (and any α-lactalbumin) present in the sample, prior to analysis for glycomacropeptide at the 214 nanometer detection wavelength.

Ten gallons of the microfiltration permeate 46 was prepared for ultrafiltration. First, the pH of the microfiltration permeate 46 was adjusted to about 6.2 using a 10 weight percent solution of phosphoric acid. The ultrafiltration unit that was used to concentrate the microfiltration permeate 46 was a bench top ultrafilter. The bench top ultrafilter dewatered the microfiltration permeate 46 to form the ultrafiltration retentate 54 and the ultrafiltration permeate 56, where the ultrafiltration permeate 56 primarily consisted of water, along with a small amount of lactose, glycomacropeptide, lactose, minerals, and ash.

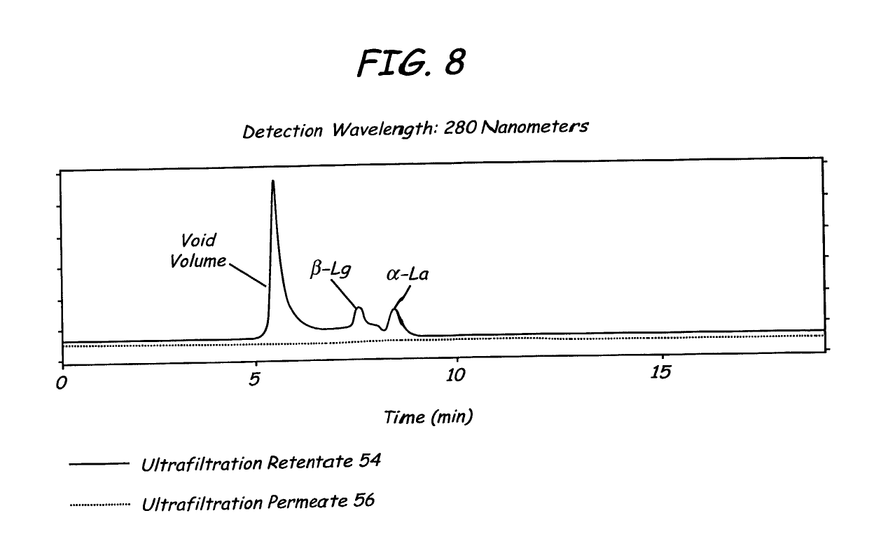

Details about the α-lactalbumin and β-lactoglobulin contents of the ultrafiltration retentate 54 and the ultrafiltration permeate 56 derived from ultrafiltration of the microfiltration permeate 46 are provided in FIG. 8. As noted above, with reference to FIG. 6, very little non-polymerized α-lactalbumin and very little non-polymerized β-lactoglobulin is present in the microfiltration permeate 46, and only a small concentration of polymerized proteins (polymers of α-lactalbumin and β-lactoglobulin) is present in the microfiltration permeate 46. From FIG. 8, it is evident that all of the α-lactalbumin and all of the β-lactoglobulin present in the microfiltration permeate 46 that was ultrafiltered in the bench top ultrafilter was retained in the ultrafiltration retentate 54. Furthermore, FIG. 8 illustrates that the bench top ultrafilter excluded the small concentration of polymerized proteins (polymers of α-lactalbumin and β-lactoglobulin) present in the microfiltration permeate 46, as evidenced by the void volume peak of the ultrafiltration retentate 54 sample as compared to the lack of any void volume peak for the ultrafiltration permeate 56 sample.

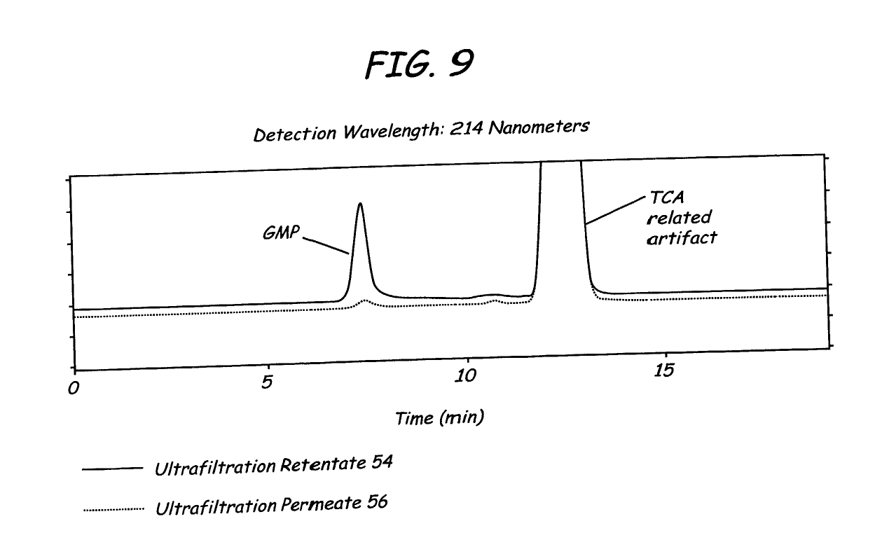

Next, from FIG. 9, it is evident that the majority of glycomacropeptide present in the microfiltration permeate 46 that was fed to the bench top ultrafilter was retained in the ultrafiltration retentate 54, though a very small amount of glycomacropeptide apparently passes through to the ultrafiltration permeate 56. The large peak to the right of the GMP peak in each sample trace of FIG. 9 is a TCA artifact related to residuals of the TCA used to precipitate any β-lactoglobulin (and any α-lactalbumin) present in the sample, prior to analysis for glycomacropeptide at the 214 nanometer detection wavelength.

### Example 2

In this Example, experimentation was directed to evaluation of conditions and variables, if any, that exert control over denaturization of α-lactalbumin, β-lactoglobulin, and glycomacropeptide. Four variables were evaluated in this Example: pH, calcium concentration (as measured by calcium molarity), temperature, and duration of heating.

The data that were obtained in this Example was analyzed using the general linear model (GLM) statistical procedure of SAS® statistical analysis software for a model statement based upon a Box-Behnken experimental design that included variation of two of the four variables identified above. The SAS® statistical analysis software is available from SAS® Institute Inc. of Cary, N.C. Additionally, all data were analyzed to determine coefficients and probabilities for the various models derived from the statistical results.

In this Example, sweet cheese whey that was derived from the manufacture of a cheddar cheese was processed to form a whey protein isolate containing approximately 27 weight percent protein (as total protein), based upon the total weight of the weight protein isolate. The whey protein isolate was immediately cooled to about 40° F. after production and was placed in portable tanks. The portable tanks were held at about 40° F. prior to further processing of the whey protein isolate. The pH of the whey protein isolate, immediately prior to initiation of the present Example, ranged from about 6.2 to about 6.4 standard pH units, which indicates that good handling practices were observed.

The whey protein isolate was diluted with water to form eight liters of a whey protein isolate solution that contained about 3 weight percent total protein, based upon the total weight of the whey protein isolate (WPI) solution. Two liters of the 3 percent WPI solution were adjusted to pH 4.5±0. 1, four liters of the 3 percent WPI solution were adjusted to pH 6.0±0.1, and two liters of the 3 percent WPI solution were adjusted to pH 7.5±0.1. Two hundred milliliter portions of the three WPI solutions with different pHs were placed in 500 milliliter flasks, and calcium chloride was added as appropriate to provide calcium molarities in the different flasks of 0 M calcium, 0.1 M calcium, and 0.2 M calcium. After any calcium addition, the pHs of the different 3 percent WPI solutions in the flasks were readjusted, as necessary, back to the original pH of 4.5, 6.0, or 7.5, as appropriate.

A shaking water bath was provided. The shaking water bath was warmed to either 160° F., 170° F., or 180° F., depending upon the particular combination of variables being evaluated. Each flask was allowed to rest in the shaking water bath for about 15 seconds to reach the particular temperature being evaluated before timing in the shaking water bath was initiated. The flasks that were placed in the shaking water bath were then timed to allow reaction at the selected temperature for either 2 minutes, 4 minutes, or 6 minutes. After the selected reaction time at the selected temperature was completed, the different flasks were placed in an ice bath with agitation to cool the solution and end the reaction.

After cooling in the ice bath, the flasks were placed in a centrifuge for 10 minutes at a rate of 10,000 revolutions per minute at a force of 15,000 g. After being centrifuged, the supernatant in each test flask was decanted through cheese cloth and analyzed for protein concentration. The protein concentration analysis procedures included HPLC analysis for both α-lactalbumin and β-lactoglobulin, which were individually determined, and HPLC analysis (after trichloracetic acid addition for precipitation of any susceptible proteins) for glycomacropeptide.

Details are provided in Table 6 below for the different combinations of the four variables (calcium molarity, pH, reaction time, and reaction temperature) along with the β-lactoglobulin, α-lactalbumin, and glycomacropeptide analysis results for the different runs under consideration:

TABLE 6

| Run | Calcium | | Time | Temp | μ-Lg | α-La | GMP |
|---|---|---|---|---|---|---|---|
| ID | (molarity) | pH | (minutes) | (° F.) | (mg/ml) | (mg/ml) | (mg/ml) |
| DB | 0.1 | 4.5 | 2 | 170 | 15.74 | 3.75 | 0.69 |
| CR | 0.1 | 4.5 | 4 | 160 | 15.13 | 3.63 | 0.82 |
| GD | 0 | 4.5 | 4 | 170 | 14.69 | 1.86 | 0.76 |
| DD | 0.2 | 4.5 | 4 | 170 | 14.84 | 3.67 | 0.79 |
| FB | 0.1 | 4.5 | 4 | 180 | 14.8 | 3.54 | 0.83 |
| GE | 0.1 | 4.5 | 6 | 170 | 14.95 | 3.51 | 0.83 |
| EA | 0.1 | 6 | 2 | 160 | 16.47 | 3.82 | 0.82 |
| AC | 0 | 6 | 2 | 170 | 13.72 | 3.35 | 0.85 |
| DC | 0.2 | 6 | 2 | 170 | 16.11 | 3.85 | 0.82 |
| FA | 0.1 | 6 | 2 | 180 | 16.55 | 3.82 | 0.83 |
| EB | 0 | 6 | 4 | 160 | 16.59 | 3.75 | 0.84 |
| CA | 0.2 | 6 | 4 | 160 | 16.15 | 3.82 | 0.72 |
| AE | 0.1 | 6 | 4 | 170 | 15.19 | 3.65 | 0.82 |
| DA | 0.1 | 6 | 4 | 170 | 16.35 | 3.8 | 0.82 |
| GB | 0.1 | 6 | 4 | 170 | 16.39 | 3.85 | 0.81 |
| FC | 0 | 6 | 4 | 180 | 9.41 | 3.43 | 0.86 |
| BB | 0.2 | 6 | 4 | 180 | 14.58 | 3.69 | 0.82 |
| CC | 0.1 | 6 | 6 | 160 | 16.24 | 3.76 | 0.84 |
| GA | 0 | 6 | 6 | 170 | 11.81 | 3.1 | 0.76 |
| GC | 0.2 | 6 | 6 | 170 | 15.19 | 3.83 | 0.82 |
| BA | 0.1 | 6 | 6 | 180 | 9.72 | 2.9 | 0.86 |
| AB | 0.1 | 7.5 | 2 | 170 | 16.04 | 3.75 | 0.83 |
| EC | 0.1 | 7.5 | 4 | 160 | 15.2 | 3.8 | 0.79 |
| AD | 0 | 7.5 | 4 | 170 | 16.62 | 3.69 | 0.84 |
| AA | 0.2 | 7.5 | 4 | 170 | 9.72 | 2.92 | 0.81 |
| BC | 0.1 | 7.5 | 4 | 180 | 2.31 | 2.53 | 0.89 |
| DE | 0.1 | 7.5 | 6 | 170 | 5.95 | 2.79 | 0.86 |

Next, Table 7 includes details about which variables were varied and the direction of variation for each of the different test runs:

TABLE 7

| Run ID | PHA | CALCIUM | TIME | TEMPERATURE |
|---|---|---|---|---|
| DB | −1 | 0 | −1 | 0 |
| CB | −1 | 0 | 0 | −1 |
| GD | −1 | −1 | 0 | 0 |
| DD | −1 | 1 | 0 | 0 |
| FB | −1 | 0 | 0 | 1 |
| GE | −1 | 0 | 1 | 0 |
| EA | 0 | 0 | −1 | −1 |
| AC | 0 | −1 | −1 | 0 |
| DC | 0 | 1 | −1 | 0 |
| FA | 0 | 0 | −1 | 1 |
| EB | 0 | −1 | 0 | −1 |
| CA | 0 | 1 | 0 | −1 |
| AE | 0 | 0 | 0 | 0 |
| DA | 0 | 0 | 0 | 0 |
| GB | 0 | 0 | 0 | 0 |
| FC | 0 | −1 | 0 | 1 |
| BB | 0 | 1 | 0 | 1 |
| CC | 0 | 0 | 1 | −1 |
| GA | 0 | −1 | 1 | 0 |
| GC | 0 | 1 | 1 | 0 |
| BA | 0 | 0 | 1 | 1 |
| AB | 1 | 0 | −1 | 0 |
| EC | 1 | 0 | 0 | −1 |
| AD | 1 | −1 | 0 | 0 |
| AA | 1 | 1 | 0 | 0 |
| BC | 1 | 0 | 0 | 1 |
| DE | 1 | 0 | 1 | 0 |

Finally, coefficients and probabilities for the various models derived from the results are provided in Table 8 below:

TABLE 8

| Dependent | βLg | | α-La | | GMP | |
|---|---|---|---|---|---|---|
| Variable | Coefficient | Probability | Coefficient | Probabiliti | Coefficient | Probability |
| r2 | 0.83 | | 0.81 | | 0.61 | |
| Intercept | 16.00 | 0 | 3.77 | 0 | 0.81 | |
| CAL | | | 0.22 | 0.035 | | |
| PHA | −2.03 | 0.007 | | | −0.03 | 0.048 |
| TIM | −1.73 | 0.018 | −0.20 | 0.045 | | |
| TEM | −2.37 | 0.0027 | −0.22 | 0.031 | 0.02 | 0.08 |
| CAL*CAL | | | | | | |
| PHA*CAL | | | −0.65 | 0.0015 | | |
| PHA*PHA | −2.18 | 0.039 | −0.39 | 0.014 | | |
| TIM*PHA | −2.33 | 0.054 | | | | |
| TIM*TIM | | | | | | |
| TEM*CAL | | | | | | |
| TEM*PHA | −3.14 | 0.014 | −0.30 | 0.087 | | |
| TEM*TIM | | | | | | |
| TEM*TEM | | | | | | |

The data of Table 6 support a conclusion that addition of calcium during the reaction does not support enhancement of β-lactoglobulin denaturization under the particular test variables and conditions chosen for this Example. Also, calcium addition appears to provide a slight protective effect to α-lactalbumin that helps prevent, at least to some extent, denaturization of α-lactalbumin.

It was generally expected that β-lactoglobulin would be more highly denatured at lower pHs, such as at the pH conditions of 4.5. Surprisingly, however, it appears that β-lactoglobulin is relatively stable at pHs of about 4.5, but can be more easily denatured at pHs of about 7.5. Furthermore, at higher temperatures, pHs greater than about 6 appear to increase β-lactoglobulin denaturization more than a combination of higher temperatures and a pH lower than about 6. Also, increased reaction times appear to contribute to increased β-lactoglobulin denaturization. Thus, higher pHs, increased reaction times, and higher reaction temperatures all appear to contribute to increased denaturization of β-lactoglobulin.

On the other hand, increased reaction times and increased reaction temperatures appear to be primarily responsible for increased α-lactalbumin denaturization. Changes in pH appear to affect α-lactalbumin denaturization only in the higher order terms, or thus only when other variables, such as time and temperature, are changed. Finally, calcium concentrations and pH changes have no effect on the ability to denature glycomacropeptide under the particular test variables and conditions chosen for this Example. Additionally, changes in reaction time and changes in reaction temperature appear to have only a slight effect on the ability to denature glycomacropeptide.

Thus, based upon the results of this Example, reaction conditions at higher pHs, such as in the alkaline regime; increased reaction time; and increased reaction temperature are indicated for purposes of causing some degree of denaturization of both α-lactalbumin and β-lactoglobulin, while either not denaturing or only insignificantly denaturing glycomacropeptide. Also, based upon the non-influence of calcium on β-lactoglobulin and glycomacropeptide denaturization and the very slight effect of calcium concentration on α-lactalbumin denaturization, there is apparently no need to modify calcium concentrations within the ranges tested in this experiment for purposes of controlling denaturization of α-lactalbumin, β-lactoglobulin, and glycomacropeptide.

Consequently, based upon the test results of this Example, it has been surprisingly found that there are definite reaction variables that are applicable to a solution containing α-lactalbumin, β-lactoglobulin, and glycomacropeptide that will support denaturization of (i.e.: polymerization) α-lactalbumin and β-lactoglobulin while avoiding or predominantly avoiding any denaturization of glycomacropeptide. Thus, with this knowledge, it is possible to cause polymerization of α-lactalbumin and β-lactoglobulin to provide a polymerized protein fraction with beneficial functional attributes created by the polymerization, while leaving glycomacropeptide in a non-denatured state that supports capture of the beneficial biological properties of non-denatured glycomacropeptide. Furthermore, this ability to choose conditions that support formation of larger, polymers of α-lactalbumin and β-lactoglobulin, while retaining glycomacropeptide as smaller non-denatured particles provides, an avenue for efficiently separating the beneficial polymers of α-lactalbumin and β-lactoglobulin from the smaller non-denatured particle forms of glycomacropeptide.

### Example 3

This Example demonstrates processing of a starting dairy material that contains glycomacropeptide, α-lactalbumin, and β-lactoglobulin to produce a polymerized protein powder that is rich in polymers of α-lactalbumin and the β-lactoglobulin and a GMP powder that is rich in glycomacropeptide. One goal of this Example was to maximize recovery of glycomacropeptide in the microfiltration permeate 46, as opposed to either maximizing production of polymers of both β-lactoglobulin and α-lactalbumin or maximizing recovery of polymerized proteins in the microfiltration retentate 44.

In this Example, sweet cheese whey that was derived from the manufacture of a cheddar cheese was processed to form 80% whey protein concentrate containing approximately 80 weight percent total solids, based upon the total weight of the 80% whey protein concentrate. The 80% whey protein concentrate was immediately cooled to about 40° F. after production and was placed in 240 gallon portable tanks. The portable tanks were held at about 40° F. prior to further processing of the 80% whey protein concentrate. The pH of the 80% whey protein concentrate was in the range from about 6.2 to about 6.4 standard pH units, which indicates that good handling procedures were observed.

Subsequent aspects of this Example were conducted in accordance with the schematic drawing of FIG. 1, where the 80% whey protein concentrate is substituted in place of the ultrafiltration retentate 22. Fifteen gallons of the 80% whey protein concentrate (which served as the ultrafiltration retentate 22 in this Example) were diluted with 75 gallons of reverse osmosis water (dilution water 26) to produce the diluted intermediate 28 with a concentration of about 5 weight percent total protein, based upon the total weight of the diluted intermediate 28.

The total volume of the diluted intermediate 28 was about 90 gallons.

Ten weight percent sodium hydroxide, as the alkaline agent 30, was added to the diluted intermediate 28 to yield the alkaline intermediate 32 with a pH of about 7.7. The alkaline intermediate 32 was passed, in continuous fashion, through a high temperature short time (HTST) plate heat exchanger at a rate of about 4.6 gallons of the alkaline intermediate 28 per minute. The temperature of the intermediate 32 upon exiting the HTST plate heat exchanger was about 177° F. This heating of the intermediate 32 transformed the alkaline intermediate 32 into the heated intermediate 36. The heated intermediate 36 was collected in a vat and held for about 120 seconds before being cooled to a temperature of about 117° F. to form the cooled protein solution 40. The cooling of the intermediate 36 was accomplished using a cooling water jacket that surrounded the vat containing the intermediate 36.

The cooled protein solution 40 was then microfiltered in the microfilter 42, without adjusting the pH of the solution 40. The microfilter 42 consisted of an AF-1000 microfiltration membrane structure obtained from PTI Advanced Filtration, Inc. of San Diego, Calif. that was positioned in a microfiltration canister. The membrane of the microfiltration membrane structure was manufactured of polyvinyl difluoride (PVDF) with a molecular weight cut-off (MWCO) of about 1,000,000 Daltons and a nominal pore diameter on the order of about 0.1 microns. The AF-1000 membrane structure included about 17-18 spirals of the membrane sheet. The distance between facing spirals of the membrane sheet was about 65 mils.

The cooled protein solution 40 was concentrated to minimum volume via batch filtration, flowed by batch diafiltration, in the microfilter 42. As a result of the batch filtration in the microfilter 42, the volume of the microfiltration retentate 44 in this Example was concentrated to about 20 gallons versus the 75 gallons of the cooled protein solution 40 that were used as feed to the microfilter 42. After microfiltration of the cooled protein solution 40, the microfiltration retentate 44 was then batch diafiltered three times in the microfilter 42 using about 20 gallons of reverse osmosis water per batch diafiltration.

The microfilter 42, during both the initial filtration and the subsequent diafiltration, was operated to maintain about 8 psig inlet pressure to the membrane structure and the back pressure on the microfiltration retentate 44 was about 3 psig. The temperature of the cooled protein solution 40 during the microfiltration and the temperature of the feed during the subsequent diafiltration were each maintained in the range of about 111° F. to about 115° F. The diafiltered microfiltration retentate 44 was collected and spray dried to form polymerized protein powder 50. The microfiltration permeate 46 that was obtained during the initial microfiltration of the cooled protein solution 40 was combined with the microfiltration permeate 46 obtained during diafiltration of the microfiltration retentate 44. This combined microfiltration permeate 46 stream totaled about 110 gallons.

The pH of the 110 gallons of the combined microfiltration permeate 46 was adjusted to about 6.5 by addition of 10 weight percent phosphoric acid with stirring. The phosphoric acid was added very slowly and a precipitate formed at the site of the addition. However, this precipitate readily redissolved upon additional stirring of mixture. The acidified form of the combined microfiltration permeate 46 was ultrafiltered using a Romicon PM5 membrane structures that had an MWCO of 5,000 Daltons. The membrane of the Romicon PM5 membrane structures was made of polysulfone and had a hollow fiber design. The Romicon PM5 membrane may be obtained from Koch Membrane Systems, Inc. of Wilmington, Mass.

The ultrafiltration system was operated in batch mode at all times with the retentate from the Romicon membrane structure recycled back through the Romicon membrane structure until the total volume of ultrafiltration retentate 54 obtained by ultrafiltering the original 110 gallons of the acidified form of the combined microfiltration permeate 46 was about 20 gallons. The 20 gallons of ultrafiltration retentate 54 was thereafter spray dried to form a glycomacropeptide (GMP) powder.

The inlet pressure on the feed to the Romicon PM5 structure was maintained in the range from about 40 pounds psig to about 120 psig, and the discharge pressure on the ultrafiltration retentate from the Romicon PM5 structure was maintained about 0 psig and about 60 psig, with the caveat that the difference between the inlet pressure on the feed to the Romicon PM5 structure and the discharge pressure on the ultrafiltration retentate was greater than 0 psig. The temperature of the feed (acidified form of the combined microfiltration permeate 46) to, and in, the Romicon PM5 structure was maintained at about 115° F. while the ultrafiltration occurred.

Details about the component weights for the 80% whey protein concentrate (which served as the ultrafiltration retentate 22 in this Example); the intermediates 28, 32; the cooled protein solution 20; the microfiltration retentate 44; the microfiltration permeate 46; the polymerized protein powder 50; and the glycomacropeptide powder that was derived by spray drying the acidified ultrafiltration retentate 54 are provided in Table 9 below:

TABLE 9

| | Total | Total | Total | Total | | | |
|---|---|---|---|---|---|---|---|
| | Solids | Protein | Fat | Ash | β-lg* | α-la** | GMP*** |
| Stream Description | (lbs.) | (lbs.) | (lbs.) | (lbs.) | (lbs.) | (lbs.) | (lbs.) |
| Diluted 80% WPC (as UF 1 retentate 22) | 36.06 | 29.22 | 2.32 | 1.10 | 16.07 | 3.96 | 3.34 |
| Alkaline (pH˜7.7) Intermediate 32 | 36.12 | 29.22 | NM | NM | 16.11 | 3.99 | 3.19 |
| Cooled Protein Solution 40 | 36.18 | 29.15 | 2.13 | 1.23 | 4.59 | 1.95 | 3.08 |
| Microfiltration Retentate 44 | 17.72 | 15.02 | 2.37 | 0.28 | 0.01 | 0.04 | 0.00 |
| Microfiltration Permeate 46 | 17.50 | 13.24 | <0.09 | 1.14 | 4.32 | 1.66 | 3.07 |
| Polymerized Protein Powder 50 | 9.77 | 8.26 | 1.29 | 0.16 | | | |
| Glycomacropeptide Powder | 7.90 | 7.07 | 0.04 | 0.32 | | | |

*β-lactoglobulin
**α-lactalbumin
***glycomacropeptide

These details of Table 9 demonstrate that more than 90 percent of the GMP originally present in the diluted 80% Whey Protein Concentrate survives the heating step. These details of Table 9 also demonstrate that the polymerized protein powder 50 is, on a weight basis, about 80% protein (as total protein), about 12.5% fat, and about 1.5% ash, based upon the total weight of the polymerized protein powder 50. These details of Table 9 also demonstrate that the glycomacropeptide powder is, on a weight basis, about 84% protein (as total protein), about 0.5% fat, and about 4% ash, based upon the total weight of the glycomacropeptide powder.

The total solids, total protein, total fat, and total ash weights presented in Table 9 were calculated, using the total weight of the particular stream, after determining the weight percent total solids, weight percent total protein, weight percent total fat, and weight percent total ash of the different streams using the procedures for these particular determinations that are described in the Property Determination and Characterization Techniques section of this document. Also, the β-lactoglobulin, α-lactalbumin, and glycomacropeptide weights presented in Table 9 were calculated, using the total volume of the particular stream, after determining the weight-based concentration (mg/ml) of β-lactoglobulin, α-lactalbumin, and glycomacropeptide of the different streams using Protein Analysis Procedure No. 1 that is described in the Property Determination and Characterization Techniques section of this document, with the exception that a pair of 300 mm×7.8 mm Bio-Sil SEC 125 size exclusion columns were joined in series and substituted for the single 300 mm×7.8 mm Bio-Sil SEC 125 size exclusion column prescribed in Protein Analysis Procedure No. 1.

Minor discrepancies between the total solids and the total protein present in the combination of the microfiltration retentate 44 and the microfiltration permeate 46 versus the total solids and the total protein in the intermediates 28, 32 and the cooled protein solution 40, respectively, is believed due, in large part at least, to residual amounts of insoluble proteins that remained following transfer of the cooled protein solution 40 to the microfilter 42. The decreasing ratios of the combined weighs of β-lactoglobulin, α-lactalbumin, and glycomacropeptide for a particular stream to the total protein weight for the particular stream, when going from the intermediates 28, 32, to the cooled protein solution 40, to the microfiltration retentate 44, and to the microfiltration permeate 46 reflects the desired loss of solubility of β-lactoglobulin and α-lactalbumin, upon heating of the intermediate 32. Also, as seen in Table 9, the split between the total protein content of the microfiltration retentate 44 and the total protein content of the microfiltration permeate 46 is less pronounced than in Example 1, since the focus of this Example is to maximize recovery of glycomacropeptide in the microfiltration permeate 46, as opposed to maximizing production of polymers of β-lactoglobulin and α-lactalbumin and maximizing recovery of polymerized proteins in the microfiltration retentate 44.

Next, percent recovery data for total solids, total protein, total fat, total ash, β-lactoglobulin, α-lactalbumin, and glycomacropeptide ranging from the intermediates 30, 32, to the cooled protein solution 40, to the microfiltration retentate 44 and the microfiltration permeate 46, and ending with the polymerized protein powder 50 and the glycomacropeptide powder that was derived by spray drying the acidified ultrafiltration retentate 54 were calculated from the weight data of Table 9 and are provided in Table 10 below:

TABLE 10

| | Total | Total | Total | | | | |
|---|---|---|---|---|---|---|---|
| | Solids | Protein | Fat | Total Ash | β-lg* | α-la** | GMP*** |
| Stream Description | (% Rec.)^{A} | (% Rec.)^{A} | (% Rec.)^{A} | (% Rec.)^{A} | (% Rec.)^{A} | (% Rec.)^{A} | (% Rec.)^{A} |
| Diluted 80% WPC (as UF 1 retentate 22) | 100 | 100 | 100 | 100 | 100 | 100 | 100 |
| Cooled Protein Solution 40 | 100 | 100 | 92 | 112 | 29 | 49 | 92 |
| Microfiltration Retentate 44 | 49 | 52 | 112 | 22 | 0 | 1 | 0 |
| Microfiltration Permeate 46 | 48 | 45 | <4 | 93 | 27 | 42 | 92 |
| Polymerized Protein Powder 50 | 27 | 28 | 60 | 13 | | | |
| Glycomacropeptide Powder | 22 | 24 | 2 | 26 | | | |

*β-lactoglobulin
**α-lactalbumin
***glycomacropeptide
^{A}Percent recovery of the particular component in a particular stream versus the amount of the component originally present in the diluted 80% WPC (as UF 1 retentate 22).

The data of Table 10 demonstrate that almost all of the glycomacropeptide remained soluble in the cooled protein solution 40 following the described heating. Also, almost one quarter of the β-lactoglobulin and almost one half of the α-lactalbumin remained soluble in the cooled protein solution 40 following the described heating. However, this latter observation is consistent with the focus of this Example to maximize recovery of glycomacropeptide in the microfiltration permeate 46, as opposed to maximizing production of polymers of β-lactoglobulin and α-lactalbumin and maximizing recovery of polymerized proteins in the microfiltration retentate 44.

Next, the concentrations of β-lactoglobulin, α-lactalbumin, glycomacropeptide, Immunoglobulin G, and bovine serum albumin were determined using Protein Analysis Procedure No. 1 that is described in the Property Determination and Characterization Techniques section of this document for an aqueous solution of the glycomacropeptide powder that had been derived by spray drying the acidified ultrafiltration retentate 54. In this HPLC determination, the single 300 mm×7.8 mm Bio-Sil SEC 125 size exclusion column prescribed in Protein Analysis Procedure No. 1 was used. The aqueous solution that was analyzed was prepared by blending 512.4 milligrams of the glycomacropeptide powder with 100 milliliters of water to yield the aqueous solution with a concentration of 5.12 milligrams of glycomacropeptide per milliliter of water.

The Total Protein determination procedure from the Property Determination and Characterization Techniques section of this document was employed to determine that the glycomacropeptide powder of this Example was about 84.17 weight percent protein (as total protein), based upon the total weight of the glycomacropeptide powder. The concentrations of β-lactoglobulin, α-lactalbumin, glycomacropeptide, Immunoglobulin G, and bovine serum albumin in the aqueous solution of the glycomacropeptide powder were 1.52, 0.75, 2.05, 0, and 0 milligrams per milliliter (mg/ml) of the aqueous solution, respectively. Using these concentration details and the 84.17 weight percent protein (as total protein) known to be in the glycomacropeptide powder, the weight percentages of β-lactoglobulin, α-lactalbumin, and glycomacropeptide relative to the total weight of the glycomacropeptide powder (as-is basis) and the weight percentages of β-lactoglobulin, α-lactalbumin, and glycomacropeptide relative to each other (protein basis) were determined and are presented in Table 11 below:

TABLE 11

| | β-lg* | α-la** | GMP*** |
|---|---|---|---|
| Stream Description | (weight %) | (weight %) | (weight %) |
| Glycomacropeptide Powder | 30 | 15 | 40 |
| (“as is” basis^{A}) | | | |
| Glycomacropeptide Powder | 35 | 17 | 48 |
| (“protein” basis^{B}) | | | |

*β-lactoglobulin
**α-lactalbumin
***glycomacropeptide
^{A}as a percentage of the total weight of the glycomacropeptide powder
^{B}as a percent of the total weight of β-lactoglobulin, α-lactalbumin, and glycomacropeptide present in the glycomacropeptide powder

Thus, the data of Table 11 demonstrate that the glycomacropeptide powder produced in this Example was about 40 weight percent glycomacropeptide, based upon the total weight of the glycomacropeptide powder. Additional, this data demonstrate that about one half of the protein in the glycomacropeptide powder of this Example was glycomacropeptide, based upon the weight of the glycomacropeptide and the total weight of the glycomacropeptide powder. Furthermore, the Table 11 data show that glycomacropeptide, β-lactoglobulin, and α-lactalbumin constitute about 100% of the protein present in the GMP powder, while aggregated protein molecules (i.e.: polymers of β-lactoglobulin and α-lactalbumin) constitute little, if any, of the protein present in the GMP powder.

Next, Table 12 provides a comparison of the weight percentages of β-lactoglobulin, α-lactalbumin, and glycomacropeptide relative to each other (protein basis) in both the starting 80% whey protein concentrate that was diluted with water to form the feed (ultrafiltration retentate 22) of this Example and in the glycomacropeptide powder produced in this Example:

TABLE 12

| | β-lg* | α-la** | GMP*** |
|---|---|---|---|
| Stream Description | (weight %) | (weight %) | (weight %) |
| 80% Whey Protein | 69 | 17 | 14 |
| Concentrate (“protein” basis^{A}) | | | |
| Glycomacropeptide powder | 35 | 17 | 48 |
| (“protein” basis^{B}) | | | |

*β-lactoglobulin
**α-lactalbumin
***glycomacropeptide
^{A}as a percent of the total weight of β-lactoglobulin, α-lactalbumin, and glycomacropeptide present in the starting 80% whey protein concentrate
^{B}as a percentage of the total weight of β-lactoglobulin, α-lactalbumin, and glycomacropeptide present in the glycomacropeptide powder

Thus, the data of Table 12 show the concentration of glycomacropeptide in the glycomacropeptide powder was about 3.36 times greater (48÷14) than the concentration of glycomacropeptide in the starting 80% whey protein concentrate that was diluted with water to form the feed (ultrafiltration retentate 22) of this Example.

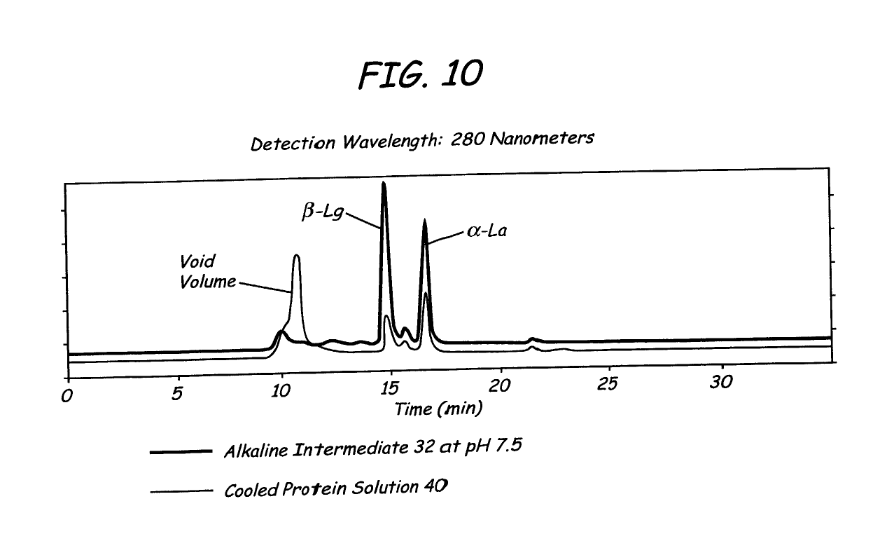

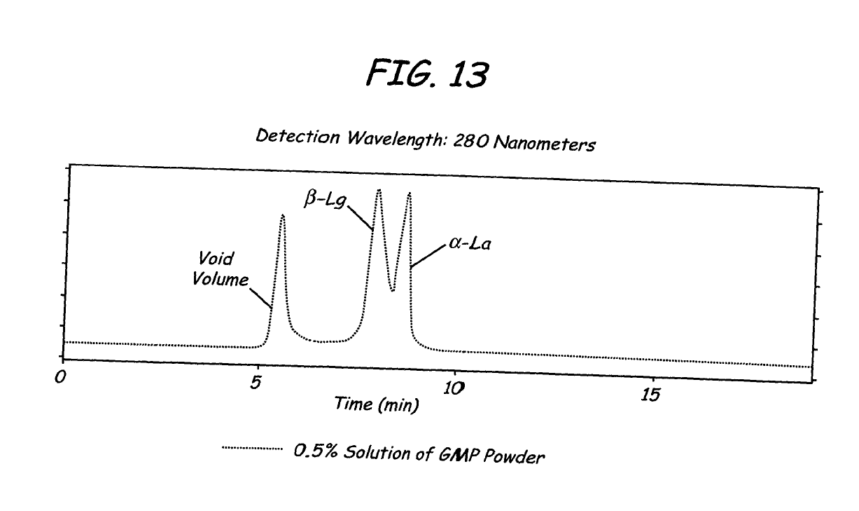

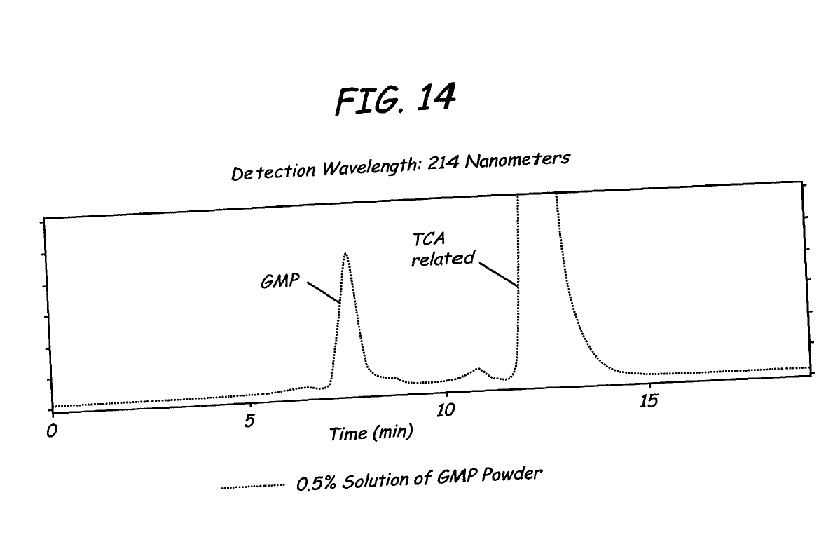

Samples of various streams described above in this Example were subjected to HPLC analysis based upon size-exclusion in accordance with Protein Analysis Procedure No. 1 that is provided above in the Property Determination And Characterization Techniques section of this document. In the HPLC procedure, when producing the scans of FIGS. 10-12, a pair of 300 mm×7.8 mm Bio-Sil SEC 125 size exclusion columns were joined in series and substituted for the single 300 mm×7.8 mm Bio-Sil SEC 125 size exclusion column prescribed in Protein Analysis Procedure No. 1. On the other hand, when producing the scans of FIGS. 13-14, the stationary phase of the chromatographic system was the single 300 mm×7.8 mm Bio-Sil SEC 125 size exclusion column, as specified in Protein Analysis Procedure No. 1, rather than the pair of 300 mm×7.8 mm Bio-Sil SEC 125 size exclusion column, joined in series, that were used when producing the scans of FIGS. 10-12.

As depicted in FIG. 10, the concentrations of both α-lactalbumin and β-lactoglobulin are substantially decreased in the cooled protein solution 40 of this Example versus the concentrations of both α-lactalbumin and β-lactoglobulin in the alkaline intermediate 32 (5% aqueous solution of 80% whey protein concentrate at pH 7.7) of this Example. Furthermore, from FIG. 10, it is clear that the relative concentration of α-lactalbumin is decreased less from the alkaline intermediate 32 to the cooled protein solution 40, as compared to the relative concentration decrease of β-lactoglobulin from the alkaline intermediate 32 to the cooled protein solution 40. On the other hand, in FIG. 10, the void volume peak, which represents polymerized protein, is very large for the cooled protein solution 40, while the void volume peak is relatively small for the alkaline intermediate 32.

In size exclusion HPLC, polymers that are insoluble in the solution (here, an aqueous solution) being analyzed are excluded from the column by filtration frits. Also, in size exclusion HPLC, larger molecules that are soluble in the solution being analyzed and therefore enter the column are less likely to enter the pores of the absorbent gel and are therefore eluted first in the column. The large void volume peak of the cooled protein solution 40 sample represents relatively large molecules, namely polymers of α-lactalbumin and β-lactoglobulin that are formed by sulfur bridging between cysteine residues of the original α-lactalbumin and β-lactoglobulin. The fact that the polymers of α-lactalbumin and β-lactoglobulin represented by the large void volume peak in FIG. 10 are observed in the scan at all indicates that these polymers of α-lactalbumin and β-lactoglobulin, by virtue of entering the HPLC column, are soluble in the aqueous solution being analyzed by the HPLC procedure.

The position of the large void volume peak in the trace of FIG. 10 reflects the size of the polymers of α-lactalbumin and β-lactoglobulin in the cooled protein solution 40. If the position of the large void volume peak were at the very front (leftmost position) of the trace, this would indicate that the polymers of α-lactalbumin and β-lactoglobulin in the cooled protein solution 40 have a molecular weight greater than about 300,000 Daltons. Instead, the position of the large void volume peak in FIG. 10 appears somewhat to the right of the front of the trace. Indeed, the position of the large void volume peak, relative to the positions of the peaks for α-lactalbumin and β-lactoglobulin, respectively, indicates that the polymers of α-lactalbumin and β-lactoglobulin in the cooled protein solution 40 have a molecular weight on the order of about 200,000 Daltons.

Thus, FIG. 10 demonstrates that, after heating of the alkaline intermediate 32 in accordance with the heating details of this Example, polymers of α-lactalbumin and β-lactoglobulin are present in the cooled protein solution 40, these polymers of α-lactalbumin and β-lactoglobulin are soluble in the cooled protein solution 40 (as evidenced by the presence of the large void volume peak), and, consequently, less α-lactalbumin and less β-lactoglobulin remains in the cooled protein solution 40 than was originally present in the alkaline intermediate 32. The diminished reduction of α-lactalbumin and β-lactoglobulin between the alkaline intermediate 32 and the cooled protein solution 40 in this Example versus the reduction experienced in Example 1 is believed due to the different heating approach taken in this Example versus the heating approach taken in Example 1.

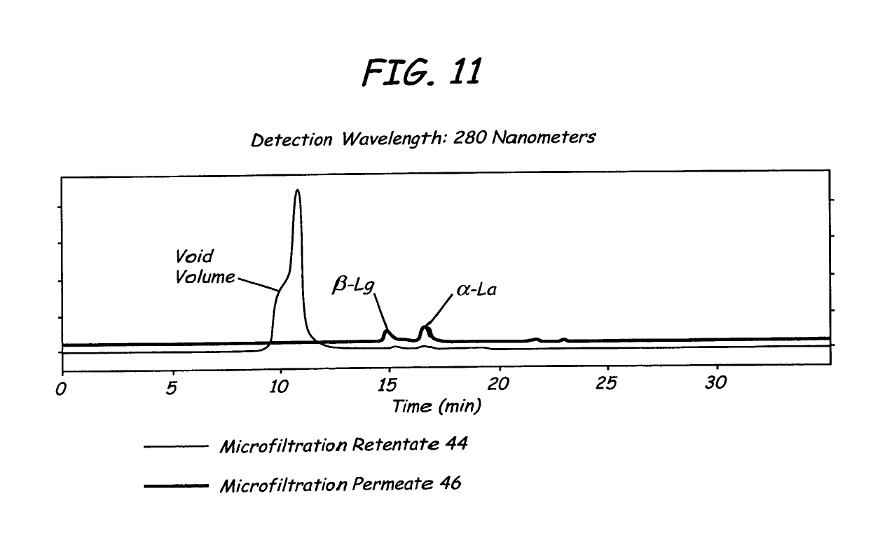

In FIG. 11, details about the microfiltration retentate 44 and the microfiltration permeate 46 are shown. The sample of the microfiltration retentate 44 that was analyzed for FIG. 11 was obtained from the diafiltered microfiltration retentate 44 remaining after both (1) the initial microfiltration of the cooled protein solution 40 and (2) the three stage diafiltration of the initial microfiltration retentate 44. The sample of the microfiltration permeate 46 that was analyzed for FIG. 11 was obtained from the 110 gallon combined microfiltration permeate 46 stream that included both (1) the microfiltration permeate 46 obtained during the initial microfiltration of the cooled protein solution 40 and (2) the microfiltration permeate 46 obtained during the three stage diafiltration of the microfiltration retentate 44.

These details of FIG. 11 show that very little α-lactalbumin or β-lactoglobulin is present in the microfiltration retentate 44, while slightly higher concentrations of α-lactalbumin and β-lactoglobulin are present in the microfiltration retentate 44 as was present in the cooled protein solution 40. Thus, as compared to the very small concentration of α-lactalbumin and β-lactoglobulin remaining in the microfiltration retentate 44 of Example 1, the somewhat larger concentration of α-lactalbumin and β-lactoglobulin remaining in the microfiltration retentate 44 of this Example demonstrates that the different heating approach taken in this Example, versus the heating approach taken in Example 1, caused polymerization of a lower percentage of the α-lactalbumin and β-lactoglobulin originally present in the alkaline intermediate 32 of this Example, as compared to the percentage of α-lactalbumin and β-lactoglobulin polymerized in Example 1.

However, in FIG. 11, the void volume peak for the microfiltration retentate 44 is very large, while virtually no void volume peak is seen for the microfiltration permeate 46. The large void volume peak for the microfiltration retentate 44 sample demonstrates that (1) a substantial concentration of polymers of α-lactalbumin and β-lactoglobulin is present in the microfiltration retentate 44 and (2) these polymers of α-lactalbumin and β-lactoglobulin are soluble in the cooled protein solution 40 (as evidenced by the presence of the large void volume peak); consequently, as evidenced by the very small α-lactalbumin and β-lactoglobulin peaks in FIG. 11, very little α-lactalbumin and very little β-lactoglobulin remains in the microfiltration retentate 44. Correspondingly, the virtually non-existent void volume peak for the microfiltration permeate 46 sample demonstrates that few, if any, polymers of α-lactalbumin and β-lactoglobulin are present in the microfiltration permeate 46.

Thus, FIG. 11 illustrates that the microfiltration excluded the polymerized proteins (polymers of α-lactalbumin and β-lactoglobulin), since the polymerized protein concentration of the microfiltration retentate 44 is much larger than the essentially non-existent polymerized protein concentration of the microfiltration permeate 46, as evidenced by the much larger void volume peak of the microfiltration retentate 44 sample as compared to the virtually non-existent void volume peak of the microfiltration permeate 46 sample. Furthermore, FIG. 11 illustrates that very little non-polymerized α-lactalbumin and very little non-polymerized β-lactoglobulin is present in the microfiltration retentate 44, while a significant concentration of both non-polymerized α-lactalbumin and non-polymerized β-lactoglobulin is present in the microfiltration permeate 46.

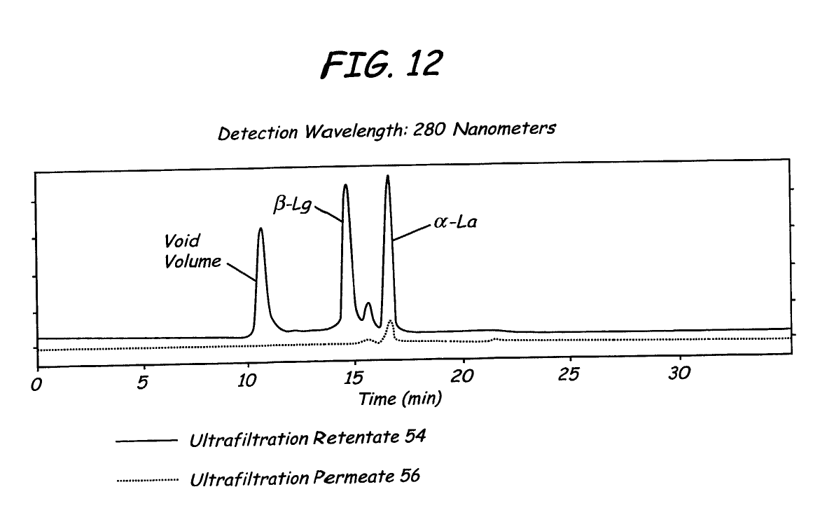

Details about the α-lactalbumin and β-lactoglobulin contents of the ultrafiltration retentate 54 and the ultrafiltration permeate 56 are provided in FIG. 12. The ultrafiltration retentate 54 and the ultrafiltration permeate 56 that were analyzed for FIG. 12 were each obtained from ultrafiltration of the 110 gallon combined microfiltration permeate 46 stream that included both (1) the microfiltration permeate 46 obtained during the initial microfiltration of the cooled protein solution 40 and (2) the microfiltration permeate 46 obtained during the three stage diafiltration of the microfiltration retentate 44.

As noted above, with reference to FIG. 11, very little non-polymerized α-lactalbumin and very little non-polymerized β-lactoglobulin was present in the microfiltration permeate 46, and an essentially non-existent concentration of polymerized proteins (polymers of α-lactalbumin and β-lactoglobulin) was present in the microfiltration permeate 46. These details of FIG. 12 demonstrate that the vast majority of the non-polymerized α-lactalbumin and all of the non-polymerized β-lactoglobulin present in the microfiltration permeate 46 addressed in FIG. 11 remained in the ultrafiltration retentate 54, which is consistent with the details of FIG. 11 that show the microfiltration permeate 46 to include significant concentrations of both non-polymerized α-lactalbumin and non-polymerized β-lactoglobulin. Correspondingly, FIG. 12 illustrates that only a minor amount of the non-polymerized α-lactalbumin and none of the non-polymerized β-lactoglobulin present in the microfiltration permeate 46 addressed in FIG. 11 passed through the ultrafiltration membrane as part of the ultrafiltration permeate 56.

Furthermore, FIG. 12 illustrates that no polymerized proteins (polymers of α-lactalbumin and β-lactoglobulin) passed through the ultrafiltration membrane and into the ultrafiltration permeate 56, as evidenced by the lack of any void volume peak for the ultrafiltration permeate 56. On the other hand, the significant void volume peak for the ultrafiltration retentate 54 sample demonstrates that (1) a significant concentration of polymers of α-lactalbumin and β-lactoglobulin exists in the ultrafiltration retentate 54 and (2) these polymers of α-lactalbumin and β-lactoglobulin are soluble in the ultrafiltration retentate 54 (as evidenced by the presence of the significant void volume peak). The significant concentration of polymers of α-lactalbumin and β-lactoglobulin in the ultrafiltration retentate 54 is interesting, considering that few, if any, polymers of α-lactalbumin and β-lactoglobulin were present in the microfiltration permeate 46 that was ultrafiltered to form the ultrafiltration retentate 54.

A-Lactalbumin and β-lactoglobulin present in the microfiltration permeate 46 are thought to include cysteine residues, and more specifically exposed cysteine sulfhydryl groups. The exposed cysteine sulfhydryl groups are thought to be formed as a result of the denaturing conditions experienced by α-lactalbumin and β-lactoglobulin during the alkaline intermediate 32 heating step and the subsequent elevated temperature maintenance of the heated intermediate 36. It is thought that the polymers of α-lactalbumin and β-lactoglobulin found in the ultrafiltration retentate 54 may be formed during ultrafiltration as a result of oxidation that catalyzes a sulfur bridging reaction between different cysteine residues (i.e. cysteine sulfhydryl groups) of α-lactalbumin and β-lactoglobulin present in the microfiltration permeate 46. Air that is inevitably incorporated into solution during ultrafiltration of the microfiltration permeate 46 supports the oxidation reaction.

In FIG. 13, details about a 0.5 weight percent aqueous solution of the GMP powder, based upon the total weight of the solution, are shown. The GMP powder was formed by spray drying the ultrafiltration retentate 54. These details of FIG. 13 demonstrate that some non-polymerized α-lactalbumin and some non-polymerized β-lactoglobulin is present in the aqueous GMP powder solution. Also, the presence of the significant void volume peak for the ultrafiltration retentate 54 sample demonstrates that (1) polymers of α-lactalbumin and β-lactoglobulin are present in the aqueous GMP powder solution and (2) these polymers of α-lactalbumin and β-lactoglobulin are soluble in the aqueous GMP powder solution (as evidenced by the presence of the significant void volume peak).

Furthermore, the peaks for the non-polymerized α-lactalbumin, non-polymerized β-lactoglobulin, and the void volume peak of the aqueous GMP powder solution have about the same amplitude as the peaks for the non-polymerized α-lactalbumin, non-polymerized β-lactoglobulin, and the void volume peak of the ultrafiltration retentate 54. It is noted that the peaks for the non-polymerized α-lactalbumin and non-polymerized β-lactoglobulin of the aqueous GMP powder solution have somewhat different shapes adjacent to each other, as compared to the peaks for the non-polymerized α-lactalbumin and non-polymerized β-lactoglobulin of the ultrafiltration retentate 54. This apparent difference in shape is believed due solely to the use of a single Bio-Sil SEC 125 size exclusion column for the HPLC analysis of the aqueous GMP powder solution, as compared to the use of two serially-joined Bio-Sil SEC 125 size exclusion columns for the HPLC analysis of the ultrafiltration retentate 54, which would be expected to reduce the resolution of the HPLC trace for the aqueous GMP powder solution versus the resolution of the HPLC trace for the ultrafiltration retentate 54.

In FIG. 14, additional details about the 0.5 weight percent aqueous solution of the GMP powder are shown. These details of FIG. 14 demonstrate that glycomacropeptide present in the cooled protein solution 40 was also present in the microfiltration retentate 44 and then the ultrafiltration retentate 54, and consequently also in the aqueous solution of the GMP powder. The large peak to the right of the GMP peak in the sample trace of FIG. 14 is a TCA artifact related to residuals of the TCA used to precipitate any β-lactoglobulin (and any α-lactalbumin) present in the sample, prior to analysis for glycomacropeptide at the 214 nanometer detection wavelength.

### Example 4

This Example demonstrates that the starting dairy material of Example 3, namely the 80% whey protein concentrate that was substituted in place of the ultrafiltration retentate 22 in the schematic drawing of FIG. 1, actually contained other κ-casein macropeptides in addition to glycomacropeptide.

Consequently, based upon the results of this Example, the GMP powder of Example 3 that was demonstrated to be rich in glycomacropeptide in Example 3 additionally constitutes κ-casein macropeptide-enriched powder that is rich in κ-casein macropeptide, including, but are not limited to, glycomacropeptide. Thus, in Example 3, where glycomacropeptide recovery was maximized in the microfiltration permeate 46, κ-casein macropeptides recovery was also maximized in the microfiltration permeate 46.

In this Example, Protein Analysis Procedure No. 2 that is described in the Property Determination and Characterization Techniques was employed to determine the concentrations of κ-casein macropeptide, β-lactoglobulin, and α-lactalbumin in the 80% whey protein concentrate (80% WPC) that was used as the starting material in Example 3 and in the GMP powder that was produced in Example 3. The GMP powder of Example 3 was formed by spray drying the ultrafiltration retentate 54 of Example 3.

A first aqueous solution of the GMP powder of Example 3, with a concentration of about 0.5 weight percent GMP powder based upon the total weight of the first aqueous solution, was produced by blending 0.126 grams of the GMP powder with 25 milliliters of purified water. Next, a second aqueous solution of the 80% WPC used as the starting material in Example 3, with a concentration of about 0.5 weight percent of the 80% WPC based upon the total weight of the second aqueous solution, was produced by blending 0.1251 grams of the 80% WPC with 25 milliliters of purified water. The purified water used to prepare both the first aqueous solution and the second aqueous solution had a resistance of about 18 Mega-ohms (about 0.06 μS/cm). The first aqueous solution of the GMP powder and the second aqueous solution of the 80% WPC were gently stirred for about two hours and were then centrifuged at a gravitational force of about 13,000 times the force of gravity (at sea level) for about ten minutes to remove any undissolved particulate.

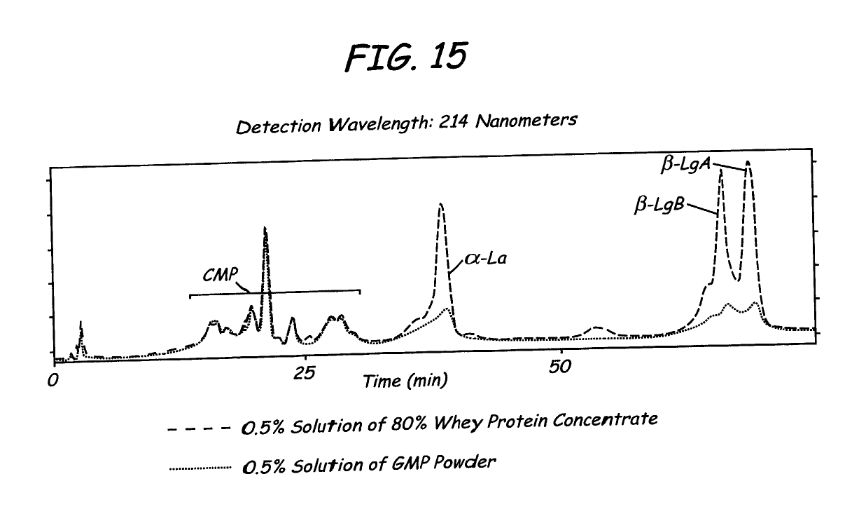

The first aqueous solution of the GMP powder and the second aqueous solution of the 80% WPC were then analyzed using the reversed phase HPLC technique of Protein Analysis Procedure No. 2 that is described in the Property Determination and Characterization Techniques to determine the concentrations of κ-casein macropeptide, β-lactoglobulin, and α-lactalbumin in the first aqueous solution and the second aqueous solution. The results of these reversed phase HPLC analyses are presented in FIG. 15.

The scans of FIG. 15 illustrate that the leftmost portions of the scans for the first aqueous solution and the second aqueous solution overlay and match each other almost identically. Thus, the scans of FIG. 15 demonstrate that the κ-casein macropeptide profile of the GMP powder produced in Example 3 is virtually identical to the κ-casein macropeptide profile of the 80% whey protein concentrate (80% WPC) that was used as the starting material in Example 3. Thus, the alkaline heating conditions employed in Example 3 in accordance with the present invention caused little if any modification or destruction of the κ-casein macropeptide originally present in the 80% whey protein concentrate. On the other hand, the scans of FIG. 15 illustrate that, consistent with the discussion in Example 3, the concentrations (peaks) of β-lactoglobulin and α-lactalbumin in the GMP powder of Example 3 are significantly diminished, as compared to the concentrations (peaks) of β-lactoglobulin and α-lactalbumin in the 80% whey protein concentrate used as the starting material in Example 3.

Next, a third aqueous solution of the volumetric standard for glycomacropeptide (obtained from Sigma Chemical Company of St. Louis, Mo.) used in Protein Analysis Procedure No. 1 was prepared, with a concentration of about 0.5 weight percent of the glycomacropeptide volumetric standard based upon the total weight of the third aqueous solution, was produced by blending 0.01 grams of the glycomacropeptide volumetric standard with two milliliters of purified water. The purified water used to prepare the third aqueous solution had a resistance of about 18 Mega-ohms (about 0.06 μS/cm). The third aqueous solution of the glycomacropeptide volumetric standard was gently stirred for about two hours and was then centrifuged at a gravitational force of about 13,000 times the force of gravity (at sea level) for about ten minutes to remove any undissolved particulate.

The third aqueous solution of the glycomacropeptide volumetric standard was then analyzed using the reversed phase HPLC technique of Protein Analysis Procedure No. 2 that is described in the Property Determination and Characterization Techniques to determine the concentrations of κ-casein macropeptide, β-lactoglobulin, and α-lactalbumin in the third aqueous solution.

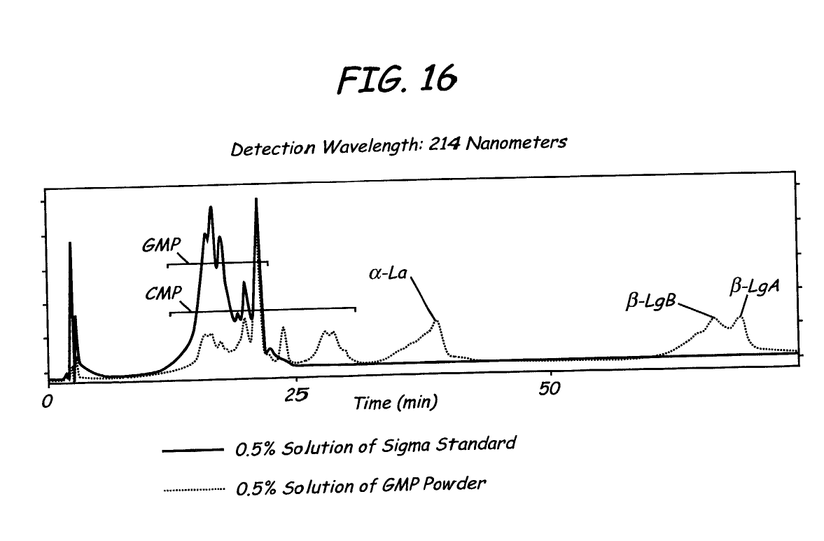

The results of this reversed phase HPLC analysis for the third aqueous solution of the glycomacropeptide volumetric standard and the previously discussed results of the reversed phase HPLC analysis for the first aqueous solution of the GMP powder of Example 3 are presented in FIG. 16.

First, the scans of FIG. 16 illustrate that, as would be expected, the Sigma glycomacropeptide volumetric standard is free, or essentially free, of β-lactoglobulin and α-lactalbumin since the FIG. 16 scan of the solution of the Sigma glycomacropeptide volumetric standard exhibits no peaks for β-lactoglobulin or α-lactalbumin. On the other hand, as explained with reference to FIG. 15, the FIG. 16 scan for the solution of the GMP powder produced in Example 3 does exhibit peaks for both β-lactoglobulin and α-lactalbumin.

Next, the scans of FIG. 16 illustrate that the leftmost portions of the scans for the first aqueous solution and the third aqueous solution do not overlay or match each other very well at all. Thus, the scans of FIG. 15 demonstrate that the macropeptide profile of the GMP powder produced in Example 3 is quite a bit different from the glycomacropeptide peptide profile of the Sigma glycomacropeptide volumetric standard used in Protein Analysis Procedure No. 1. Indeed, the range of retention times for the macropeptide peaks of the GMP powder is broader than the range of retention times for the glycomacropeptide volumetric standard itself in FIG. 16. This demonstrates that the GMP powder contains other κ-casein macropeptides in addition to glycomacropeptide. Indeed, as depicted in FIG. 15, the κ-casein macropeptide profile of the GMP powder produced in Example 3 is virtually identical to the κ-casein macropeptide profile of the 80% whey protein concentrate (80% WPC) that was used as the starting material in Example 3. Therefore, the GMP powder of Example 3 that was demonstrated to be rich in glycomacropeptide in Example 3 additionally constitutes κ-casein macropeptide-enriched powder that is rich in κ-casein macropeptide, including, but are not limited to, glycomacropeptide.

### Example 5

This Example demonstrates processing of a starting dairy material that contains glycomacropeptide, α-lactalbumin, and β-lactoglobulin to produce a polymerized protein powder that is rich in polymers of α-lactalbumin and β-lactoglobulin and a GMP powder that is rich in glycomacropeptide. One goal of this Example was to maximize conversion of β-lactoglobulin and α-lactalbumin to polymers of β-lactoglobulin and α-lactalbumin, as opposed to either maximizing preservation of glycomacropeptide or maximizing recovery of glycomacropeptide in the microfiltration permeate 46.

The details of this Example, except as otherwise described herein, were identical to the process details of Example 3, with two exceptions. In this Example, the alkaline intermediate 32, upon passage through the high temperature short time (HTST) plate heat exchanger, had a temperature of about 213° F. in this Example, rather than the temperature of about 177° F. of the alkaline intermediate 32, upon passage through the high temperature short time (HTST) plate heat exchanger, in Example 3. Also, in this Example, 100 gallons of the combined microfiltration permeate 46 was obtained. The combined microfiltration permeate 46 of this Example, like the combined microfiltration permeate 46 of Example 3, was composed of both (1) the microfiltration permeate 46 that was obtained during the initial microfiltration of the cooled protein solution 40 and (2) the microfiltration permeate 46 obtained during diafiltration of the microfiltration retentate 44.

Details about the component weights for the 80% whey protein concentrate (which served as the ultrafiltration retentate 22 in this Example); the intermediates 28, 32; the cooled protein solution 20; the microfiltration retentate 44; the microfiltration permeate 46; the polymerized protein powder 50; and the glycomacropeptide powder that was derived by spray drying the acidified ultrafiltration retentate 54 are provided in Table 13 below:

TABLE 13

| | Total | Total | Total | Total | | | |
|---|---|---|---|---|---|---|---|
| | Solids | Protein | Fat | Ash | β-lg* | α-la** | GMP*** |
| Stream Description | (lbs.) | (lbs.) | (lbs) | (lbs.) | (lbs.) | (lbs.) | (lbs.) |
| Alkaline (pH˜7.7) Intermediate 32 | 34.83 | 28.06 | | | 16.48 | 4.42 | 3.04 |
| Cooled Protein Solution 40 | 34.57 | 27.61 | 2.13 | 1.29 | 2.18 | 0.84 | 3.03 |
| Microfiltration Retentate 44 | 19.45 | 16.67 | 2.41 | 0.33 | 0.46 | 0.18 | 0.20 |
| Microfiltration Permeate 46 | 15.89 | 11.64 | <0.09 | 0.95 | 2.31 | 0.57 | 2.34 |
| Polymerized Protein Powder 50 | 11.69 | 10.01 | 1.47 | 0.20 | | | |
| Glycomacropeptide Powder | 8.23 | 7.32 | 0.04 | 0.37 | | | |

*β-lactoglobulin
**α-lactalbumin
***glycomacropeptide

The details of Table 13 demonstrate that the polymerized protein powder 50 is, on a weight basis, about 82% protein (as total protein), about 12% fat, and about 1.6% ash, based upon the total weight of the polymerized protein powder 50. These details of Table 13 also demonstrate that the glycomacropeptide powder is, on a weight basis, about 84% protein (as total protein), about 0.4% fat, and about 4.2% ash, based upon the total weight of the glycomacropeptide powder.

The total solids, total protein, total fat, and total ash weights presented in Table 13 were calculated, using the total weight of the particular stream, after determining the weight percent total solids, weight percent total protein, weight percent total fat, and weight percent total ash of the different streams using the procedures for these particular determinations that are described in the Property Determination and Characterization Techniques section of this document. Also, the β-lactoglobulin, α-lactalbumin, and glycomacropeptide weights presented in Table 13 were calculated, using the total volume of the particular stream, after determining the weight-based concentration (mg/ml) of β-lactoglobulin, α-lactalbumin, and glycomacropeptide of the different streams using Protein Analysis Procedure No. 1 that is described in the Property Determination and Characterization Techniques section of this document, with the exception that a pair of 300 mm×7.8 mm Bio-Sil SEC 125 size exclusion columns were joined in series and substituted for the single 300 mm×7.8 mm Bio-Sil SEC 125 size exclusion column prescribed in Protein Analysis Procedure No. 1.

Minor discrepancies between the total solids and the total protein present in the combination of the microfiltration retentate 44 and the microfiltration permeate 46 versus the total solids and the total protein in the intermediates 28, 32, and the cooled protein solution 40, respectively, is believed due, in large part at least, to residual amounts of insoluble proteins that remained following transfer of the cooled protein solution 40 to the microfilter 42. The decreasing ratios of the combined weighs of β-lactoglobulin, α-lactalbumin, and glycomacropeptide for a particular stream to the total protein weight for the particular stream, when going from the intermediates 28, 32, to the cooled protein solution 40, to the microfiltration retentate 44 and to the microfiltration permeate 46 reflects the desired loss of solubility of β-lactoglobulin and α-lactalbumin, upon heating of the intermediate 32. Also, as seen in Table 13, the split between the total protein content of the microfiltration retentate 44 and the total protein content of the microfiltration permeate 46 is more pronounced than in Example 3, since the focus of this Example is to maximize conversion of β-lactoglobulin and α-lactalbumin to polymers of β-lactoglobulin and α-lactalbumin, as opposed to maximizing preservation of glycomacropeptide and maximizing recovery of glycomacropeptide in the microfiltration permeate 46.

Next, percent recovery data for total solids, total protein, total fat, total ash, β-lactoglobulin, α-lactalbumin, and glycomacropeptide ranging from the intermediates 30, 32, to the cooled protein solution 40, to the microfiltration retentate 44 and the microfiltration permeate 46, and ending with the polymerized protein powder 50 and the glycomacropeptide powder that was derived by spray drying the acidified ultrafiltration retentate 54 were calculated from the weight data of Table 13 and are provided in Table 14 below:

TABLE 14

| | Total | Total | Total | | | | |
|---|---|---|---|---|---|---|---|
| | Solids | Protein | Fat | Total Ash | β-lg* | α-la** | GMP*** |
| Stream Description | (% Rec.)^{A} | (% Rec.)^{A} | (% Rec.)^{A} | (% Rec.)^{A} | (% Rec.)^{A} | (% Rec.)^{A} | (% Rec.)^{A} |
| Cooled Protein Solution 40 | 99 | 98 | | | 13 | 19 | 100 |
| Microfiltration Retentate 44 | 56 | 60 | 113 | 25 | 3 | 4 | 7 |
| Microfiltration Permeate 46 | 46 | 42 | <4 | 73 | 14 | 13 | 77 |
| Polymerized Protein Powder 50 | 34 | 36 | 69 | 16 | | | |
| Glycomacropeptide Powder | 24 | 27 | 2 | 28 | | | |

*β-lactoglobulin
**α-lactalbumin
***glycomacropeptide
^{A}Percent recovery of the particular component in a particular stream versus the amount of the component orginally present in the diluted 80% WPC (as UF 1 retentate 22).

The data of Table 14 demonstrate that only about 84% of the glycomacropeptide originally present in the diluted 80% WPC remained, following heating as the heated intermediate 36, soluble in the microfiltration retentate 44 and the microfiltration permeate 46, collectively. Also, only 14% of the β-lactoglobulin and 13% of the α-lactalbumin remained soluble in the cooled protein solution 40 following the described heating. These observations are consistent with the focus of this Example to maximize conversion of β-lactoglobulin and α-lactalbumin to polymers of β-lactoglobulin and α-lactalbumin, as opposed to maximizing preservation of glycomacropeptide and maximizing recovery of glycomacropeptide in the microfiltration permeate 46.

Next, the concentrations of β-lactoglobulin, α-lactalbumin, glycomacropeptide, Immunoglobulin G, and bovine serum albumin were determined using Protein Analysis Procedure No. 1 that is described in the Property Determination and Characterization Techniques section of this document for an aqueous solution of the glycomacropeptide powder derived by spray drying the acidified ultrafiltration retentate 54. In this HPLC determination, the single 300 mm×7.8 mm Bio-Sil SEC 125 size exclusion column prescribed in Protein Analysis Procedure No. 1 was used. The aqueous solution that was analyzed was prepared by blending 510.2 milligrams of the glycomacropeptide powder with 100 milliliters of water to yield the aqueous solution with a concentration of 5.10 milligrams of glycomacropeptide per milliliter of water.

The Total Protein determination procedure from the Property Determination and Characterization Techniques section of this document was employed to determine that the glycomacropeptide powder of this Example was about 88.41 weight percent protein (as total protein), based upon the total weight of the glycomacropeptide powder. The concentrations of β-lactoglobulin, α-lactalbumin, glycomacropeptide, Immunoglobulin G, and bovine serum albumin in the aqueous solution of the glycomacropeptide powder were 0.20, 0.50, 2.11, 0, and 0 milligrams per milliliter (mg/ml) of the aqueous solution, respectively. Using these concentration details and the 88.41 weight percent protein (as total protein) known to be in the glycomacropeptide powder, the weight percentages of β-lactoglobulin, α-lactalbumin, and glycomacropeptide relative to the total weight of the glycomacropeptide powder (as-is basis) and the weight percentages of β-lactoglobulin, α-lactalbumin, and glycomacropeptide relative to each other (protein basis) were determined and are presented in Table 15 below:

TABLE 15

| | β-lg* | α-la** | GMP*** |
|---|---|---|---|
| Stream Description | (weight %) | (weight %) | (weight %) |
| glycomacropeptide powder | 4 | 10 | 41 |
| (“as is” basis^{A}) | | | |
| glycomacropeptide powder | 4 | 11 | 47 |
| (“protein” basis^{B}) | | | |

*β-lactoglobulin
**α-lactalbumin
***glycomacropeptide
^{A}as a percentage of the total weight of the glycomacropeptide powder
^{B}as a percent of the total weight of β-lactoglobulin, α-lactalbumin, and glycomacropeptide present in the glycomacropeptide powder

Thus, the data of Table 15 demonstrate that the glycomacropeptide powder produced in this Example was a little more than about 40 weight percent glycomacropeptide, based upon the total weight of the glycomacropeptide powder. Additional, this data demonstrate that slightly less than about one half of the protein in the glycomacropeptide powder of this Example was glycomacropeptide, based upon the weight of the glycomacropeptide and the total weight of the glycomacropeptide powder.

Furthermore, the Table 15 data show that glycomacropeptide, β-lactoglobulin, and α-lactalbumin constitute only about 62% of the protein present in the GMP powder, while aggregated protein molecules (i.e.: polymers of β-lactoglobulin and α-lactalbumin) constitute most, if not all, of the remaining protein present in the GMP powder. On the other hand, as seen from the Table 11 data, glycomacropeptide, β-lactoglobulin, and α-lactalbumin constituted about 100% of the protein present in the GMP powder of Example 3, while aggregated protein molecules (i.e.: polymers of β-lactoglobulin and α-lactalbumin) constituted little, if any, of the protein present in the GMP powder of Example 3.

Next, Table 16 provides a comparison of the weight percentages of β-lactoglobulin, α-lactalbumin, and glycomacropeptide relative to each other (protein basis) in both the starting 80% whey protein concentrate that was diluted with water to form the feed (ultrafiltration retentate 22) of this Example and in the glycomacropeptide powder produced in this Example:

TABLE 16

| | β-lg* | α-la** | GMP*** |
|---|---|---|---|
| Stream Description | (weight %) | (weight %) | (weight %) |
| 80% Whey Protein | 62 | 17 | 11 |
| Concentrate (“protein” basis^{A}) | | | |
| Glycomacropeptide Powder | 4 | 11 | 47 |
| (“protein” basis^{B}) | | | |

*β-lactoglobulin
**α-lactalbumin
***glycomacropeptide
^{A}as a percent of the total weight of β-lactoglobulin, α-lactalbumin, and glycomacropeptide present in the starting 80% whey protein concentrate
^{B}as a percentage of the total weight of β-lactoglobulin, α-lactalbumin, and glycomacropeptide present in the glycomacropeptide powder

Thus, the data of Table 16 show the concentration of glycomacropeptide in the glycomacropeptide powder was about 4.10 times greater (47÷11) than the concentration of glycomacropeptide in the starting 80% whey protein concentrate that was diluted with water to form the feed (ultrafiltration retentate 22) of this Example.

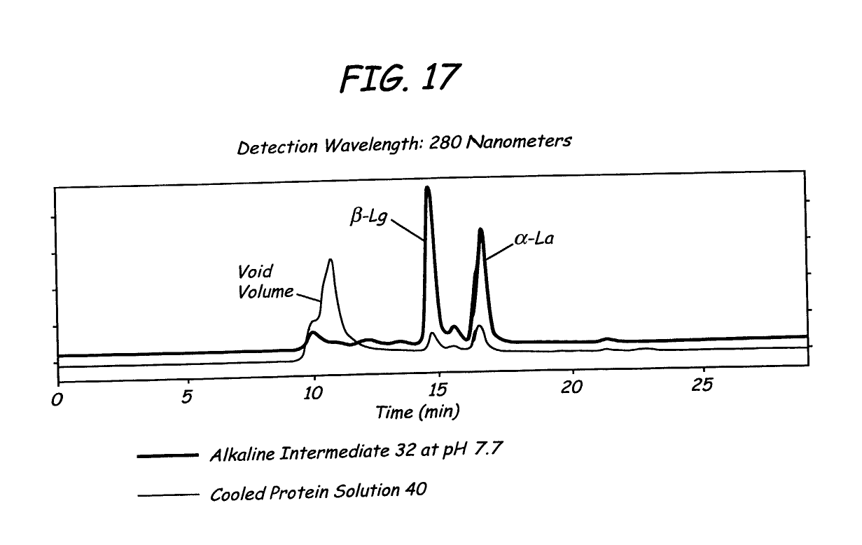

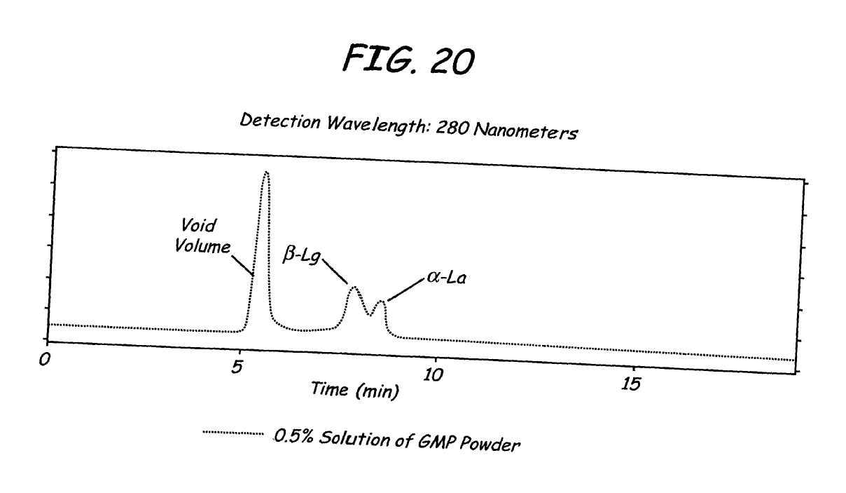

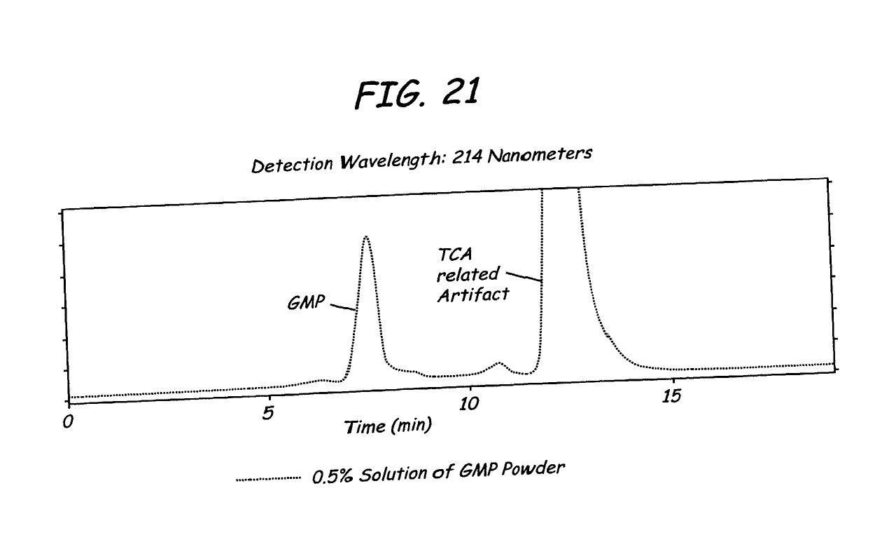

Samples of various streams described above in this Example were subjected to HPLC analysis based upon size-exclusion in accordance with Protein Analysis Procedure No. 1 that is provided above in the Property Determination And Characterization Techniques section of this document. In the HPLC procedure, when producing the scans of FIGS. 17-19, a pair of 300 mm×7.8 mm Bio-Sil SEC 125 size exclusion columns were joined in series and substituted for the single 300 mm×7.8 mm Bio-Sil SEC 125 size exclusion column prescribed in Protein Analysis Procedure No. 1. On the other hand, when producing the scans of FIGS. 20-21, the stationary phase of the chromatographic system was the single 300 mm×7.8 mm Bio-Sil SEC 125 size exclusion column, as specified in Protein Analysis Procedure No. 1, rather than the pair of 300 mm×7.8 mm Bio-Sil SEC 125 size exclusion columns, joined in series, that were used when producing the scans of FIGS. 17-19.

As depicted in FIG. 17, the concentrations of both α-lactalbumin and β-lactoglobulin were decreased substantially in the cooled protein solution 40 of this Example versus the concentrations of both α-lactalbumin and β-lactoglobulin in the alkaline intermediate 32 (5% aqueous solution of 80% whey protein concentrate at pH 7.7) of this Example. Furthermore, from FIG. 17, it is clear that the relative concentration of α-lactalbumin is decreased less from the alkaline intermediate 32 to the cooled protein solution 40, as compared to the relative concentration decrease of β-lactoglobulin from the alkaline intermediate 32 to the cooled protein solution 40. On the other hand, in FIG. 17, the void volume peak, which represents polymerized protein, is relatively large for the cooled protein solution 40, while the void volume peak is relatively small for the alkaline intermediate 32.

Consistent with the discussion in Examples 1 and 3 above, the large void volume peak in FIG. 17 for the cooled protein solution 40 represents molecules with a molecular weight on the order of about 200,000 Daltons, namely polymers of α-lactalbumin and β-lactoglobulin that are formed by sulfur bridging between cysteine residues of the original α-lactalbumin and β-lactoglobulin. The fact that the polymers of α-lactalbumin and β-lactoglobulin represented by the large void volume peak are observed in the scan at all indicates these polymers of α-lactalbumin and β-lactoglobulin, by virtue of entering the HPLC column, are soluble in the aqueous solution being analyzed by the HPLC procedure.

Here, FIG. 17 demonstrates that, after heating of the alkaline intermediate 32 in accordance with the heating details of this Example, polymers of α-lactalbumin and β-lactoglobulin are present in the cooled protein solution 40, these polymers of α-lactalbumin and β-lactoglobulin are soluble in the cooled protein solution 40 (as evidenced by the presence of the large void volume peak), and, consequently, less α-lactalbumin and less β-lactoglobulin remains in the cooled protein solution 40 than was originally present in the alkaline intermediate 32. The enhanced reduction of α-lactalbumin and β-lactoglobulin between the alkaline intermediate 32 and the cooled protein solution 40 in this Example versus the reduction experienced in Example 3 is believed due to the different heating approach taken in this Example versus the heating approach taken in Example 3.

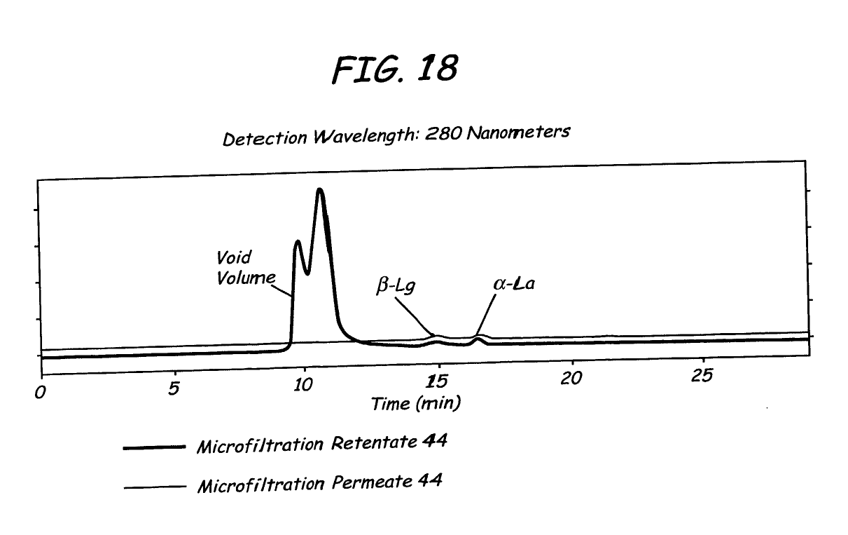

In FIG. 18, details about the microfiltration retentate 44 and the microfiltration permeate 46 are shown. The sample of the microfiltration retentate 44 that was analyzed for FIG. 18 was obtained from the diafiltered microfiltration retentate 44 remaining after both (1) the initial microfiltration of the cooled protein solution 40 and (2) the three stage diafiltration of the initial microfiltration retentate 44. The sample of the microfiltration permeate 46 that was analyzed for FIG. 18 was obtained from the 100 gallon combined microfiltration permeate 46 stream that included both (1) the microfiltration permeate 46 obtained during the initial microfiltration of the cooled protein solution 40 and (2) the microfiltration permeate 46 obtained during the three stage diafiltration of the microfiltration retentate 44.

These details of FIG. 18 show that very little α-lactalbumin or β-lactoglobulin is present in the microfiltration retentate 44, while slightly higher concentrations of α-lactalbumin and β-lactoglobulin are present in the microfiltration retentate 44 as compared to the cooled protein solution 40. However here, as compared to the somewhat larger concentrations of α-lactalbumin and β-lactoglobulin remaining in the microfiltration retentate 44 of Example 3, the significantly smaller concentrations of α-lactalbumin and β-lactoglobulin remaining in the microfiltration retentate 44 of this Example demonstrate that the different heating approach taken in this Example, versus the heating approach taken in Example 3, caused polymerization of a higher percentage of the α-lactalbumin and β-lactoglobulin originally present in the alkaline intermediate 32 of this Example, as compared to the percentage of α-lactalbumin and β-lactoglobulin polymerized in Example 3. This observation is consistent with the focus of this Example to maximize conversion of β-lactoglobulin and α-lactalbumin to polymers of β-lactoglobulin and α-lactalbumin, as opposed to maximizing preservation of glycomacropeptide and maximizing recovery of glycomacropeptide in the microfiltration permeate 46.

In FIG. 18, a pair of adjoining void volume peaks exist for the microfiltration retentate 44. The right hand void volume is significantly larger than the left hand void volume peak of the microfiltration retentate 44. On the other hand, virtually no void volume peak is visible in FIG. 18 for the microfiltration permeate 46. Consistent with the previous discussion in this Example regarding FIG. 17 and the previous discussion in Examples 1 and 3 above, the large right hand void volume peak in FIG. 18 for the microfiltration retentate 44 represents polymers of α-lactalbumin and β-lactoglobulin with a molecular weight on the order of about 200,000 Daltons. Correspondingly, the somewhat smaller left hand void volume peak in FIG. 18 for the microfiltration retentate 44 represents polymers of α-lactalbumin and β-lactoglobulin with a molecular weight somewhat larger than about 200,000 Daltons.

The relatively large void volume peaks for the microfiltration retentate 44 sample demonstrates that (1) a substantial concentration of polymers of α-lactalbumin and β-lactoglobulin is present in the microfiltration retentate 44 and (2) these polymers of α-lactalbumin and β-lactoglobulin are soluble in the cooled protein solution 40 (as evidenced by the presence of the relatively large void volume peak); consequently, as evidenced by the virtually non-existent α-lactalbumin and β-lactoglobulin peaks in FIG. 18, very little α-lactalbumin and very little β-lactoglobulin remains in the microfiltration retentate 44. Correspondingly, the virtually non-existent void volume peak for the microfiltration permeate 46 sample demonstrates that few, if any, polymers of α-lactalbumin and β-lactoglobulin are present in the microfiltration permeate 46.

Thus, FIG. 18 illustrates that the microfiltration excluded the polymerized proteins (polymers of α-lactalbumin and β-lactoglobulin), since the polymerized protein concentration of the microfiltration retentate 44 is much larger than the essentially non-existent polymerized protein concentration of the microfiltration permeate 46, as evidenced by the much larger void volume peaks of the microfiltration retentate 44 as compared to the virtually non-existent void volume peak of the microfiltration permeate 46. Furthermore, FIG. 18 illustrates that very little non-polymerized α-lactalbumin and very little non-polymerized β-lactoglobulin is present in the microfiltration retentate 44, while only a relatively small concentration of both non-polymerized α-lactalbumin and non-polymerized β-lactoglobulin is present in the microfiltration permeate 46.

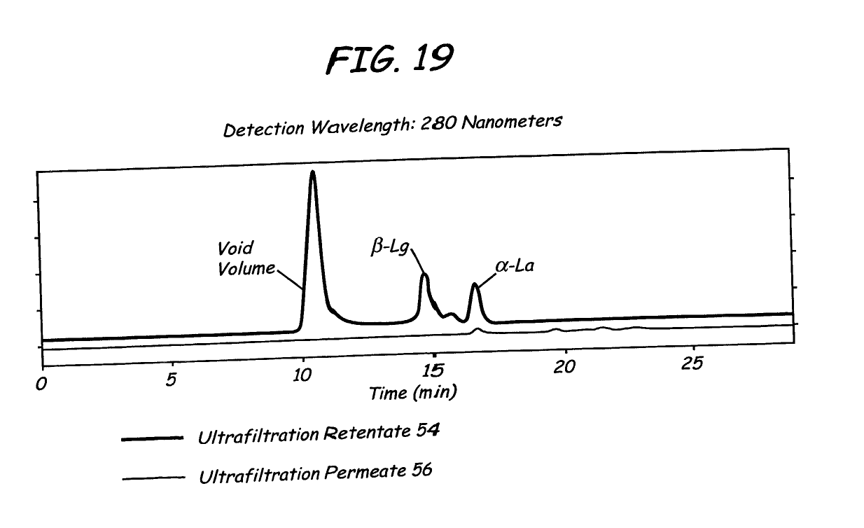

Details about the α-lactalbumin and β-lactoglobulin contents of the ultrafiltration retentate 54 and the ultrafiltration permeate 56 are provided in FIG. 19. The ultrafiltration retentate 54 and the ultrafiltration permeate 56 that were analyzed for FIG. 19 were each obtained from ultrafiltration of the 110 gallon combined microfiltration permeate 46 stream that included both (1) the microfiltration permeate 46 obtained during the initial microfiltration of the cooled protein solution 40 and (2) the microfiltration permeate 46 obtained during the three stage diafiltration of the microfiltration retentate 44.

As noted above, with reference to FIG. 18, very little non-polymerized α-lactalbumin and very little non-polymerized β-lactoglobulin was present in the microfiltration permeate 46, and no polymerized proteins (polymers of α-lactalbumin and β-lactoglobulin) were present in the microfiltration permeate 46. FIG. 19 demonstrates that the vast majority of the non-polymerized α-lactalbumin and all of the non-polymerized β-lactoglobulin present in the microfiltration permeate 46 addressed in FIG. 18 remained in the ultrafiltration retentate 54. Correspondingly, FIG. 19 illustrates that only a very minimal amount of the non-polymerized α-lactalbumin and none of the non-polymerized 6 -lactoglobulin present in the microfiltration permeate 46 addressed in FIG. 18 passed through the ultrafiltration membrane as part of the ultrafiltration permeate 56.

Also, the presence of the significant void volume peak for the ultrafiltration retentate 54 sample demonstrates that (1) a significant concentration of polymers of α-lactalbumin and β-lactoglobulin exists in the ultrafiltration retentate 54 and (2) these polymers of α-lactalbumin and β-lactoglobulin are soluble in the ultrafiltration retentate 54 (as evidenced by the presence of the significant void volume peak). The significant concentration of polymers of α-lactalbumin and β-lactoglobulin in the ultrafiltration retentate 54 is interesting, considering that FIG. 18 showed that very little, if any, polymers of α-lactalbumin and β-lactoglobulin were present in the microfiltration permeate 46 that was ultrafiltered to form the ultrafiltration retentate. As explained in Example 3 above, it is thought that the polymers of α-lactalbumin and β-lactoglobulin found in the ultrafiltration retentate 54 may be formed during ultrafiltration by oxidation that catalyzes a sulfur bridging reaction between different cysteine residues (i.e. cysteine sulfhydryl groups) of α-lactalbumin and β-lactoglobulin present in the microfiltration permeate 46.

In FIG. 20, details about a 0.5 weight percent aqueous solution of the GMP powder, based upon the total weight of the solution, are shown. The GMP powder was formed by spray drying the ultrafiltration retentate 54. These details of FIG. 20 demonstrate that some non-polymerized α-lactalbumin and some non-polymerized β-lactoglobulin is present in the aqueous GMP powder solution. Also, the presence of the significant void volume peak for the ultrafiltration retentate 54 sample demonstrates that (1) polymers of α-lactalbumin and β-lactoglobulin are present in the aqueous GMP powder solution and (2) these polymers of α-lactalbumin and β-lactoglobulin are soluble in the aqueous GMP powder solution (as evidenced by the presence of the significant void volume peak).

Furthermore, the peaks for the non-polymerized α-lactalbumin, non-polymerized β-lactoglobulin, and the void volume peak of the aqueous GMP powder solution have about the same amplitude as the peaks for the non-polymerized α-lactalbumin, non-polymerized β-lactoglobulin, and the void volume peak of the ultrafiltration retentate 54. The void volume peak data of FIG. 20 also substantiate the aforementioned conclusion about the Table 15 data showing that aggregated protein molecules (i.e.: polymers of β-lactoglobulin and α-lactalbumin) constitute most, if not all, of the 32% of the protein present in the GMP powder not present as glycomacropeptide, β-lactoglobulin, and/or α-lactalbumin. Specifically, the void volume peak in FIG. 20 is significantly taller than the corresponding void volume peak in FIG. 13 of Example 3.

Also, the amplitude of the void volume peak in FIG. 20 relative to the amplitude of the β-lactoglobulin peak and the CL-lactalbumin peak in FIG. 20 is significantly greater than amplitude of the corresponding void volume peak in FIG. 13 of Example 3 relative to the amplitude of the β-lactoglobulin peak and the α-lactalbumin peak in FIG. 13 of Example 3. These two observations about the void volume peak of FIG. 20, relative to the void volume peak of FIG. 13 of Example 3, support and substantiate the conclusion about the Table 15 data of this Example showing that aggregated protein molecules (i.e.: polymers of β-lactoglobulin and α-lactalbumin) constitute most, if not all, of the 32% of the protein present in the GMP powder not present as glycomacropeptide, β-lactoglobulin, and/or α-lactalbumin versus the conclusion about the Table 11 data showing that aggregated protein molecules (i.e.: polymers of β-lactoglobulin and α-lactalbumin) constitute little, if any, of the protein present in the GMP powder of Example 3.

It is noted that the peaks in FIG. 20 for the non-polymerized α-lactalbumin and non-polymerized β-lactoglobulin of the aqueous GMP powder solution have somewhat different shapes adjacent to each other, as compared to the peaks for the non-polymerized α-lactalbumin and non-polymerized β-lactoglobulin of the ultrafiltration retentate 54. This apparent difference in shape is believed due solely to the use of a single Bio-Sil SEC 125 size exclusion column for the HPLC analysis of the aqueous GMP powder solution, as compared to the use of two serially-joined Bio-Sil SEC 125 size exclusion columns for the HPLC analysis of the ultrafiltration retentate 54, which would be expected to reduce the resolution of the HPLC trace for the aqueous GMP powder solution versus the resolution of the HPLC trace for the ultrafiltration retentate 54.

In FIG. 21, additional details about the 0.5 weight percent aqueous solution of the GMP powder are shown. These details of FIG. 21 demonstrate that glycomacropeptide present in the cooled protein solution 40 was also present in the microfiltration retentate 44 and then the ultrafiltration retentate 54, and consequently also in the aqueous solution of the GMP powder. The large peak to the right of the GMP peak in the sample trace of FIG. 21 is a TCA artifact related to residuals of the TCA used to precipitate any β-lactoglobulin (and any α-lactalbumin), prior to analysis for glycomacropeptide at the 214 nanometer detection wavelength.

### Example 6

This Example demonstrates that the starting dairy material of Example 5, namely the 80% whey protein concentrate that was substituted in place of the ultrafiltration retentate 22 in the schematic drawing of FIG. 1, actually contained other κ-casein macropeptide in addition to glycomacropeptide. Consequently, based upon the results of this Example, the GMP powder of Example 5 that was demonstrated to be rich in glycomacropeptide in Example 5 additionally constitutes κ-casein macropeptide-enriched powder that is rich in κ-casein macropeptide, including, but are not limited to, glycomacropeptide. Thus, in Example 5, where glycomacropeptide recovery was maximized in the microfiltration permeate 46, κ-casein macropeptides recovery was also maximized in the microfiltration permeate 46.

In this Example, Protein Analysis Procedure No. 2 that is described in the Property Determination and Characterization Techniques was employed to determine the concentrations of κ-casein macropeptide, β-lactoglobulin, and α-lactalbumin in the 80% whey protein concentrate (80% WPC) that was used as the 110 starting material in Example 5 and in the GMP powder that was produced in Example 5. The GMP powder of Example 4 was formed by spray drying the ultrafiltration retentate 54 of Example 5.

A first aqueous solution of the GMP powder of Example 5, with a concentration of about 0.5 weight percent GMP powder based upon the total weight of the first aqueous solution, was produced by blending 0.1257 grams of the GMP powder with 25 milliliters of purified water. Next, a second aqueous solution of the 80% WPC used as the starting material in Example 5, with a concentration of about 0.5 weight percent of the 80% WPC based upon the total weight of the second aqueous solution, was produced by blending 0.1251 grams of the 80% WPC with 25 milliliters of purified water. The purified water used to prepare both the first aqueous solution and the second aqueous solution had a resistance of about 18 Mega-ohms (about 0.06 μS/cm). The first aqueous solution of the GMP powder and the second aqueous solution of the 80% WPC were gently stirred for about two hours and were then centrifuged at a gravitational force of about 13,000 times the force of gravity (at sea level) for about ten minutes to remove any undissolved particulate.

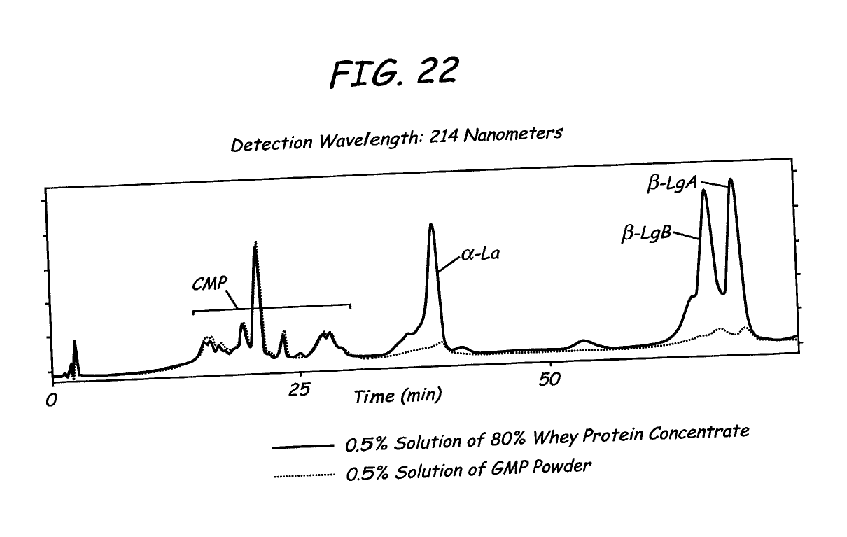

The first aqueous solution of the GMP powder and the second aqueous solution of the 80% WPC were then analyzed using the reversed phase HPLC technique of Protein Analysis Procedure No. 2 that is described in the Property Determination and Characterization Techniques to determine the concentrations of κ-casein macropeptide, β-lactoglobulin, and α-lactalbumin in the first aqueous solution and the second aqueous solution. The results of these reversed phase HPLC analyses are presented in FIG. 22.

The scans of FIG. 22 illustrate that the leftmost portions of the scans for the first aqueous solution and the second aqueous solution overlay and match each other almost identically. Thus, the scans of FIG. 22 demonstrate that the κ-casein macropeptide profile of the GMP powder produced in Example 5 is virtually identical to the κ-casein macropeptide profile of a production 80% whey protein concentrate (80% WPC) that was very similar to 80% WPC used as the starting material in Example 5. Thus, the alkaline heating conditions employed in Example 5 in accordance with the present invention caused little if any modification or destruction of the κ-casein macropeptide, as compared to the κ-casein macropeptide present in the production 80% whey protein concentrate. On the other hand, the scans of FIG. 22 illustrate that, consistent with the discussion in Example 5, the concentrations (peaks) of β-lactoglobulin and β-lactalbumin in the GMP powder of Example 5 are significantly diminished, as compared to the concentrations (peaks) of β-lactoglobulin and α-lactalbumin in the production 80% whey protein concentrate.

Next, a third aqueous solution of the volumetric standard for glycomacropeptide (obtained from Sigma Chemical Company of St. Louis, Mo.) used in Protein Analysis Procedure No. 1 was prepared, with a concentration of about 0.5 weight percent of the glycomacropeptide volumetric standard based upon the total weight of the third aqueous solution, was produced by blending 0.01 grams of the glycomacropeptide volumetric standard with two milliliters of purified water. The purified water used to prepare the third aqueous solution had a resistance of about 18 Mega-ohms (about 0.06 μS/cm). The third aqueous solution of the glycomacropeptide volumetric standard was gently stirred for about two hours and was then centrifuged at a gravitational force of about 13,000 times the force of gravity (at sea level) for about ten minutes to remove any undissolved particulate.

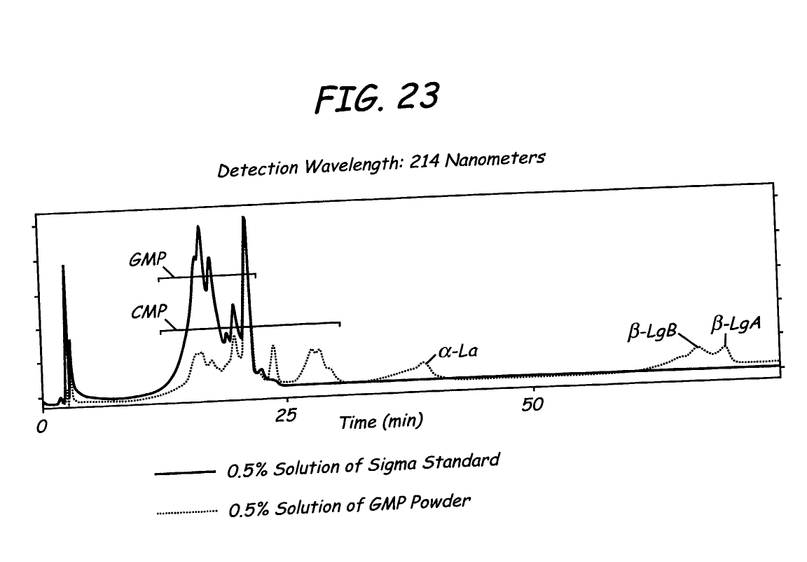

The third aqueous solution of the glycomacropeptide volumetric standard was then analyzed using the reversed phase HPLC technique of Protein Analysis Procedure No. 2 that is described in the Property Determination and Characterization Techniques to determine the concentrations of κ-casein macropeptide, β-lactoglobulin, and α-lactalbumin in the third aqueous solution. The results of this reversed phase HPLC analysis for the third aqueous solution of the glycomacropeptide volumetric standard and the previously discussed results of the reversed phase HPLC analysis for the first aqueous solution of the GMP powder of Example 5 are presented in FIG. 23.

First, the scans of FIG. 23 illustrate that, as would be expected, the Sigma glycomacropeptide volumetric standard is free, or essentially free, of β-lactoglobulin and α-lactalbumin since the FIG. 23 scan of the solution of the Sigma glycomacropeptide volumetric standard exhibits no peaks for β-lactoglobulin or α-lactalbumin. On the other hand, as explained with reference to FIG. 22, the FIG. 23 scan for the solution of the GMP powder produced in Example 5 does exhibit peaks for both β-lactoglobulin and α-lactalbumin.

Next, the scans of FIG. 23 illustrate that the leftmost portions of the scans for the first aqueous solution and the third aqueous solution do not overlay or match each other very well at all. Thus, the scans of FIG. 22 demonstrate that the macropeptide profile of the GMP powder produced in Example 5 is quite a bit different from the glycomacropeptide peptide profile of the Sigma glycomacropeptide volumetric standard used in Protein Analysis Procedure No. 1. Indeed, the range of retention times for the macropeptide peaks of the GMP powder is broader than the range of retention times for the glycomacropeptide volumetric standard itself in FIG. 23. This demonstrates that the GMP powder contains other κ-casein macropeptides in addition to glycomacropeptide. Indeed, as depicted in FIG. 22, the κ-casein macropeptide profile of the GMP powder produced in Example 5 is virtually identical to the κ-casein macropeptide profile of the 80% whey protein concentrate (80% WPC) that was used as the starting material in Example 5. Therefore, the GMP powder of Example 5 that was demonstrated to be rich in glycomacropeptide in Example 5 additionally constitutes κ-casein macropeptide-enriched powder that is rich in κ-casein macropeptide, including, but are not limited to, glycomacropeptide.

Although the present invention has been described with reference to preferred embodiments, workers skilled in the art will recognize that changes may be made in form and detail without departing from the spirit and scope of the invention.

claim(s):

1. A method of fractionating a first proteinaceous material or a second proteinaceous material, the method comprising:
heating the first proteinaceous material to an elevated temperature to yield a third proteinaceous material, the first proteinaceous material comprising κ-casein macropeptide and a heat labile protein, the pH of the first proteinaceous material being greater than about 7, and the elevated temperature effective to polymerize at least some of the heat labile protein present in the first proteinaceous material; or
providing the second proteinaceous material with a pH greater than about 7 to yield the third proteinaceous material, the second proteinaceous material comprising κ-casein macropeptide and the heat labile protein, the temperature of the second proteinaceous material being the elevated temperature, and the elevated temperature effective to polymerize at least some of the heat labile protein present in the second proteinaceous material;
cooling the third proteinaceous material to yield a fourth proteinaceous material; and
passing the fourth proteinaceous material across a filtration membrane to yield a filtration retentate and a filtration permeate, the filtration permeate comprising a majority of the κ-casein macropeptide from the first proteinaceous material or a majority of the κ-casein macropeptide from the second proteinaceous material.

2. The method of claim 1 wherein the heat labile protein comprises (1) β-lactoglobulin, (2) α-lactalbumin, or (3) β-lactoglobulin and α-lactalbumin.

3. The method of claim 2 wherein the fourth proteinaceous material comprises polymers of β-lactoglobulin and κ-lactalbumin, the filtration retentate comprising a majority of the polymers of β-lactoglobulin and α-lactalbumin from the third proteinaceous material.

4. The method of claim 1 wherein the κ-casein macropeptide comprises glycomacropeptide.

5. The method of claim 1 wherein the filtration retentate comprises polymerized β-lactoglobulin or polymerized α-lactalbumin.

6. The method of claim 1 wherein the elevated temperature is greater than about 175° F.

7. The method of claim 1 wherein the elevated temperature is at least about 180° F.

8. The method of claim 1 wherein the fourth proteinaceous material has a temperature of less than about 140° F.

9. The method of claim 1, the method further comprising drying the filtration retentate to yield a polymerized protein powder.

10. The method of claim 9 wherein the polymerized protein powder is soluble in an aqueous solution, where about 100 grams of the aqueous solution includes about 10 grams of the polymerized protein powder and about 90 grams of water, the water having a temperature of about 72° F.

11. The method of claim 10 wherein none of the polymerized protein powder precipitates in the aqueous solution within a period of about one hour after preparation of the aqueous solution.

12. The method of claim 9 wherein an aqueous solution containing about 10 grams of the polymerized protein powder and about 90 grams of water has a Brookfield viscosity of at least about 6,000 centipoise when the temperature of the aqueous solution is about 72° F.

13. The method of claim 12 wherein the aqueous solution has a Brookfield viscosity of at least about 10,000 centipoise when the temperature of the aqueous solution is about 72° F.

14. The method of claim 1 wherein the filtration membrane is a microfiltration membrane

15. The method of claim 1 wherein the filtration membrane has a nominal pore diameter in the range of about 0.02 microns to less than about 0.2 microns.

16. The method of claim 1 wherein the filtration membrane has a nominal pore diameter of about 0.05 microns.

17. The method of claim 1 wherein the filtration membrane has a MWCO greater than approximately 500,000 Daltons and is made of polyvinyl difluoride.

18. The method of claim 1, the method further comprising ultrafiltering the filtration permeate to yield an ultrafiltration retentate and an ultrafiltration permeate, the ultrafiltration retentate comprising a majority of the κ-casein macropeptide from the filtration permeate.

19. The method of claim 18, the method further comprising adjusting the pH of the filtration permeate to a pH ranging from about 5.5 to about 6.5 prior to ultrafiltering the filtration permeate.

20. The method of claim 18, the method further comprising drying the ultrafiltration retentate to yield a κ-casein macropeptide-enriched powder.

21. The method of claim 20 wherein the κ-casein macropeptide-enriched powder is glycomacropeptide-enriched powder.

22. The method of claim 1, the method further comprising drying a derivative of the filtration permeate to yield a κ-casein macropeptide-enriched powder, the concentration of κ-casein macropeptide in the κ-casein macropeptide-enriched powder, based on the total protein weight of the κ-casein macropeptide-enriched powder, being at least about two times greater than the concentration of κ-casein macropeptide in the first proteinaceous material, based on the total protein weight of the first proteinaceous material, or at least about two times greater than the concentration of κ-casein macropeptide in the second proteinaceous material, based on the total protein weight of the second proteinaceous material.

23. The method of claim 1 wherein the first proteinaceous material or the second proteinaceous material comprises glycomacropeptide, the method further comprising drying a derivative of the filtration permeate to yield a glycomacropeptide-enriched powder, the concentration of glycomacropeptide in the glycomacropeptide-enriched powder, based on the total protein weight of the glycomacropeptide-enriched powder, being at least about two times greater than the concentration of glycomacropeptide in the first proteinaceous material, based on the total protein weight of the first proteinaceous material, or at least about two times greater than the concentration of glycomacropeptide in the second proteinaceous material, based on the total protein weight of the second proteinaceous material.

24. The method of claim 1, the method further comprising drying a derivative of the filtration permeate to yield a κ-casein macropeptide-enriched powder, the κ-casein macropeptide-enriched powder comprising at least about 35 weight percent κ-casein macropeptide, based on the total protein weight of the κ-casein macropeptide-enriched powder.

25. The method of claim 1 wherein the first proteinaceous material or the second proteinaceous material comprises glycomacropeptide, the method further comprising drying a derivative of the filtration permeate to yield a glycomacropeptide-enriched powder, the glycomacropeptide-enriched powder comprising at least about 35 weight percent glycomacropeptide, based on the total protein weight of the glycomacropeptide-enriched powder.

26. A method of processing a proteinaceous material, the proteinaceous material comprising κ-casein macropeptide, the method comprising:
polymerizing protein present in the proteinaceous material to yield a proteinaceous intermediate, the proteinaceous intermediate comprising polymerized protein; and
separating the proteinaceous intermediate to yield a first portion and a second portion, the first portion comprising a majority of the κ-casein macropeptide from the proteinaceous material and the second portion comprising a majority of the polymerized protein from the proteinaceous intermediate.

27. The method of claim 26, the method further comprising processing the second portion to yield a polymerized protein powder.

28. The method of claim 27 wherein the polymerized protein powder is soluble in an aqueous solution, where about 100 grams of the aqueous solution includes about 10 grams of the polymerized protein powder and about 90 grams of water, the water having a temperature of about 72° F.

29. The method of claim 27 wherein none of the polymerized protein powder precipitates in the aqueous solution within a period of about one hour after preparation of the aqueous solution.

30. The method of claim 27 wherein an aqueous solution containing about 10 grams of the polymerized protein powder and about 90 grams of water has a Brookfield viscosity of at least about 6,000 centipoise when the temperature of the aqueous solution is about 72° F.

31. The method of claim 30 wherein the aqueous solution has a Brookfield viscosity of at least about 10,000 centipoise when the temperature of the aqueous solution is about 72° F.

32. The method of claim 26, the method further comprising filtering the first portion to yield a filtration retentate and a filtration permeate, the filtration retentate comprising a majority of the κ-casein macropeptide from the proteinaceous material.

33. The method of claim 32, the method further comprising drying the filtration retentate to yield a κ-casein macropeptide-enriched powder.

34. The method of claim 33 wherein the concentration of κ-casein macropeptide in the κ-casein macropeptide-enriched powder, based on the total protein weight of the κ-casein macropeptide-enriched powder, is at least about two times greater than the concentration of κ-casein macropeptide in the proteinaceous material, based on the total protein weight of the proteinaceous material.

35. The method of claim 33 wherein the κ-casein macropeptide-enriched powder comprises at least about 35 weight percent κ-casein macropeptide, based on the total protein weight of the κ-casein macropeptide-enriched powder.

36. The method of claim 26 wherein the proteinaceous material comprises glycomacropeptide, the method further comprising filtering the first portion to yield a filtration retentate and a filtration permeate, the filtration retentate comprising a majority of the glycomacropeptide from the proteinaceous material.

37. The method of claim 36, the method further comprising drying the filtration retentate to yield a glycomacropeptide-enriched powder.

38. The method of claim 37 wherein the concentration of glycomacropeptide in the glycomacropeptide-enriched powder, based on the total protein weight Of the glycomacropeptide-enriched powder, is at least about two times greater than the concentration of glycomacropeptide in the proteinaceous material, based on the total protein weight of the proteinaceous material.

39. The method of claim 37 wherein the glycomacropeptide-enriched powder comprises at least about 35 weight percent glycomacropeptide, based on the total protein weight of the glycomacropeptide-enriched powder.

40. The method of claim 26 wherein the proteinaceous material comprises β-lactoglobulin or α-lactalbumin.

41. The method of claim 40 wherein the polymerized protein comprises polymers of β-lactoglobulin and α-lactalbumin.

42. The method of claim 26 wherein the proteinaceous material has a pH greater than about 7.

43. The method of claim 26 wherein the proteinaceous material has a temperature greater than about 175° F.

44. The method of claim 43 wherein the proteinaceous material has a pH greater than about 7.

45. The method of claim 26 wherein the proteinaceous material has a temperature of at least about 180° F.

46. A method of fractionating a proteinaceous material, the proteinaceous material comprising κ-casein macropeptide and heat labile protein, the method comprising:
providing the proteinaceous material with a combination of a pH, an elevated temperature, and a heated holding period that is effective to cause polymerization of at least some of the heat labile protein and yield a proteinaceous intermediate that comprises polymerized protein; and
separating the proteinaceous intermediate to yield a first portion and a second portion, the first portion comprising at least about 80 weight percent of the κ-casein macropeptide from the proteinaceous material and the second portion comprising at least about 80 weight percent of the polymerized protein from the proteinaceous intermediate.

47. The method of claim 46, the method further comprising:
adjusting the elevated temperature and the heated holding period to increase the quantity of κ-casein macropeptide remaining in the proteinaceous intermediate.

48. The method of claim 47 wherein the pH of the proteinaceous material is greater than about 7.

49. The method of claim 46, the method further comprising:
adjusting the elevated temperature and the heated holding period to increase the conversion of heat labile protein to polymerized protein.

50. The method of claim 46 wherein the heat labile protein comprises β-lactoglobulin or α-lactalbumin.

51. A method of fractionating a proteinaceous material, the proteinaceous material comprising κ-casein macropeptide and heat labile protein, the method comprising:
providing the proteinaceous material with a combination of a pH, an elevated temperature, and a heated holding period that is effective to cause polymerization of at least some of the heat labile protein and yield a proteinaceous intermediate that comprises polymerized protein; and
separating the proteinaceous intermediate to yield a first portion and a second portion, the first portion comprising κ-casein macropeptide from the proteinaceous material and the second portion comprising at least about 80 weight percent of the polymerized protein from the proteinaceous intermediate, the first portion and the proteinaceous material, when each subjected to the same reversed-phase high performance liquid chromatography analysis under identical analysis conditions, exhibiting the same, or essentially the same, κ-casein macropeptide profile.

52. A polymerized protein material, the polymerized protein material comprising polymerized β-lactoglobulin or polymerized α-lactalbumin, the polymerized protein material when mixed with water in a ratio of about 10 grams of the polymerized protein material to about 90 grams of water to form an aqueous solution, is soluble in the aqueous solution when the aqueous solution has a temperature of about 72° F.

53. The polymerized protein material of claim 52 wherein the polymerized protein material, when present in the aqueous solution, is effective to prevent any precipitation of the polymerized protein material from the aqueous solution within a period of about one hour after preparation of the aqueous solution.

54. The polymerized protein material of claim 52 wherein the polymerized protein material, when present in the aqueous solution, is effective to provide the aqueous solution with a Brookfield viscosity of at least about 6,000 centipoise when the temperature of the aqueous solution is about 72° F.

55. The polymerized protein material of claim 52 wherein the polymerized protein material, when present in the aqueous solution, is effective to provide the aqueous solution with a Brookfield viscosity of at least about 10,000 centipoise when the temperature of the aqueous solution is about 72° F.

56. A polymerized protein material, the polymerized protein material comprising polymers of β-lactoglobulin and α-lactalbumin, the polymerized protein material, when mixed with water in a ratio of about 10 grams of the polymerized protein material to about 90 grams of water to form an aqueous solution, effective to provide the aqueous solution with a Brookfield viscosity of at least about 6,000 centipoise when the temperature of the aqueous solution is about 72° F.

57. A κ-casein macropeptide-enriched powder, the κ-casein macropeptide-enriched powder comprising protein, the protein comprising β-lactoglobulin or α-lactalbumin and the protein further comprising κ-casein macropeptide, the κ-casein macropeptide-enriched powder having a protein concentration of at least about 75 weight percent, based upon the total weight of the κ-casein macropeptide-enriched powder, the concentration of κ-casein macropeptide in the κ-casein macropeptide-enriched powder being at least about 35 weight percent, based upon the total weight of protein in the κ-casein macropeptide-enriched powder, and the weight ratio of κ-casein macropeptide to the collective weight of β-lactoglobulin and α-lactalbumin in the κ-casein macropeptide-enriched powder being at least about 0.7.

58. The κ-casein macropeptide-enriched powder of claim 57 wherein the weight ratio of κ-casein macropeptide to the collective weight of β-lactoglobulin and α-lactalbumin in the κ-casein macropeptide-enriched powder is at least about 1.

59. The κ-casein macropeptide-enriched powder of claim 57 wherein protein of the κ-casein macropeptide-enriched powder comprises glycomacropeptide, the concentration of glycomacropeptide in the κ-casein macropeptide-enriched powder being at least about 35 weight percent, based upon the total weight of protein in the κ-casein macropeptide-enriched powder, and the weight ratio of glycomacropeptide to the collective weight of β-lactoglobulin and α-lactalbumin in the κ-casein macropeptide-enriched powder being at least about 0.7.

60. The κ-casein macropeptide-enriched powder of claim 59 wherein the weight ratio of glycomacropeptide to the collective weight of β-lactoglobulin and α-lactalbumin in the κ-casein macropeptide-enriched powder is at least about 1.
